(19) United States
(12) Patent Application Publication
(10) Pub. No.: US 2024/0321411 A1
(43) Pub. Date: Sep. 26, 2024

(54) Systems and Methods for the Direct Comparison of Molecular Derivatives

(71) Applicant: Duke University, Durham, NC (US)

(72) Inventors: Daniel REKER, Durham, NC (US); Zachary FRALISH, Durham, NC (US)

(21) Appl. No.: 18/611,203

(22) Filed: Mar. 20, 2024

Related U.S. Application Data
(60) Provisional application No. 63/453,248, filed on Mar. 20, 2023.

(51) Int. Cl. G16C 20/70; G06N 20/00; G16C 20/30

(52) U.S. Cl. CPC G16C 20/70; G06N 20/00; G16C 20/30

(57) ABSTRACT

Described herein are methods for the direct comparison of predicted properties of molecular derivatives for molecular optimization, lead series prioritization, and computational design of prodrugs that exhibit desired biological and physical properties. The described pipeline can be used to streamline the optimization of drug leads and design of prodrugs for small molecular FDA-approved drugs and investigational preclinical drug candidates.

### CROSS-REFERENCE TO RELATED APPLICATIONS

This application claims priority to U.S. Provisional Patent Application No. 63/453,248 filed on Mar. 20, 2023, which is incorporated by reference herein in its entirety.

### FEDERALLY SPONSORED RESEARCH

This invention was made with government support under grant number R35GM151255 awarded by the National Institutes of Health. The government has certain rights in the invention.

### BACKGROUND

Computational approaches are becoming increasingly employed to efficiently characterize compound properties and enable larger scale evaluations of drug candidates. Molecular machine learning algorithms learn from historic data to directly predict the absolute property values of a molecule from its chemical structure to triage experimental testing. Such machine learning workflows are becoming increasingly accurate due to expanding availability of training data, growing computational power, and improvements in predictive algorithms. However, molecular machine learning algorithms are not yet optimized to directly compare molecular properties to guide molecular derivatizations, enable lead series prioritization, and design prodrugs.

Prodrugs are drug derivatives that exhibit beneficial properties compared to their parent drugs, including improved pharmacokinetics or reduced side effects. Rational prodrug design is challenging, as it requires careful crafting of release mechanisms and holistic optimization of pharmacokinetic properties. As such, prodrugs currently make up only 10% of all approved drugs and a majority have been discovered serendipitously or rely on the attachment of simple functionalizations such as short alkanes (25%) or phosphates (15%). Increased complexity of prodrugs can enable greater pharmacokinetic control and innovative release mechanisms for enhanced tissue targeting.

Thus, there is an ongoing opportunity for improved systems and methods to develop these and other types of drug derivatives.

### SUMMARY

One embodiment described herein is a computer-implemented method for training a machine learning model for predicting molecular property differences, the method comprising: receiving a set of data including molecules, wherein each molecule of the set of data includes a molecular representation and a known absolute property value of each molecule in the set of data; creating a set of training data with the set of data; creating a set of molecule pairs using each molecule of the set of training data; generating a shared molecular representation of each pair of molecules in the set of training data; training a machine learning model of an artificial intelligence (AI) system using the set of training data, wherein the set of training data includes the shared molecular representation and property difference of each pair of molecules in the set of training data; and for two molecules forming a molecule pair, predicting a property difference of molecular derivatization using the machine learning model as trained based on property differences of each pair of molecules. In one aspect, creating the set of molecule pairs using each molecule of the set of training data, further comprises: splitting the set of training data into a training set and a test set. In another aspect, creating the set of molecule pairs using each molecule of the set of training data, further comprises: generating a pair of molecules for at least one of the training set and the test set by cross merging a first molecule of the training set or the test set and a second molecule of the training set or the test set, wherein all possible molecule pairs of the training set and the test set are generated, wherein cross merging of the training set is limited to molecules of the training set, and wherein cross merging of the test set is limited to molecules of the test set. In another aspect, generating a shared molecular representation of each pair of molecules in the set of training data, further comprises: concatenating a first molecular representation of a first molecule and a second molecular representation of a second molecule of each pair of molecules in the set of training data.

Another embodiment described herein is a computer program product comprising program instructions stored on a machine-readable storage medium, wherein when the program instructions are executed by a computer processor, the program instructions cause the computer processor to execute the method as described herein.

Another embodiment described herein is a computer-implemented method for training a machine learning model for retrieving a compound with a desired characteristic from a set of data, the method comprising: receiving a set of data including molecules, wherein each molecule of the set of data includes a molecular representation and a known absolute property; creating a set of training data based on the set of data; creating a set of molecule pairs using each molecule of the set of training data; generating a shared molecular representation of each pair of molecules in the set of molecule pairs; training a machine learning model of an AI system using the set of training data, wherein the set of training data includes the shared molecular representation and respective property differences of each pair of molecules of the set of training data; identifying a first compound of the set of training data based on a property of the identified compound; pairing the identified compound with each compound of a learning dataset, wherein the learning data set is based on the set of data; for a pair of molecules from the learning dataset, predicting a property difference of the pair of molecules from the learning dataset using the machine learning model as trained based on property differences of each pair of molecules of the set of training data, wherein the pair of molecules include the identified compound; and adding a compound paired with the identified compound from the learning dataset to the training data set based on a property increase of the compound and the identified compound. In one aspect, the method further comprises: creating a second set of molecule pairs using each molecule of the set of training data, wherein the set of training data includes the added compound; and retraining the machine learning model using the set of training data, wherein the set of training data includes shared molecular representations and respective property differences of each pair of molecules of the second set of molecule pairs of the set of training data. In another aspect, the compound paired with the identified compound has a property improvement greater than other compounds paired with the identified compound in the learning dataset. In another aspect, creating the set of molecule pairs using each molecule of the set of training data, further comprises: splitting the set of training data into one or more sets selected from the group consisting of: a training set, a test set, and the learning dataset. In another aspect, creating the set of molecule pairs using each molecule of the set of training data, further comprises: generating a pair of molecules for at least one of the training set, the test set, and the learning dataset by cross merging a first molecule and a second molecule of the training set, the test set, or the learning dataset, wherein all possible molecule pairs of the training set, the test set, or the learning dataset are generated and the learning set is cross merged with one molecule of the training set, wherein cross merging of the training set is limited to molecules of the training set, wherein cross merging of the test set is limited to molecules of the test set, and wherein cross merging of the learning set is limited to the one molecule of the training set and molecules of the learning set, wherein the one molecule of the training set includes a desired property value. In one aspect, the method further comprises: for a pair of molecules from an external dataset, predicting a property difference of the pair of molecules from the external dataset using the machine learning model as trained based on property differences of each pair of molecules of the set of training data.

Another embodiment described herein is a computer program product comprising program instructions stored on a machine-readable storage medium, wherein when the program instructions are executed by a computer processor, the program instructions cause the computer processor to execute the method as described herein.

Another embodiment described herein is a computer-implemented method for training a machine learning model for predicting which of a pair of molecules has an improved property value, the method comprising: receiving a set of data including molecules, wherein each molecule of the set of data includes a molecular representation and a value selected from a group consisting of: a known exact absolute property value and a known bound absolute property value, wherein the known exact absolute property value and the known bound absolute property value are related to a property of each molecule of the set of data; creating a set of training data with the set of data; creating a set of molecule pairs using each molecule of the set of training data; filtering the set of training data based on a set of rules, wherein the filtered set of training data includes molecule pairs of the set of molecule pairs having at least one molecule with a property value improved compared to the other molecule; training a machine learning model of an AI system using datapoints of the filtered set of training data, wherein the datapoints include molecular pairs of the filtered set of training data with shared representations, and wherein the datapoints include at least one selected from the group consisting of: bounded datapoints and exact regression datapoints; and for datapoints of a pair of molecules, predicting a property value improvement of molecular derivatization using the machine learning model as trained based on property differences of the datapoints, wherein the property value improvement indicates at least one molecule of the pair of molecules includes a property value greater than the other molecule. In one aspect, filtering the set of training data based on the set of rules, further comprises: removing, from the set of training data, molecular pairs of the set of training data with a property difference below a property difference threshold value. In another aspect, filtering the set of training data based on the set of rules, further comprises: removing, from the set of training data, molecular pairs of the set of training data having a first molecule and a second molecule with equal property values. In another aspect, filtering the set of training data based on the set of rules, further comprises: removing, from the set of training data, molecular pairs of the set of training data when an improved property is unknown. In another aspect, creating the set of molecule pairs using each molecule of the set of training data, further comprises: splitting the set of training data into a training set and a test set. In another aspect, creating the set of molecule pairs using each molecule of the set of training data, further comprises: generating a pair of molecules for at least one of the training set and the test set by cross merging a first molecule and a second molecule of the training set and the test set, wherein all possible molecule pairs of the training set and the test set are generated, wherein cross merging of the training set is limited to molecules of the training set, and wherein cross merging of the test set is limited to molecules of the test set.

Another embodiment described herein is a computer program product comprising program instructions stored on a machine-readable storage medium, wherein when the program instructions are executed by a computer processor, the program instructions cause the computer processor to execute the method as described herein.

### DESCRIPTION OF THE DRAWINGS

FIG. 1 schematically illustrates a chemical structures prioritization system according to some embodiments.

FIG. 2 schematically illustrates an example architecture tailored for paired inputs of molecules into the chemical structures prioritization system according to some embodiments.

FIG. 3 is a flowchart illustrating a method performed by the chemical structures prioritization system of FIG. 1 according to some embodiments.

FIG. 4 is a flowchart illustrating a method performed by the chemical structures prioritization system of FIG. 1 according to some embodiments.

FIG. 5 is a flowchart illustrating a method performed by the chemical structures prioritization system of FIG. 1 according to some embodiments.

FIG. 6A-C illustrate traditional and pairwise architectures according to some embodiments. FIG. 6A shows traditional molecular machine learning models take singular molecular inputs and predict absolute properties of molecules. Predicted property differences can be calculated by subtracting predicted values for two molecules. FIG. 6B shows pairwise models train on differences in properties from pairs of molecules to directly predict property changes of molecular derivatizations. FIG. 6C shows molecules are cross-merged to create pairs only after cross-validation splits to prevent the risk of data leakage during model evaluation. Therefore, every molecule in the dataset can only occur in pairs in the training or testing data, but not both.

FIG. 7 shows cross-validation results across benchmark data for various machine learning algorithms according to some embodiments. Correlation plots for Random Forest, ChemProp, DeepDelta, LGBMsub, and Delta LGBMsub following 5×10-fold cross-validation. Datasets are sorted by size from smallest (top) to largest (bottom). Coloring is based on data density with the most densely populated regions shown in yellow, medium density shown in green, and least dense regions in blue and linear interpolation between these groups.

FIG. 8 illustrate charts depicting model performance on external datasets of various machine learning algorithms according to some embodiments. Correlation plots, Pearson's r values, MAE, RMSE, and total percent of predictions correctly indicating a positive or negative change from the starting molecule for Random Forest, ChemProp, and DeepDelta models on cross-merged external test sets. Aqueous solubility is in units of log S and volume of distribution at steady state is in units of log(body/blood concentration in L/kg). Coloring is based on data density with the most densely populated regions shown in yellow, medium density shown in green, and least dense regions in blue and linear interpolation between these groups.

FIG. 9 illustrates a graph depicting a correlation plot of a DeepDelta model trained on same molecular pairs MAE with model quality according to some embodiments. Correlation plot shows the relationship between the performance of the DeepDelta model on the 10 benchmarking datasets (x axis) with the ability of the DeepDelta models trained on these data to correctly predict same molecular pairs to have no change in property values (y axis, Eq. 1).

FIG. 10 illustrate charts of distribution of training datapoints values according to some embodiments. Distribution of training datapoints across benchmarking datasets. Units are as follows shows Fraction Unbound in Brain, Log(fu,brain); Renal Clearance, Log(CLr); Free Solvation, Experimental Hydration Free Energy in Water; Microsomal Clearance, Log(mL/min/kg cleared); Hemolytic Toxicity, Log(HD₅₀); Hepatic Clearance, Log(mL/min/kg cleared); Caco2, Log(Papp); Aqueous Solubility, Log S; Volume of Distribution at Steady State (VDss), Log(Body/Blood Concentration in L/kg); Half-Life, Log(Half-Life in Hours).

FIG. 11 illustrates a graph depicting epoch optimization for DeepDelta and ChemProp according to some embodiments. Plots of model performance in terms of Pearson's r across epochs for DeepDelta (left) and ChemProp (right). Selected values were epochs=5 for DeepDelta and epochs=50 for ChemProp.

FIG. 12 illustrate charts depicting LGBMsub model performance on external datasets according to some embodiments. Correlation plots, Pearson's r values, MAE, RMSE, and total percent of predictions correctly indicating a positive or negative change from the starting molecule for control and delta LGBMsub models on cross-merged external test sets. Aqueous solubility is in units of log S and volume of distribution at steady state is in units of log(body/blood concentration in L/kg). Coloring is based on data density with the most densely populated regions shown in yellow, medium density shown in green, and least dense regions in blue and linear interpolation between these groups.

FIG. 13 illustrate graphs of zero difference predictions correlated with cross-validation performance according to some embodiments. Plots correlating the MAE of the zero difference predictions (Eq. 1) with the MAE of the cross-validation plots show higher rates of correlation (top) than plots of the inherent variance of the original dataset with the MAE of the cross-validation (bottom). This trend is maintained when outlier datasets with large variance greater than 1 are removed (eight datasets remaining, right).

FIG. 14 illustrates a graph depicting consistency in magnitude of predictions when swapping molecule order is inversely correlated with model quality according to some embodiments. Correlation plot shows the relationship between the performance of the DeepDelta model on the 10 benchmarking datasets (x axis) with the ability of the DeepDelta models trained on these data to correctly predict swapped molecular pairs to have inverted property values (y axis, Eq. 2).

FIG. 15 illustrates a graph depicting a correlation of error from additivity between three molecules and model quality according to some embodiments. Correlation plot shows the relationship between the performance of the DeepDelta model on the 10 benchmarking datasets (x axis) with the ability of the DeepDelta models trained on these data to correctly preserve additivity for predicted differences between three molecules (y axis, Eq. 3).

FIG. 16 illustrate charts depicting a comparison of error and property differences between paired datapoints across benchmark datasets according to some embodiments. Correlation plots for Random Forest (left), ChemProp (middle), and DeepDelta (right) following 5×10-fold cross-validation. Delta represents the difference in value between paired datapoints for the property of interest. Units are as follows shows Fraction Unbound in Brain, Log(fu,brain); Renal Clearance, Log(CLr); Free Solvation, Experimental Hydration Free Energy in Water; Microsomal Clearance, Log(mL/min/kg cleared); Hemolytic Toxicity, Log(HD50); Hepatic Clearance, Log(mL/min/kg cleared); Caco2, Log(Papp); Aqueous Solubility, Log S; Volume of Distribution at Steady State, Log(Body/Blood Concentration in L/kg); Half-Life, Log(Half-Life in Hours).

FIG. 17 illustrate charts depicting a comparison of absolute error and chemical similarity across benchmark datasets according to some embodiments. Correlation plots for Random Forest (left), ChemProp (middle), and DeepDelta (right) following 5×10-fold cross-validation. Delta represents the difference in values between paired datapoints for the property of interest, and the Tanimoto similarity is a measure of the similarity between the two paired structures, where a value of 1 indicates maximum similarity and 0 indicates no similarity. Units are as follows shows Fraction Unbound in Brain, Log(fu,brain); Renal Clearance, Log(CLr); Free Solvation, Experimental Hydration Free Energy in Water; Microsomal Clearance, Log(mL/min/kg cleared); Hemolytic Toxicity, Log(HD₅₀); Hepatic Clearance, Log(mL/min/kg cleared); Caco2, Log(Papp); Aqueous Solubility, Log S; Volume of Distribution at Steady State, Log(Body/Blood Concentration in L/kg); Half-Life, Log(Half-Life in Hours).

FIG. 18 illustrate charts depicting a comparison of property differences between paired datapoints and chemical similarity across benchmark datasets according to some embodiments. Correlation plots for Random Forest, ChemProp, and DeepDelta following 5×10-fold cross-validation. Delta represents the difference in value between paired datapoints for the property of interest. Units are as follows shows Fraction Unbound in Brain, Log(fu, brain); Renal Clearance, Log(CLr); Free Solvation, Experimental Hydration Free Energy in Water; Microsomal Clearance, Log(mL/min/kg cleared); Hemolytic Toxicity, Log(HD50); Hepatic Clearance, Log(mL/min/kg cleared); Caco2, Log(Papp); Aqueous Solubility, Log S; Volume of Distribution at Steady State, Log(Body/Blood Concentration in L/kg); Half-Life, Log(Half-Life in Hours).

FIG. 19 illustrate charts depicting a comparison of predictive capacity for matched and unmatched scaffold pairs across benchmark datasets according to some embodiments. Correlation plots for predictions by DeepDelta for all datapoints, unmatched scaffolds, and matched scaffolds following 5×10-fold cross-validation. Units are as follows shows Fraction Unbound in Brain, Log(fu,brain); Renal Clearance, Log(CLr); Free Solvation, Experimental Hydration Free Energy in Water; Microsomal Clearance, Log(mL/min/kg cleared); Hemolytic Toxicity, Log(HD₅₀); Hepatic Clearance, Log(mL/min/kg cleared); Caco2, Log(Papp); Aqueous Solubility, Log S; Volume of Distribution at Steady State, Log(Body/Blood Concentration in L/kg); Half-Life, Log(Half-Life in Hours). We note that, although the scaffold matching leads to smaller datasets compared to unmatched scaffolds, this matching was only done in the test-fold and not the training-fold. Therefore, the same model is compared in all comparisons and model performance is not impacted by the size of the datasets created through matching scaffolds.

FIG. 20A-B illustrate a comparison of active learning approaches and architectures according to some embodiments. FIG. 20A shows classic exploitative active learning uses individual molecular representations to predict absolute property values to select molecules from the learning set to add into the training set. FIG. 20B shows ActiveDelta learning uses paired molecular representations to predict molecular property improvements from the currently best training compound to select the best molecule to add to the training set.

FIG. 21A-D illustrate charts depicting a comparison of performances of exploitative active learning approaches according to some embodiments. FIG. 21A-C show the percentage of the top ten percentile most potent molecules (‘hits’) in the learning set identified over 100 iterations of active learning by AD-CP (ADCP) and Chemprop (CP) (FIG. 21A), AD-XGB (ADXGB) and XGBoost (XGB) (FIG. 21B), and Random Forest (RF) and random selection (Random) (FIG. 21C). Average and standard error of the mean for three replicates across 99 Ki datasets after starting with two random datapoints is presented. FIG. 21D shows a bar chart of the identified hits of each approach at 100 iterations.

FIG. 22A-F illustrate charts depicting an exploration of chemical space for active learning approaches according to some embodiments. FIG. 22A-F show T-SNE of representative dataset (CHEMBL 232-1, Alpha-1b adrenergic receptor) highlighting molecules identified in the first 45 iterations for AD-CP (FIG. 22A), Chemprop (FIG. 22B), AD-XGB (FIG. 22C), XGBoost (FIG. 22D), Random Forest (FIG. 22E), and random selection (FIG. 22F). Top ten percentile most potent compounds are shown as stars and identified compounds are highlighted in yellow. The number of times a model ‘jumps’ from one cluster to another is shown in a green bar while the times it ‘stays’ in the same cluster is shown in light blue. Arrow gradient towards darker grey indicates increasing iteration number.

FIG. 23 illustrates a graph depicting scaffold selection for active learning approaches during exploitative active learning according to some embodiments. The number of unique scaffolds selected by random selection (Random), Random Forest (RF), Chemprop (CP), AD-CP (ADCP), XGBoost (XGB), and AD-XGB (ADXGB) is plotted over 100 iterations of active learning across 99 potency datasets after starting from two random datapoints for three repeats.

FIG. 24A-B illustrate charts depicting schematic of classification approaches to handle datasets including molecules with datapoints having exact and bounded values according to some embodiments. FIG. 24A shows schematic of classification approaches to handle bounded data 230 IC₅₀ datasets were analyzed from ChEMBL33 that included molecules with exact and bounded values, making up 20% of all datapoints. FIG. 24B shows traditional regression models cannot incorporate bounded datapoints into training. FIG. 24C shows using traditional regression models, classifications of predicted improvements can be calculated by first subtracting predictions for each molecule and then subsequently determining a class value. FIG. 24D shows pairwise model approaches can train on classified improvements from pairs of molecules, allowing for incorporation of bounded datapoints. FIG. 24D shows DeltaClassifiers can directly predict molecular improvements of molecular derivatizations from paired molecular representations.

FIG. 25A-B illustrate charts depicting DeepDeltaClassifier performance following training with only exact values, All Data, and Demilitarized Data according to some embodiments. FIG. 25A shows violin plots of model performance following 1×10 cross-validation for 230 ChEMBL datasets in terms of accuracy, F1 score, and ROCAUC. FIG. 25B shows pie charts showing percentage of datasets D×C outcompeted D×COE and D×CAD.

FIG. 26A-C illustrate charts depicting comparisons of DeltaClassifiers with traditional methods according to some embodiments. FIG. 26A shows violin plots of average model performance following 3×10 cross-validation for 230 ChEMBL datasets in terms of accuracy, F1-score, and ROCAUC. FIG. 26B shows pie charts showing percentage of datasets our DeepDeltaClassifier (D×C) outcompeted (purple), exhibited no statistical difference (gradient), or underperformed compared to Random Forest (RF, red), XGBoost (XGB, black), and ChemProp (CP, blue), and DeltaClassifierLite (ΔCL, green) in terms of accuracy, F1-score, and ROCAUC. Statistical significance from paired t-test for three repeats (p<0.05). FIG. 26C shows Z-scores for model performance in terms of accuracy, F1 score, and ROCAUC.

FIG. 27 illustrates a Cross-Validation Scheme for DeltaClassifiers according to some embodiments. Datapoints undergo cross-merging to generate pairs following cross-validation splits only to circumvent data leakage risk. As such, each molecule from the original training dataset only occurs in molecule pairs within the training or testing data splits, but never both. Additionally, if it is unknown if the property is improved (e.g., both molecules' properties are denoted as ‘>’ some value) or the difference is less than 0.1 pIC₅₀, the pair is removed.

FIG. 28A-B illustrate charts depicting Tree-based DeltaClassifier Performance Following Training with Only Exact Values, All Data, and Demilitarized Data according to some embodiments. FIG. 28A shows violin plots of model performance following 1×10 cross-validation for 230 ChEMBL datasets in terms of accuracy, F1 score, and AUC. FIG. 28B shows pie charts showing percentage of datasets ΔCL outcompeted ΔCLOE and ΔCLAD.

FIG. 29 illustrate charts depicting a Tree-based DeltaClassifier Performance compared with Traditional Models according to some embodiments. Pie charts show the percentage of datasets the tree-based DeltaClassifier (ΔCL) outcompeted (green), exhibited a non-significant difference (gradient), or underperformed Random Forest (RF, red), XGBoost (XGB, black), and ChemProp (CP, blue) during 3×10-fold cross-validation. Statistical significance from paired t-test for three repeats (p<0.05).

FIG. 30A-B illustrate charts depicting Modified Z-Score Calculations according to some embodiments. FIG. 30A shows average modified Z-scores for model (Random Forest (RF), XGBoost (XGB), ChemProp (CP), tree-based DeltaClassifier (ΔCL), and DeltaClassifier (D×C)) performance following 3×10 cross-validation for 230 ChEMBL datasets in terms of accuracy, F1 score, and AUC. FIG. 30B shows median and 95% confidence interval of average modified z-scores.

FIG. 31 illustrate graphs of Percent of Bounded Data Correlated with Improvement Over Traditional Models according to some embodiments. Scatterplots showing correlation and

Pearson's r values of tree-based DeltaClassifier (ΔCL) performance improvement over Random Forest (RF), XGBoost (XGB) ChemProp (CP), and tree-based DeltaClassifier trained only on exact values (ΔCLOE) following 1×10 cross-validation for 230 ChEMBL datasets with the percent of bounded data within each dataset in terms of accuracy, F1 score, and AUC.

FIG. 32 illustrate graphs of Limited Correlation of Dataset Size with Model Performance according to some embodiments. Scatterplots showing correlation and Pearson's r values of model performance following 1×10 cross-validation for 230 ChEMBL datasets with dataset size in terms of accuracy, F1 score, and AUC with dataset size for Random Forest (RF), XGBoost (XGB), ChemProp (CP), tree-based DeltaClassifier trained on only exact data (ΔCLOE), tree-based DeltaClassifier (ΔCL), deep DeltaClassifier trained on only exact data (D×COE), and deep DeltaClassifier (D×C).

FIG. 33A-L illustrate graphs depicting Comparisons of DeltaClassifiers with Traditional Methods Across Scaffold Splits according to some embodiments. FIG. 33A shows violin plots of model performance following 1×10 cross-validation for non-matching scaffold pairs for 230 ChEMBL datasets in terms of accuracy, F1-score, and ROCAUC. FIG. 33B shows pie charts showing percentage of datasets our DeepDeltaClassifier (D×C) outcompeted Random Forest (RF), XGBoost (XGB), ChemProp (CP), and DeltaClassifierLite (ΔCL) in terms of accuracy, F1-score, and ROCAUC for non-matching scaffold pairs. FIG. 33C shows Z-scores for model performance in terms of accuracy, F1 score, and ROCAUC for non-matching scaffolds. FIG. 33D shows median and 95% confidence interval of z-scores for non-matching scaffold pairs. FIG. 33E shows modified Z-scores for model performance for non-matching scaffold pairs following 1×10 cross-validation for 230 ChEMBL datasets in terms of accuracy, F1 score, and AUC. FIG. 33F shows median and 95% confidence interval of modified z-scores for non-matching scaffold pairs. FIG. 33G shows violin plots of model performance following 1×10 cross-validation for matching scaffold pairs for 230 ChEMBL datasets in terms of accuracy, F1-score, and ROCAUC. FIG. 33H shows pie charts showing percentage of datasets our D×C outcompeted RF, XGB, CP, and ΔCL in terms of accuracy, F1-score, and ROCAUC for matching scaffold pairs. FIG. 33I shows Z-scores for model performance in terms of accuracy, F1 score, and ROCAUC for matching scaffold pairs. FIG. 33J shows median and 95% confidence interval of z-scores for matching scaffold pairs. FIG. 33K shows modified Z-scores for model performance for matching scaffold pairs following 1×10 cross-validation for 230 ChEMBL datasets in terms of accuracy, F1 score, and AUC. FIG. 33L shows median and 95% confidence interval of modified z-scores for matching scaffold pairs.

### DETAILED DESCRIPTION

Unless otherwise defined, all technical and scientific terms used herein have the same meaning as commonly understood by one of ordinary skill in the art. For example, any nomenclatures used in connection with, and techniques of computer science, pharmaceuticals, biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not. As used herein, “comprising,” is an “open-ended” term that does not exclude additional, unrecited elements or method steps. As used herein, “consisting essentially of” limits the scope of a claim to the specified materials or steps and those that do not materially affect the basic and novel characteristics of the claimed invention. As used herein, “consisting of” excludes any element, step, or ingredient not specified in the claim.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjuctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points, or as described above in the definition of “about.”

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e., living organism, such as a patient). In some embodiments, the subject comprises a human who is undergoing a treatment using a system or method as prescribed herein.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

One embodiment described herein is a method for the computational comparison of two molecules to guide molecular optimization. The described pipeline can be used to economize and accelerate compound characterization while enabling the evaluation of larger sets of candidates by informing lead series prioritization through direct contrasting of expected molecular properties.

Another embodiment described herein is a method for the computational design of prodrugs to exhibit the desired biological and physical properties. The described pipeline can be used to streamline the design of prodrugs for small molecular FDA-approved drugs and investigational preclinical drug candidates. These methods, described in further detail below, can enable the design of next-generation prodrugs with desired properties. Compared to other advanced drug delivery strategies, prodrugs developed using the disclosed method can be easier to synthesize, more readily orally bioavailable, and more stable, thereby increasing their translatability into low resource settings and improving global health and medication equity. Additionally, the methods described herein will also expand the predictive toolbox for drug design, medicinal chemistry, and drug-drug interactions.

### Methods for the Design of Molecular Derivatives

### Machine Learning Models to Predict Property Improvements of Molecular Derivatives by Comparing Two Molecules

Typically, molecular machine learning models receive only one molecule as input and predict their absolute biological and physical properties. These global models lack molecular resolution to predict property differences between similar molecular structures, which are important to predict for molecular optimization tasks. The pairwise data selection based on molecules with shared scaffolds leads to a combinatorial expansion of data. Different machine learning models that scale well with large datasets were implemented (e.g., tree-based models including Random Forest and XGBoost, deep neural networks based on graph-convolution and transformer networks). Models will be evaluated retrospectively using cross-validations and external validation sets. Other embodiments optimize global models to predict absolute property values and quantify predicted differences while considering predictive uncertainty. Other embodiments can utilize integrated in vitro testing to improve the resolution of molecular machine learning for specific molecular derivatives.

Another aspect of the present disclosure provides a computing system configured to carry out the foregoing methods. The system can comprise any suitable components, which will be evident to a person of skill in the art. The components can include, but are not limited to, a processor, a memory, a computing platform, and a software algorithm.

The systems and methods described herein can be implemented in hardware, software, firmware, or combinations of hardware, software and/or firmware. In some examples, the systems and methods described in this specification may be implemented using a non-transitory computer readable medium storing computer executable instructions that when executed by one or more processors of a computer cause the computer to perform operations. Computer readable media suitable for implementing the systems and methods described in this specification include non-transitory computer-readable media, such as disk memory devices, chip memory devices, programmable logic devices, random access memory (RAM), read only memory (ROM), optical read/write memory, cache memory, magnetic read/write memory, flash memory, and application-specific integrated circuits. In addition, a computer readable medium that implements a system or method described in this specification may be located on a single device or computing platform or may be distributed across multiple devices or computing platforms.

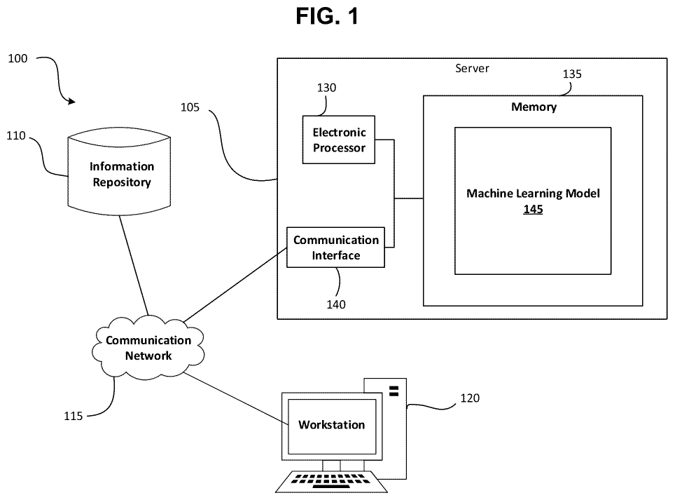

Embodiments described provided herein provide methods and systems for prioritizing chemical structures by accurately anticipating biological and physical properties of novel molecules. FIG. 1 illustrates a chemical structures prioritization system 100 according to some embodiments. As illustrated in FIG. 1, the system 100 includes a server 105, an information repository 110, and a workstation 120. The server 105, the information repository 110, and the workstation 120 communicate over one or more wired or wireless communication networks 115. Portions of the wireless communication networks 115 may be implemented using a wide area network, such as the Internet, a local area network, such as a Bluetooth™ network or Wi-Fi, and combinations or derivatives thereof. It should be understood that the system 100 may include more or fewer servers and the single server 105 illustrated in FIG. 1 is purely for illustrative purposes. For example, in some embodiments, the functionality described herein is performed via a plurality of servers in a distributed or cloud-computing environment. Also, in some embodiments, the server 105 may communicate with multiple information repositories. Additionally, it should be understood that the system 100 may include more workstations and the single workstation 120 illustrated in FIG. 1 is purely for illustrative purposes. For example, in some embodiments, the system 100 includes a plurality of workstations 120, each workstation associated with a care provider. Also, in some embodiments, the components illustrated in system 100 may communicate through one or more intermediary devices (not shown).

The information repository 110 stores datasets, including, for example, molecule data. The molecule data may comprise a molecular representation, a known absolute property value, and/or a known absolute potency of a molecule. The molecule data may also comprise a bounded and/or exact regression datapoint related to the molecule. For example, a bounded datapoint of a molecule includes a known exact absolute property value a known bound absolute property value of the molecule. In some embodiments, the information repository 110 may also be included as part of the server 105. Also, in some embodiments, the information repository 110 may represent multiple servers or systems. Accordingly, the server 105 may be configured to communicate with multiple systems or servers to perform the functionality described herein. Alternatively, or in addition, the information repository 110 may represent an intermediary device configured to communicate with the server 105 and one or more additional systems or servers.

As illustrated in FIG. 1, the server 105 includes an electronic processor 130, a memory 135, and a communication interface 140. The electronic processor 130, the memory 135, and the communication interface 140 communicate wirelessly, over wired communication channels or buses, or a combination thereof. The server 105 may include additional components than those illustrated in FIG. 1 in various configurations. For example, in some embodiments, the server 105 includes multiple electronic processors, multiple memory modules, multiple communication interfaces, or a combination thereof. Also, it should be understood that the functionality described herein as being performed by the server 105 may be performed in a distributed nature by a plurality of computers located in various geographic locations. For example, the functionality described herein as being performed by the server 105 may be performed by a plurality of computers included in a cloud computing environment.

The electronic processor 130 may be, for example, a microprocessor, an application-specific integrated circuit (ASIC), and the like. The electronic processor 130 is generally configured to execute software instructions to perform a set of functions, including the functions described herein. The memory 135 includes a non-transitory computer-readable medium and stores data, including instructions executable by the electronic processor 130. The communication interface 140 may be, for example, a wired or wireless transceiver or port, for communication over the communication network 115 and, optionally, one or more additional communication networks or connections.

As illustrated in FIG. 1, the memory 135 of the server 105 includes a machine learning model 145, which may be part of a chemical structure prioritization engine executed via the server 105. The model 145 may be, for example, an artificial intelligence system. As a pair of molecules is received, the server 105 uses the model 145 to determine a characteristic of a molecular derivatization of the pair of molecules. The characteristic may include, for example, a property difference, a potency difference, and/or a potency improvement of the molecular derivatization. In some instances, the potency improvement relates to a value indicating an increase in potency.

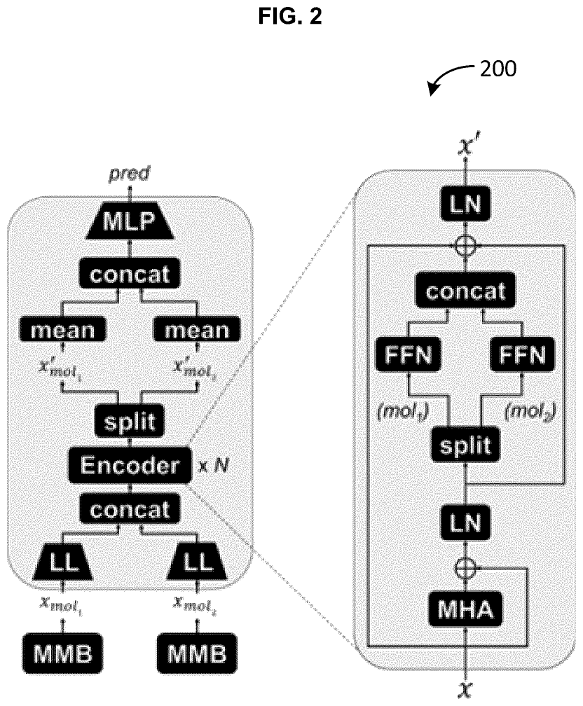

FIG. 2 illustrates an example architecture tailored for paired inputs of molecules into the chemical structures prioritization system 100 of FIG. 1 according to some embodiments. As illustrated in FIG. 2, two molecules (mol1 and mol2) are represented using standard molecular representations, such as the MegaMolBart (MMB) representation (NVIDIA), in a dimension such as, 512 by the length of their canonical SMILES representation. They are further processed by Linear layers (LL) further process the molecules to reduce the dimension (for example, to 256). The representations output from the linear layers are then concatenated (concat) together along the SMILES axis. Within the encoder, multiheaded attention (MHA) is used to jointly attend to information from different subspaces, splitting the concatenated representation (for example, into 4 heads each with a dimension of 64). Skip connections are used to preserve information that may be lost or diluted by passing through multiple layers. Layer normalization (LN) is applied to normalize across all features for each of the inputs to a specific layer to prevent gradient explosion and reduce training time. The molecular representations are then split and passed individually through feed-forward networks (FFN) to parameterize the ‘re-averaging’ of self-attention by passing through linear transformations with applied activation functions (for example, Gaussian Error Gated Linear Units (GEGLU)). Subsequent drop out and linear layers to return the dimensionality (for example, 256). The molecules are split again and averaged along tokens to get a single molecule representation instead of single character (mean) and then concatenated (concat) along the embedding dimensions back to original dimension size. A final multilayer perceptron (MLP) reduces the dimension down to a singular value by projecting down the prediction heads. Loss functions (for example, optimization on mean squared error) guide training while learning rate schedulers (for example, modified cosine annealing coupled to an AdamW optimizer) guide that rate at which the model parameters adjusted across epochs.

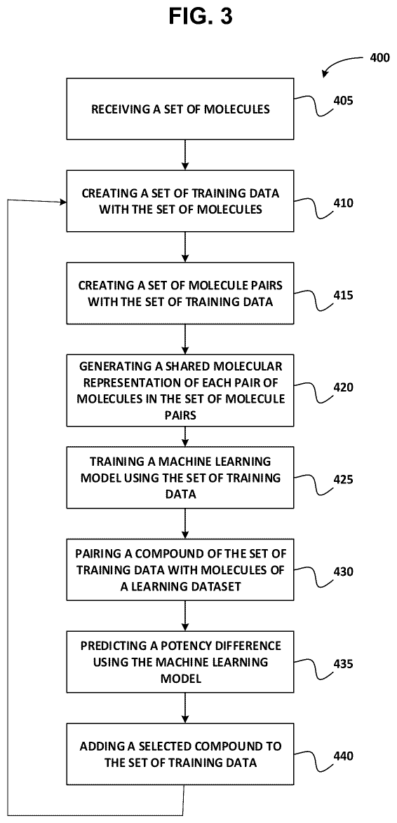

In some embodiments, the model 145 implements a DeepDelta approach. DeepDelta is a novel pairwise learning approach that simultaneously processes molecules in pairs and learns to predict property differences between the two molecules. DeepDelta provides an advantage over conventional by transforming a classically single molecule task into a dual molecule task by pairing molecules. This transformation creates a new regression task with quadratically increased training data amounts. The new task allows machine learning models to directly predict molecular property differences, which is directly poised to support medicinal chemistry derivatization pursuits including molecular optimization, lead series prioritization, and prodrug design. For example, FIG. 3 is a flowchart illustrating a method 300 for method for training a machine learning model for predicting molecular property differences of molecule pairs. Various properties of molecules are used herein including potency, absorption, distribution, metabolism, excretion, and toxicity. The method 300 may be performed by the server 105 (i.e., the electronic processor 130 implementing the model 145). However, in other embodiments, the method 300 may be performed by multiple servers or systems in various configurations and distributions. The method 300 includes receiving a set of molecule data and associated information as described above (at block 305). For example, a set of data including molecules may be uploaded to the information repository 110, and the server 105 may receive the set of data including molecules and use set of data including molecules to access or receive associated information regarding a molecular representation and a known absolute property value of each molecule in the set of data from (e.g., through a push or pull configuration) the information repository 110, other data sources, or a combination thereof as described above.

The method 300 includes creating a set of training data with the set of molecule data (at block 310). For example, the server 105 may utilize the set of data including molecules and the associated information uploaded to the information repository 110 to create a set of training data. In this example, the server 105 may split the set of training data into a training set and a test.

The method 300 includes creating a set of molecule pairs with the set of training data (at block 315). For example, the server 105 creates, by cross-merging, a set of molecule pairs in the training set and the test set using each molecule of the respective training set and the respective test set. The cross-merging results in an expanded amount of molecule data in each of the training set and the test set.

The method 300 includes generating a shared molecular representation of each pair of molecules in the set of training data (at block 320). For example, the server 105 may append/concatenate together molecular representation of a first molecule and a second molecule of a molecule pair. In some implementations, the server 105 generates a shared molecular representation of a pair of molecules by concatenating, with the AI system, a first molecular representation of a first molecule and a second molecular representation of a second molecule of the pair of molecules.

The method 300 includes training a machine learning model using the set of training data (at block 325). For example, the server 105 uses shared molecular representations and respective property differences of each pair of molecules in the set of training data to train the model 145. In this example, the server 105 uses the respective property differences as the objective variable instead of the absolute property value of a single molecule. The property differences enable the model 145 to directly learn property differences from molecular pairs instead of learning absolute property values from single molecules. In other examples, the set of training data may include information, such as input-output pairs, in memory 135. The input-output pairs may include a set of features of a shared molecular representation (e.g., input) and a property difference (e.g., output) corresponding to the set features. As noted above, the labels may be defined manually by an expert or determined based on another methodology.

The method 300 includes predicting a property difference using the machine learning model trained on the set of training data (at block 330). For example, the server 105 inputs two molecules forming a molecule pair into the model 145. In this example the server 105 receives from the model 145, a predicted property difference of molecular derivatization the molecule pair, which eliminates the need of subsequent subtraction of predictions to approximate differences between molecules required in conventional system.

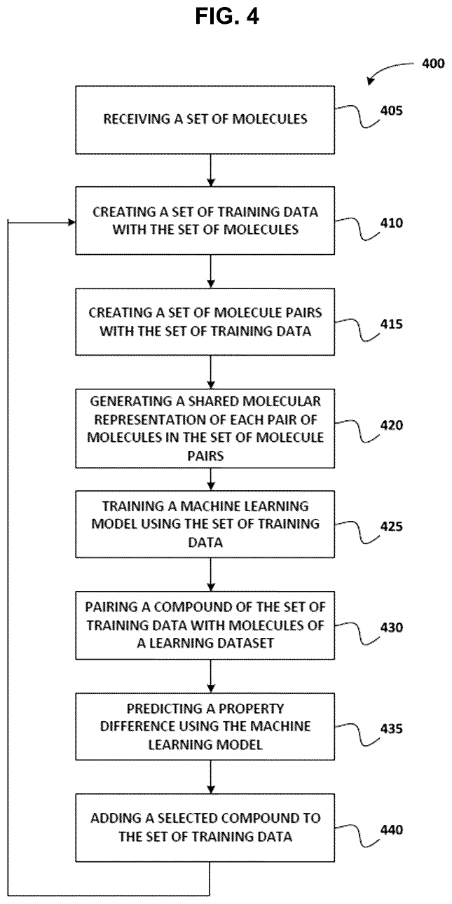

In some embodiments, the model 145 implements an ActiveDelta approach. ActiveDelta is the application of the DeepDelta approach to exploitative active learning. In conventional systems, during exploitative active learning, the next compound to be added to the training dataset is selected based on a compound from a learning set has the highest predicted property. Various properties of molecules are used herein including potency, absorption, distribution, metabolism, excretion, and toxicity. For ActiveDelta, the next compound selected is instead based on which compound of the learning set has the greatest predicted improvement from the compound, with a desired characteristic/property currently, in the training set. ActiveDelta has particular applicability for adaptive machine learning in low data regimes to guide lead optimization and prioritization during drug development. For example, FIG. 4 is a flowchart illustrating a method 400 for method for training a machine learning model for retrieving a compound from a set based on desired characteristic. The method 400 may be performed by the server 105 (i.e., the electronic processor 130 implementing the model 145). However, in other embodiments, the method 400 may be performed by multiple servers or systems in various configurations and distributions. The method 400 includes receiving a set of molecule data and associated information as described above (at block 405). For example, a set of data including molecules may be uploaded to the information repository 110, and the server 105 may receive the set of data including molecules and use the set of data including molecules to access or receive associated information regarding a molecular representation and a known absolute property value of each molecule in the set of data from (e.g., through a push or pull configuration) the information repository 110, other data sources, or a combination thereof as described above.

The method 400 includes creating a set of training data with the set of molecule data (at block 410). For example, the server 105 may utilize the set of data including molecules and the associated information uploaded to the information repository 110 to create a set of training data. In this example, the server 105 may split the set of training data into a training set, a test set, and a learning dataset. In some instances, the server 105 generates the learning data set by splitting the training set into two separate datasets.

The method 400 includes creating a set of molecule pairs with the set of training data (at block 415). For example, the server 105 creates, by cross-merging, a set of molecule pairs in the training set, the test set, and the learning dataset using each molecule of the respective training set, the respective test set, and the respective learning dataset. The cross-merging results in an expanded amount of molecule data in each of the training set, the test set, and the learning dataset. In some embodiments, the learning set is cross merged with one molecule of the training set, and the one molecule includes a desired property value (e.g., a molecule with the highest property value compared to other molecules in the training set).

The method 400 includes generating a shared molecular representation of each pair of molecules in the set of training data (at block 420). For example, the server 105 may append/concatenate together molecular representation of a first molecule and a second molecule of a molecule pair. In some implementations, the server 105 generates a shared molecular representation of a pair of molecules by concatenating, with the AI system, a first molecular representation of a first molecule and a second molecular representation of a second molecule of the pair of molecules.

The method 400 includes training a machine learning model using the set of training data (at block 425). For example, the server 105 uses shared molecular representations and respective property differences of each pair of molecules in the set of training data to train the model 145. In this example, the server 105 uses the respective property differences as the objective variable instead of the absolute property value of a single molecule. The property differences enable the model 145 to directly learn property differences from molecular pairs instead of learning absolute property values from single molecules.

The method 400 includes paring a compound of the set of training data with molecules of the learning dataset (at block 430). For example, the server 105 identifies a first compound of the set of training data based on a ground truth property of the identified compound. In some instances, the server 105 compares ground truth property of each compound of the set of training data and selects the compound with the highest property value (e.g., the highest increase in property over a molecule). In other instances, the server 105 ranks ground truth property of each compound of the set of training data and selects the compound with the highest property value. In this example, the server 105 pairs the identified compound with all compounds in the leaning dataset.

The method 400 includes predicting a property difference using the machine learning model trained on the set of training data (at block 435). For example, the server 105 inputs two molecules forming a molecule pair from the learning data set into the model 145. The molecule pair includes the identified compound. In this example, the server 105 receives from the model 145, a predicted property difference of molecular derivatization for the molecule pair.

The method 400 includes adding a compound paired with the identified compound from the learning dataset to the training data set based on a property increase of the compound and the identified compound (at block 440). For example, the server 105 selects the molecule in the learning dataset with the highest increase in the property over the identified compound. In contrast, conventional approaches simply predict the absolute property of each molecule in the learning dataset and select the molecule with the highest predicted absolute property value. In this example, the server 105 adds the selected molecule in the learning dataset with the highest increase in the property over the identified compound to the set of training data.

In some embodiments, the server 105 repeats blocks 410-440 for a defined number of iterations. For example, the server 105 creates a second set of molecule pairs using each molecule of the set of training data, wherein the set of training data includes the selected molecule. The server 105 may retrain the model 145 using the set of training data, wherein the set of training data includes shared molecular representations and respective property differences of each pair of molecules of the second set of molecule pairs of the set of training data.

In some embodiments, the server 105 receives a pair of molecules from an external dataset of the information repository 110. The server 105 inputs the pair of molecules into a trained instance of the model 145. The server 105 receives from the model 145, a predicted property difference of molecular derivatization for the pair of molecules from the external dataset of the information repository 110. In some instances, the pair of molecules includes a combination of molecules not found in the training set, the test set, or the learning dataset.

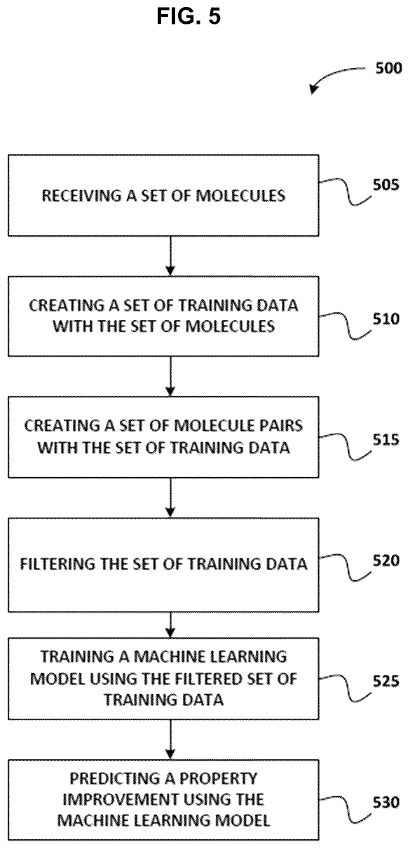

In some embodiments, the model 145 implements a DeltaClassifier approach. DeltaClassifier is a transformation of this pairing approach into a novel classification problem where the algorithm is tasked to predict which of the two paired molecules is more potent. This enables machine learning models to assess bounded datapoints by pairing them with other molecules that are known to be either more or less potent. Providing this data to a classification algorithm can create a predictive tool that directly contrasts molecules to guide molecular optimization while incorporating all the available training data (notably bounded datapoints). DeltaClassifier has particular applicability for applying smaller datasets with bounded or noisy data to train machine learning models to guide molecular optimization. For example, FIG. 5 is a flowchart illustrating a method 500 for training a machine learning model for predicting which of a pair of molecules has a greater property value. Various properties of molecules are used herein including potency, absorption, distribution, metabolism, excretion, and toxicity. The method 500 may be performed by the server 105 (i.e., the electronic processor 130 implementing the model 145). However, in other embodiments, the method 500 may be performed by multiple servers or systems in various configurations and distributions. The method 500 includes receiving a set of molecule data and associated information as described above (at block 305). For example, a set of data including molecules may be uploaded to the information repository 110, and the server 105 may receive the set of data including molecules and use set of data including molecules to access or receive associated information regarding a molecular representation and a known exact absolute property value or a known bound absolute property value in the set of data from (e.g., through a push or pull configuration) the information repository 110, other data sources, or a combination thereof as described above. The known exact absolute property value or the known bound absolute property value may relate to potency, absorption, distribution, metabolism, excretion, and toxicity of the molecular representation.

The method 500 includes creating a set of training data with the set of molecule data (at block 510). For example, the server 105 may utilize the set of data including molecules and the associated information uploaded to the information repository 110 to create a set of training data. In this example, the server 105 may split the set of training data into a training set, a test set, and a validation set.

The method 500 includes creating a set of molecule pairs with the set of training data (at block 515). For example, the server 105 creates, by cross-merging, a set of molecule pairs in the training set, the test set, and the validation set using each molecule of the respective training set, the respective test set, and the respective validation set. The cross-merging results in an expanded amount of molecule data in each of the training set, the test set, and the validation set. However, after cross-merging only the molecule pairs where it is known which molecule is more potent is kept in the set of training data. In some embodiments, the server 105 generates a shared molecular representation of each pair of molecules in the set of training data. For example, the server 105 may append together molecular representation of a first molecule and a second molecule of a molecule pair. In some implementations, the server 105 generates a shared molecular representation of a pair of molecules by concatenating, with the AI system, a first molecular representation of a first molecule and a second molecular representation of a second molecule of the pair of molecules.

The method 500 includes filtering the set of training data based on a set of rules (at block 520). For example, the server 105 uses a tunable parameter of ‘demilitarization’ to remove molecular pairs of the set of training data. In some instances, the server 105 removes molecular pairs from the set of training data when the molecular pairs have a property difference below a property difference threshold value. In other instances, the server 105 also removes molecular pairs from the set of training data when a first molecule and a second molecule of the molecular pairs have equal potencies. In some embodiments, the server 105 also removes molecular pairs of the set of training data when an improved property of the molecular pair is unknown.

The method 500 includes training a machine learning model using the filtered set of training data (at block 525). For example, the server 105 uses datapoints including shared molecular representations of molecular pairs to train the model 145. The datapoints may include bounded datapoints and exact regression datapoints. In this example, the server 105 uses the classification of property improvements between molecular pairs as the objective variable instead of the absolute property value of a single molecule. The improvements between molecular pairs enable the model 145 to directly learn property improvements from molecular pairs and instead of learning absolute property values from single molecules. In addition, the improvements between molecular pairs enable the model 145 to directly classify molecular property improvements from regression data instead of requiring subsequent subtraction of predictions to classify property improvements. In other examples, the set of training data may include information, such as input-output pairs, in memory 135.

The method 500 includes predicting a property improvement using the machine learning model trained on the set of training data (at block 330). For example, the server 105 inputs two datapoints forming a molecule pair into the model 145. In this example the server 105 receives from the model 145, a predicted property improvement of molecular derivatization of the molecule pair, which eliminates the need of subsequent subtraction of predictions to classify property improvements in conventional system.

Many different arrangements of the various components depicted, as well as components not shown, are possible without departing from the spirit and scope of the present disclosure. Embodiments of the present disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the present disclosure. It should thus be noted that the matter contained in the above description or shown in the accompanying drawings is to be interpreted as illustrative and not in a limiting sense.

It should also be noted that a plurality of hardware and software-based devices, as well as a plurality of different structural components may be utilized in various implementations. Aspects, features, and instances may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one instance, the electronic based aspects of the invention may be implemented in software (for example, stored on non-transitory computer-readable medium) executable by one or more processors. As a consequence, it should be noted that a plurality of hardware and software-based devices, as well as a plurality of different structural components may be utilized to implement the invention. For example, “control units” and “controllers” described in the specification can include one or more electronic processors, one or more memories including a non-transitory computer-readable medium, one or more input/output interfaces, and various connections (for example, a system bus) connecting the components.

It should also be understood that although certain drawings illustrate hardware and software located within particular devices, these depictions are for illustrative purposes only. In some embodiments, the illustrated components may be combined or divided into separate software, firmware, and/or hardware. For example, instead of being located within and performed by a single electronic processor, logic and processing may be distributed among multiple electronic processors. Regardless of how they are combined or divided, hardware and software components may be located on the same computing device or may be distributed among different computing devices connected by one or more networks or other suitable connections or links. Thus, in the claims, if an apparatus or system is claimed, for example, as including an electronic processor or other element configured in a certain manner, for example, to make multiple determinations, the claim or claim element should be interpreted as meaning one or more electronic processors (or other element) where any one of the one or more electronic processors (or other element) is configured as claimed, for example, to make some or all of the multiple determinations collectively. To reiterate, those electronic processors and processing may be distributed.

One embodiment described herein is a computer-implemented method for training a machine learning model for predicting molecular property differences, the method comprising: receiving a set of data including molecules, wherein each molecule of the set of data includes a molecular representation and a known absolute property value of each molecule in the set of data; creating a set of training data with the set of data; creating a set of molecule pairs using each molecule of the set of training data; generating a shared molecular representation of each pair of molecules in the set of training data; training a machine learning model of an artificial intelligence (AI) system using the set of training data, wherein the set of training data includes the shared molecular representation and property difference of each pair of molecules in the set of training data; and for two molecules forming a molecule pair, predicting a property difference of molecular derivatization using the machine learning model as trained based on property differences of each pair of molecules. In one aspect, creating the set of molecule pairs using each molecule of the set of training data, further comprises: splitting the set of training data into a training set and a test set. In another aspect, creating the set of molecule pairs using each molecule of the set of training data, further comprises: generating a pair of molecules for at least one of the training set and the test set by cross merging a first molecule of the training set or the test set and a second molecule of the training set or the test set, wherein all possible molecule pairs of the training set and the test set are generated, wherein cross merging of the training set is limited to molecules of the training set, and wherein cross merging of the test set is limited to molecules of the test set. In another aspect, generating a shared molecular representation of each pair of molecules in the set of training data, further comprises: concatenating a first molecular representation of a first molecule and a second molecular representation of a second molecule of each pair of molecules in the set of training data.

Another embodiment described herein is a computer program product comprising program instructions stored on a machine-readable storage medium, wherein when the program instructions are executed by a computer processor, the program instructions cause the computer processor to execute the method as described herein.

Another embodiment described herein is a computer-implemented method for training a machine learning model for retrieving a compound with a desired characteristic from a set of data, the method comprising: receiving a set of data including molecules, wherein each molecule of the set of data includes a molecular representation and a known absolute property; creating a set of training data based on the set of data; creating a set of molecule pairs using each molecule of the set of training data; generating a shared molecular representation of each pair of molecules in the set of molecule pairs; training a machine learning model of an AI system using the set of training data, wherein the set of training data includes the shared molecular representation and respective property differences of each pair of molecules of the set of training data; identifying a first compound of the set of training data based on a property of the identified compound; pairing the identified compound with each compound of a learning dataset, wherein the learning data set is based on the set of data; for a pair of molecules from the learning dataset, predicting a property difference of the pair of molecules from the learning dataset using the machine learning model as trained based on property differences of each pair of molecules of the set of training data, wherein the pair of molecules include the identified compound; and adding a compound paired with the identified compound from the learning dataset to the training data set based on a property increase of the compound and the identified compound. In one aspect, the method further comprises: creating a second set of molecule pairs using each molecule of the set of training data, wherein the set of training data includes the added compound; and retraining the machine learning model using the set of training data, wherein the set of training data includes shared molecular representations and respective property differences of each pair of molecules of the second set of molecule pairs of the set of training data. In another aspect, the compound paired with the identified compound has a property improvement greater than other compounds paired with the identified compound in the learning dataset. In another aspect, creating the set of molecule pairs using each molecule of the set of training data, further comprises: splitting the set of training data into one or more sets selected from the group consisting of: a training set, a test set, and the learning dataset. In another aspect, creating the set of molecule pairs using each molecule of the set of training data, further comprises: generating a pair of molecules for at least one of the training set, the test set, and the learning dataset by cross merging a first molecule and a second molecule of the training set, the test set, or the learning dataset, wherein all possible molecule pairs of the training set, the test set, or the learning dataset are generated and the learning set is cross merged with one molecule of the training set, wherein cross merging of the training set is limited to molecules of the training set, wherein cross merging of the test set is limited to molecules of the test set, and wherein cross merging of the learning set is limited to the one molecule of the training set and molecules of the learning set, wherein the one molecule of the training set includes a desired property value. In one aspect, the method further comprises: for a pair of molecules from an external dataset, predicting a property difference of the pair of molecules from the external dataset using the machine learning model as trained based on property differences of each pair of molecules of the set of training data.

Another embodiment described herein is a computer program product comprising program instructions stored on a machine-readable storage medium, wherein when the program instructions are executed by a computer processor, the program instructions cause the computer processor to execute the method as described herein.

Another embodiment described herein is a computer-implemented method for training a machine learning model for predicting which of a pair of molecules has an improved property value, the method comprising: receiving a set of data including molecules, wherein each molecule of the set of data includes a molecular representation and a value selected from a group consisting of: a known exact absolute property value and a known bound absolute property value, wherein the known exact absolute property value and the known bound absolute property value are related to a property of each molecule of the set of data; creating a set of training data with the set of data; creating a set of molecule pairs using each molecule of the set of training data; filtering the set of training data based on a set of rules, wherein the filtered set of training data includes molecule pairs of the set of molecule pairs having at least one molecule with a property value improved compared to the other molecule; training a machine learning model of an AI system using datapoints of the filtered set of training data, wherein the datapoints include molecular pairs of the filtered set of training data with shared representations, and wherein the datapoints include at least one selected from the group consisting of: bounded datapoints and exact regression datapoints; and for datapoints of a pair of molecules, predicting a property value improvement of molecular derivatization using the machine learning model as trained based on property differences of the datapoints, wherein the property value improvement indicates at least one molecule of the pair of molecules includes a property value greater than the other molecule. In one aspect, filtering the set of training data based on the set of rules, further comprises: removing, from the set of training data, molecular pairs of the set of training data with a property difference below a property difference threshold value. In another aspect, filtering the set of training data based on the set of rules, further comprises: removing, from the set of training data, molecular pairs of the set of training data having a first molecule and a second molecule with equal property values. In another aspect, filtering the set of training data based on the set of rules, further comprises: removing, from the set of training data, molecular pairs of the set of training data when an improved property is unknown. In another aspect, creating the set of molecule pairs using each molecule of the set of training data, further comprises: splitting the set of training data into a training set and a test set. In another aspect, creating the set of molecule pairs using each molecule of the set of training data, further comprises: generating a pair of molecules for at least one of the training set and the test set by cross merging a first molecule and a second molecule of the training set and the test set, wherein all possible molecule pairs of the training set and the test set are generated, wherein cross merging of the training set is limited to molecules of the training set, and wherein cross merging of the test set is limited to molecules of the test set.

Another embodiment described herein is a computer program product comprising program instructions stored on a machine-readable storage medium, wherein when the program instructions are executed by a computer processor, the program instructions cause the computer processor to execute the method as described herein.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed.

Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### EXAMPLES

### Example 1

There are several related, powerful approaches to predict property differences between two molecules, but they have important shortcomings that limit their broad practical deployment. For example, one of the most powerful approaches to predict property differences between two molecules is Free Energy Perturbations (FEP), with promising results in ab initio molecular optimization. However, FEP calculations are prohibitively complex and resource intensive, which hinders their broad deployment. Although “DeltaDelta” neural networks have emerged to predict binding affinity differences for two molecules more rapidly than previous algorithms, their use of protein-ligand complexes as input requires costly structural biology. Conversely, Matched Molecular Pair (MMP) analysis allows the rapid anticipation of property differences but can only predict differences between close molecular derivatives, is limited to common molecular derivations, and can fail to account for important chemical context.

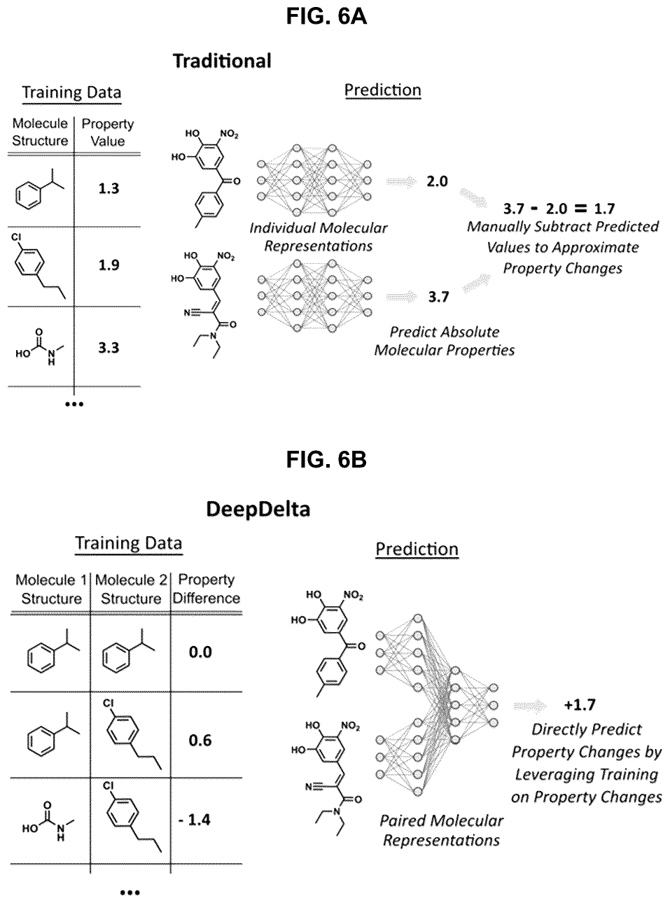

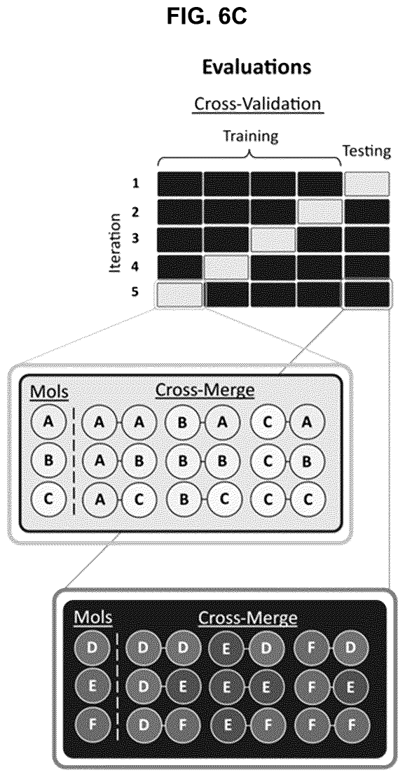

Here, we evaluate the potential of two state-of-the-art molecular machine learning algorithms, classic Random Forest models and the message passing neural network ChemProp, to predict ADMET property differences between two molecular structures. We chose Random Forest to represent classical machine learning methods given its robust performance for molecular machine learning tasks and chose ChemProp to represent deep learning methods as it leverages a hybrid representation of convolutions centered on bonds and exhibits strong predictive power for a range of molecular property benchmark datasets. Both methods show mediocre resolution to correctly predict property differences, limiting their utility for molecular optimization tasks. Motivated by this shortcoming, we propose DeepDelta, which directly learns property differences for pairs of molecules (FIG. 6B). DeepDelta shows significantly improved performance in most (82% in terms of Pearson's r and 73% in terms of MAE) of our benchmarks, including on all external test datasets. We analyze additional properties of DeepDelta from first mathematical principles, which enables us to derive accurate and rapidly calculatable confidence measures that are predictive of the model's performance. In contrast to existing molecular comparison approaches such as FEP and MMP, our DeepDelta approach can rapidly predict property differences between millions of chemically unrelated molecular pairs while accounting for molecular context without requiring complex ab initio calculations or protein-ligand complexes. Taken together, we believe that DeepDelta and extensions thereof will enable more accurate and holistic prioritization of drug lead series and thereby enable computation to support drug development more productively.

### Datasets

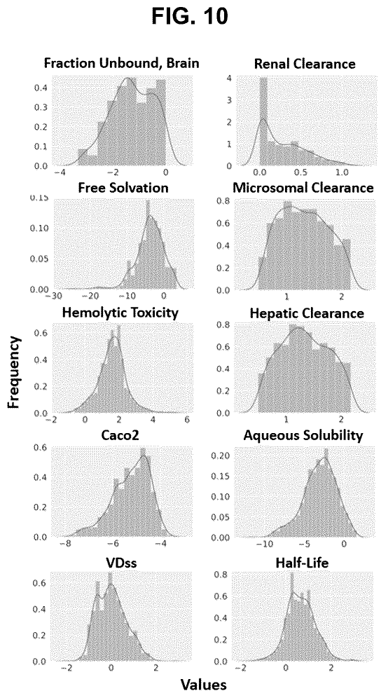

We extracted 10 publicly available datasets of various ADMET properties primarily from the Therapeutics Data Commons (Table 1). Invalid SMILES were removed from all datasets except for “Hemolytic Toxicity,” in which incorrectly notated amine groups were manually corrected based on original literature sources. Datapoints originally annotated as “>” or “<” instead of “=” were removed. We log-transformed all datasets except for the “FreeSolv dataset,” in which negative values prohibit log-transformation. For the renal clearance dataset, we incremented all annotated values by one to avoid values of zero during log-transformation. Distributions of transformed values for all datasets are shown in FIG. 10.

TABLE 1
Benchmarking Datasets

| Index | Property | Size | Units | Original Source |
|---|---|---|---|---|
| 1 | Fraction Unbound, Brain | 253 | Log (fu, brain) | Esaki, T., Journal of Chemical |
| | | | | Information and Modeling, |
| | | | | 59(7): 3251-3261 (2019). |
| 2 | Renal Clearance | 636 | Log(CLr) | Chen, J., Chemical Research in |
| | | | | Toxicology, 33(2): 640-650 |
| | | | | (2020). |
| | | | Experimental | Mobley, D. L., Journal of |
| | | | | Computer-aided Molecular |
| 3 | Free Solvation | 642 | Hydration Free Energy | Design, 28(7): 711-720 (2014). |
| | | | in Water | Di, L., European journal of |
| 4 | Microsomal Clearance | 731 | Log(mL/min/kg | Medicinal Chemistry, 57: 441- |
| | | | cleared) | 448 (2012). |
| 5 | Hemolytic Toxicity | 828 | Log(HD50) | Zheng, S., Journal of Chemical |
| | | | | Information and Modeling, |
| | | | | 60(6): 3231-3245 (2020). |
| 6 | Hepatic Clearance | 881 | Log(mL/min/kg | Di, L., European journal of |
| | | | cleared) | Medicinal Chemistry, 57: 441- |
| | | | | 448 (2012). |
| | | | | Wang, N. N., Journal of |
| 7 | Caco2 | 910 | Log(Papp) | Chemical information and |
| | | | | Modeling, 56(4): 763-773 |
| | | | | (2016). |
| 8 | Aqueous Solubility | 1128 | LogS | Delaney, J. S., Drug discovery |
| | | | | today, 10(4), 289-295 (2005). |
| | | | | Lombardo, F., Journal of |
| 9 | Volume of Distribution at | 1130 | Log(Body/Blood | Chemical Information and |
| | Steady State | | Concentration in L/kg) | Modeling, 56(10): 2042-2052 |
| | | | | (2016). |
| 10 | Half-Life | 1321 | Log(Half-Life in Hours) | Lombardo, F., Drug Metabolism |
| | | | | and Disposition, 46(11), 1466- |
| | | | | 1477 (2018). |

External test sets were collected from primary literature sources using the ChEMBL database to identify suitable publications. All invalid SMILES were removed. All datapoints annotated as “>” or “<” instead of “=” were removed. Datapoints in the external datasets that are also present in the training data were identified and removed based on Tanimoto similarity using Morgan Fingerprint (radius 2, 2048 bits, RDKit version 2022.09.5, threshold of 1.0 to remove identical molecules). Datapoint values in the external test sets were log-transformed to match training data while removing any datapoints with an initial value of 0.

### Model Architecture and Implementation

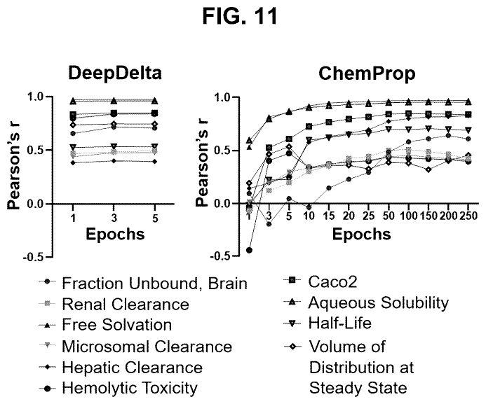

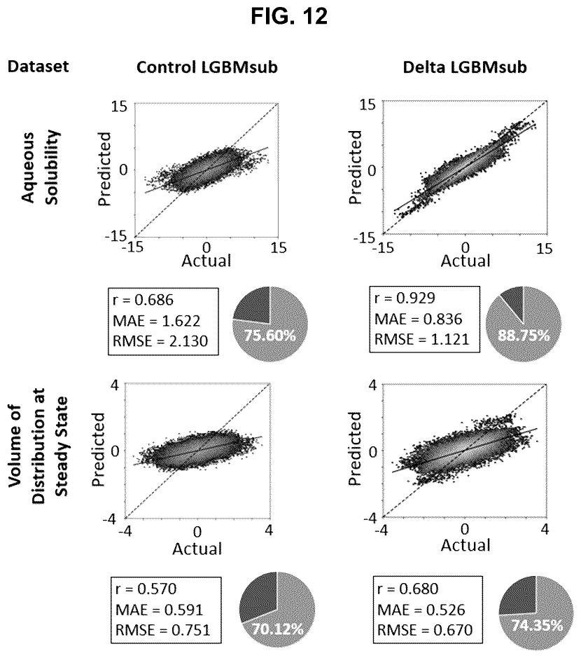

To develop DeepDelta, we used the same underlying D-MPNN architecture as ChemProp given its efficient computation and its competitive performance on molecular data. Furthermore, by building on this architecture, our results become easily comparable to the ChemProp implementation and allow us to directly quantify the benefit of our molecular pairing approach. Two molecules form an input pair for DeepDelta, while ChemProp processes a single molecule to predict absolute property values that are then subtracted to calculate property differences between two molecules. By training on input pairs and their property differences, DeepDelta directly learns and predicts property changes instead of requiring manual subtraction of predicted properties to approximate property changes. For ChemProp and DeepDelta, molecules were described using atom and bond features as previously implemented. In short, molecular graphs are converted into a latent representation by passing through a D-MPNN. For DeepDelta, this is done separately for each molecule and the latent representations of both molecules are subsequently concatenated. The concatenated embedding is then passed through a second neural network for property prediction that consists of linear feed forward layers. Both deep learning models were implemented for regression with default parameters and aggregation=‘sum’ using the PyTorch deep learning framework. For the traditional ChemProp implementation, number_of_molecules=1 while for DeepDelta number_of_molecules=2 to allow for processing of multiple inputs. We optimized the number of epochs for every model and set epochs=5 for DeepDelta and epochs=50 for ChemProp (FIG. 11).

For Random Forest and Light Gradient Boosting Machine (LightGBM, Microsoft) models, molecules were described using radial chemical fingerprints (Morgan Fingerprint, radius 2, 2048 bits, rdkit.org). The Random Forest regression machine learning models with 500 trees were implemented with default parameters in scikit-learn. The LightGBM was implemented with a subsample frequency of 0.1 to further improve running time on large datasets (LGBMsub) and otherwise default parameters, except for in the “Fraction Unbound, Brain” dataset, where we used min_child_samples=5 due to the small size of the original dataset. For traditional implementations of Random Forest and LightGBM, each molecule was processed individually (i.e., predictions are made solely based on the fingerprint of a single molecule), and property differences are calculated by making two separate predictions (one for each molecule) and these predictions are subsequently subtracted to calculate property differences between two molecules. For the delta version of LightGBM, fingerprints for paired molecules were concatenated to form paired molecular representations to directly train on and predict property changes. LightGBM models were implemented to evaluate pairwise methods applied to classic tree-based machine learning methods due to LightGBM's increased efficiency in handling large datasets compared to other tree-based methods.

### Model Evaluation

Models were evaluated using 5×10 fold cross-validation (sklearn), and performance was measured using Pearson's r, MAE, and root mean squared error (RMSE). To prevent data leakage, training data was first split into train and test sets during cross-validation prior to cross-merging to create molecule pairings (FIG. 6C); i.e., every molecule will only be present in pairs made from either the training or the test set but not both. Through this method, all possible pairs within a set are made. Additionally, the order of molecules matters, preserving both the magnitude and direction of property changes. Plots of cross-validation results were made with matplotlib from cross-validation splits with a random state=1. MMP analysis was implemented in KNIME using nodes from the RDKit and Vernalis community extensions. SMILES were preprocessed by de-salting, removing explicitly defined stereocenters and double bond geometries, canonicalizing, and filtering duplicates. Following fragmentation, matched molecular pairs were identified and grouped together using the canonical SMILES. Scaffold analysis and comparisons of Tanimoto similarity, Delta values, and errors were made with cross-validation splits with a random state=1 and plotted with matplotlib. Analysis of additional properties of DeepDelta were made with cross-validation splits with a random state=1. Paired t-tests were performed for comparison of the five repeats of our ten-fold cross-validation and the Kolmogorov-Smirnov test was performed to assess normality of all distributions prior to comparisons with paired t-tests. Overall comparisons of performance across benchmarks were assessed using the non-parametric Wilcoxon signed-rank tests. Code and data for all these calculations can be found at github.com/RekerLab/DeepDelta.

### Performance of Established Approaches

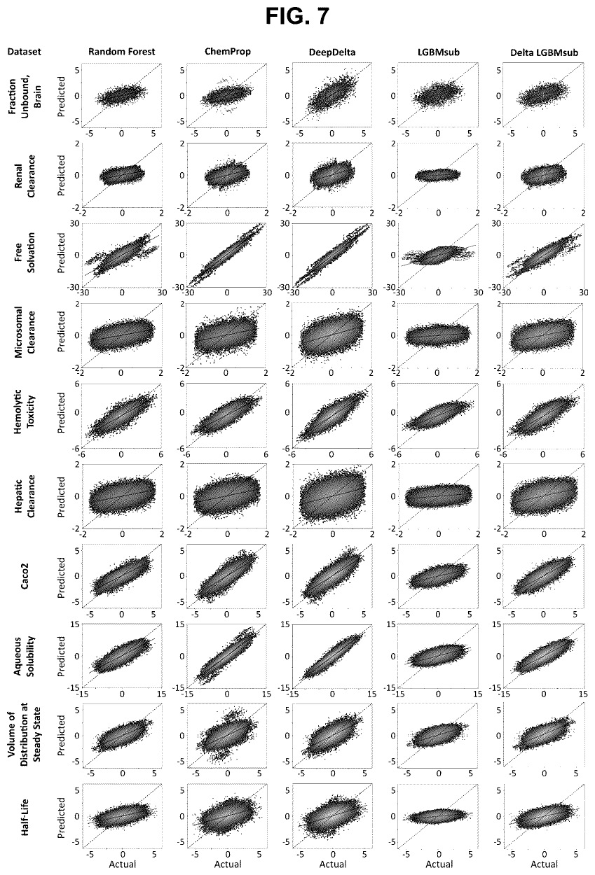

We first investigated whether established classic machine learning (Random Forest using circular fingerprints) and graph-based deep learning (ChemProp) algorithms could be used to predict differences in ADMET properties between two molecular structures. For this, we split all our benchmark datasets randomly into training and testing sets following a cross-validation strategy. The models (Random Forest or ChemProp) were then trained on the training fold and used to predict the properties of the molecules in the testing fold. Instead of directly evaluating the predicted property values of the test set molecules against the annotated ground truth, as is usually done, we evaluated the ability of our models to predict relative property differences between all possible pairs of molecules in the test set by subtracting their predicted property values and comparing these differences to the subtracted ground truth property values (FIG. 6A). In other words, absolute properties of individual molecules were predicted using individual molecular representations, and the predicted values were then subsequently subtracted to approximate molecular differences, meaning the models are not directly predicting property differences. We found overall mediocre performance of these established machine learning algorithms to predict property differences with median Pearson's r values across all benchmarking datasets of 0.60 for ChemProp and 0.63 for the Random Forest models (FIG. 7 and Table 3). This limited performance illuminates an opportunity for novel machine learning approaches tailored to predict property differences between molecules to improve our predictive power and resolution for molecular optimizations. Of note, we also explored the option of using MMP on these benchmark datasets, but standard MMP implementations can only make predictions for 0.6% of the molecular pairs in our data, highlighting the necessity of a more broadly applicable approach.

### DeepDelta Improves Performance

We hypothesized that a neural network specifically trained to predict property differences could potentially outperform established machine learning models on this task. To test this, we generated a new machine learning task in which every datapoint was composed of a pair of molecules and the objective variable is the difference in their properties (FIG. 6B). This data serves as input to a deep learning model that accepts two molecules as inputs and predicts the property difference between these molecules. This new approach, retrospectively tested on all our benchmark datasets using the same cross-validation scheme, significantly outperformed Random Forest and ChemProp on the level of the individual benchmarks (p=0.006) and achieved a promising, higher median Pearson's r of 0.72 (Table 2). Through the combinatorial expansion of training data resulting from pairing, DeepDelta also converged immediately while implementations of deep models that process a single molecule to predict absolute property values typically require training for multiple epochs to converge when used on small datasets (FIG. 11). The rapid convergence and improved performance of the DeepDelta approach over the standard implementation of ChemProp highlights how this method can allow smaller datasets (<1500 datapoints) to be more effectively processed by deep learning methods that are more data hungry.

When comparing the performance of DeepDelta to ChemProp or Random Forest models on the level of individual benchmarks (FIG. 7), DeepDelta performed similar or better in 90% of the benchmarks when considering Pearson's r. DeepDelta showed the most pronounced improvement for the “Fraction Unbound, Brain” dataset with improvements of at least 0.17 according to Pearson's r and a MAE reduction of at least 0.07 compared to other models. While improvements were less pronounced in other datasets, DeepDelta still statistically outcompeted ChemProp in 70% of datasets (p<0.05) and Random Forest in 70% of datasets (p<0.05) for Pearson's r with no significant change compared to each control model for 2 of the remaining datasets. DeepDelta exhibited a moderate but significant average improvement in Pearson's r across all datasets of 0.04 (p=0.006), with a maximum improvement of 0.22. DeepDelta also outcompeted 60% of the benchmarks in terms of MAE (Table 2) and exhibited a small, but significant average improvement in MAE across all datasets of 0.13 (p=0.04), with a maximum improvement of 1.063. It is worth noting that all applied models showed poor performance (Pearson's r<0.5) on the three datasets related to clearance and only moderate predictivity for half-life, possibly driven by the complexity of predicting clearance from the molecular structure alone when provided with limited data that does not fully capture all the different elimination pathways for a specific tissue. In particular, “Hepatic Clearance” is the only benchmarking dataset where the DeepDelta approach is significantly outperformed by the other models in terms of Pearson's r. In the future, we expect increasing amounts of data for specific elimination pathways to enable better predictions for all models for such tasks and to particularly benefit DeepDelta to more accurately capture differences in elimination between two structures. Already, the competitive performance of our pairing approach compared to established approaches highlights the ability of DeepDelta to improve performance of machine learning for current datasets of ADMET properties with large potential for further development.

### Tree-Based Delta Approach

To further evaluate whether our new paired machine learning task could also be solved by classic tree-based machine learning methods, we implemented Microsoft's Light Gradient Boosting Machine (LightGBM) that we parametrized to subsample the training data for more efficient training on large datasets (LGBMsub). Analogously to the training of DeepDelta, we provided the Delta LGBMsub models with a representation of both molecules by concatenating Morgan circular fingerprints of the two molecules and trained them on property differences between the two molecules. Compared to the performance of the regular LGBMsub models (i.e., trained on individual molecules and calculating predicted differences by subtracting predictions analogously to FIG. 6A), the paired Delta LGBMsub models showed significant improvement in Pearson's r, MAE, and RMSE across all benchmark datasets during retrospective cross-validations (FIG. 6, Table 2). These data suggest that the paired machine learning task can improve the performance of classic machine learning algorithms when predicting property differences, but apparently to a lesser extent than the deep learning approach, as DeepDelta outperformed the paired Delta LGBMsub approach in all but one benchmark in terms of Pearson's r (p<0.05) and 60% of benchmarks in terms of MAE. The difference between the traditional LGBMsub and the paired Delta LGBMsub could be further reduced through parameter optimization and by reducing subsampling (Table 3). These results indicate that the molecular pairing approach can also be beneficial to tree-based architectures but appears most promising for deep neural networks where the combinatorial data explosion leads to significant performance improvements during cross-validation.

TABLE 2
Evaluations of 5 × 10-Fold Cross-Validation of Random Forest,
ChemProp, DeepDelta, LGBMsub, and Delta LGBMsub. Average and standard
deviation of Pearson's r, MAE, and RMSE are presented for all
5 models. Best performance per dataset (p < 0.05) is bolded

| | Pearson's r | | | | | | | |
|---|---|---|---|---|---|---|---|---|
| | | | | | Delta | Mean Absolute Error | | |
| | | Chem | Deep | LGBM | LGBM | | Chem | Deep |
| Dataset | RF | Prop | Delta | sub | sub | RF | Prop | Delta |
| Fraction | 0.535 ± | 0.483 ± | 0.701 ± | 0.47 ± | 0.53 ± | 0.713 ± | 0.740 ± | 0.642 ± |
| Unbound, | 0.008 | 0.023 | 0.013 | 0.041 | 0.015 | 0.008 | 0.01 | 0.014 |
| Brain | | | | | | | | |
| Renal | 0.461 ± | 0.478 ± | 0.465 ± | 0.283 ± | 0.443 ± | 0.256 ± | 0.262 ± | 0.269 ± |
| Clearance | 0.01 | 0.013 | 0.023 | 0.016 | 0.007 | 0.002 | 0.004 | 0.006 |
| Free | 0.843 ± | 0.967 ± | 0.971 ± | 0.589 ± | 0.898 ± | 1.874 ± | 0.937 ± | 0.806 ± |
| Solvation | 0.005 | 0.0 | 0.001 | 0.007 | 0.003 | 0.028 | 0.006 | 0.003 |
| Microsomal | 0.444 ± | 0.451 ± | 0.468 ± | 0.273 ± | 0.42 ± | 0.434 ± | 0.436 ± | 0.444 ± |
| Clearance | 0.002 | 0.011 | 0.007 | 0.011 | 0.01 | 0.002 | 0.003 | 0.002 |
| Hemolytic | 0.821 ± | 0.778 ± | 0.842 ± | 0.706 ± | 0.816 ± | 0.506 ± | 0.566 ± | 0.487 ± |
| Toxicity | 0.002 | 0.003 | 0.002 | 0.007 | 0.003 | 0.002 | 0.006 | 0.004 |
| Hepatic | 0.438 ± | 0.431 ± | 0.392 ± | 0.28 ± | 0.424 ± | 0.45 ± | 0.455 ± | 0.494 ± |
| Clearance | 0.004 | 0.005 | 0.007 | 0.013 | 0.013 | 0.002 | 0.002 | 0.003 |
| Caco2 | 0.829 ± | 0.851 ± | 0.853 ± | 0.565 ± | 0.829 ± | 0.472 ± | 0.451 ± | 0.444 ± |
| | 0.006 | 0.005 | 0.005 | 0.013 | 0.004 | 0.006 | 0.006 | 0.006 |
| Aqueous | 0.837 ± | 0.951 ± | 0.957 ± | 0.596 ± | 0.852 ± | 1.237 ± | 0.687 ± | 0.644 ± |
| Solubility | 0.003 | 0.001 | 0.001 | 0.006 | 0.003 | 0.003 | 0.005 | 0.006 |
| Volume of | 0.728 ± | 0.697 ± | 0.746 ± | 0.578 ± | 0.719 ± | 0.483 ± | 0.505 ± | 0.470 ± |
| Distribution | 0.003 | 0.003 | 0.005 | 0.006 | 0.003 | 0.003 | 0.004 | 0.004 |
| at | | | | | | | | |
| Steady | | | | | | | | |
| State | | | | | | | | |
| Half-life | 0.529 ± | 0.508 ± | 0.534 ± | 0.330 ± | 0.514 ± | 0.586 ± | 0.600 ± | 0.597 ± |
| | 0.006 | 0.008 | 0.004 | 0.007 | 0.003 | 0.003 | 0.003 | 0.003 |

| | Mean Absolute Error | | Root Mean Squared Error | | | | |
|---|---|---|---|---|---|---|---|
| | | Delta | | | | | Delta |
| | LGBM | LGBM | | Chem | Deep | LGBM | LGBM |
| Dataset | sub | sub | RF | Prop | Delta | sub | sub |
| Fraction | 1.003 ± | 0.726 ± | 0.928 ± | 0.965 ± | 0.830 ± | 1.311 ± | 0.939 ± |
| Unbound, | 0.008 | 0.011 | 0.01 | 0.019 | 0.023 | 0.014 | 0.014 |
| Brain | | | | | | | |
| Renal | 0.307 ± | 0.265 ± | 0.337 ± | 0.339 ± | 0.350 ± | 0.396 ± | 0.344 ± |
| Clearance | 0.003 | 0.002 | 0.002 | 0.005 | 0.008 | 0.004 | 0.002 |
| Free | 4.23 ± | 1.656 ± | 2.914 ± | 1.372 ± | 1.290 ± | 5.472 ± | 2.415 ± |
| Solvation | 0.058 | 0.027 | 0.045 | 0.009 | 0.023 | 0.064 | 0.037 |
| Microsomal | 0.509 ± | 0.443 ± | 0.544 ± | 0.546 ± | 0.557 ± | 0.638 ± | 0.556 ± |
| Clearance | 0.004 | 0.002 | 0.001 | 0.005 | 0.002 | 0.005 | 0.003 |
| Hemolytic | 0.897 ± | 0.516 ± | 0.663 ± | 0.729 ± | 0.635 ± | 1.127 ± | 0.672 ± |
| Toxicity | 0.008 | 0.004 | 0.005 | 0.007 | 0.006 | 0.01 | 0.005 |
| Hepatic | 0.529 ± | 0.46 ± | 0.564 ± | 0.570 ± | 0.622 ± | 0.663 ± | 0.575 ± |
| Clearance | 0.006 | 0.005 | 0.002 | 0.002 | 0.004 | 0.008 | 0.005 |
| Caco2 | 0.82 ± | 0.473 ± | 0.614 ± | 0.575 ± | 0.572 ± | 1.036 ± | 0.61 ± |
| | 0.012 | 0.005 | 0.01 | 0.007 | 0.008 | 0.014 | 0.007 |
| Aqueous | 2.184 ± | 1.197 ± | 1.623 ± | 0.915 ± | 0.859 ± | 2.783 ± | 1.562 ± |
| Solubility | 0.029 | 0.004 | 0.011 | 0.008 | 0.012 | 0.038 | 0.011 |
| Volume of | 0.772 ± | 0.493 ± | 0.632 ± | 0.670 ± | 0.618 ± | 0.969 ± | 0.64 ± |
| Distribution | 0.006 | 0.003 | 0.003 | 0.004 | 0.006 | 0.008 | 0.003 |
| at | | | | | | | |
| Steady | | | | | | | |
| State | | | | | | | |
| Half-life | 0.662 ± | 0.594 ± | 0.762 ± | 0.784 ± | 0.778 ± | 0.849 ± | 0.771 ± |
| | 0.003 | 0.005 | 0.003 | 0.004 | 0.004 | 0.003 | 0.002 |

### Performance on External Data

We next investigated the generalizability of our new DeepDelta models by testing their performance on external test data. We sought external data for our three largest datasets, however, publicly available external datasets of appropriate size for “Half-life” overlapped with the training set or were derived through a different methodology (i.e., in vitro/in vivo animal assays instead of human clinical data). Therefore, we focused our external evaluation on “Solubility” and “Volume of Distribution at Steady State.” When training our models on our complete training data for these benchmarks and predicting pairs made exclusively from compounds in the external validation test sets, DeepDelta outperformed both Random Forest and ChemProp in all cases in terms of Pearson's r, MAE, and RMSE and in accuracy, defined as the percent of predictions correctly predicting a positive or negative property change (FIG. 7). Similarly, the paired LGBMsub approach showed improvements across all metrics on the external test sets compared to the regular LGBMsub (FIG. 11) but did not outperform DeepDelta. Together, these results highlight the potential for DeepDelta to support molecular optimization by accurately predicting effects on ADMET properties arising from chemical modifications even for compound pairs that originate from other datasets, suggesting that DeepDelta can effectively generalize and predict property differences between molecules outside of the training data.

### Mathematical Invariants

Apart from being able to make accurate predictions for property differences between two molecules, the pairing approach will also result in additional properties of our machine learning models. Specifically, an accurate DeepDelta model should capture the following three properties: predict zero property differences when provided the exact same molecule for both inputs,

$$DeepDelta ( x , x ) = 0 \qquad ( Eq . 1 )$$

predict the inverse of the original prediction when swapping the input molecules,

$$DeepDelta ( x , y ) = - D e e p D e l t a ( y , x ) \qquad ( Eq . 2 )$$

and preserve additivity for predicted differences between three molecules,

$$DeepDelta ( x , y ) + D e e p D e l t a ( y , z ) = D e e p D e l t a ( x , z ) \qquad ( Eq . 3 )$$

We analyzed our data to determine whether our DeepDelta models would adhere to these properties. For Eq. 1, we determined the MAE from 0 when DeepDelta predicted the change for pairs of the same molecule. For Eq. 2, we plotted predictions for all molecule pairs against the prediction of those pairs with their order reversed and determine their correlation (Pearson's r). For Eq. 3, we determined the MAE from 0 for the additivity of predicted differences for all possible groupings of three molecules. Gratifyingly, we observed that the DeepDelta models accurately captured these properties with overall low MAE (0.127±0.042) for the same molecule predictions (Eq. 1), strong anti-correlation (r=−0.947±0.044) for predictions with swapped inputs (Eq. 2), and overall low MAE (0.127±0.043) for the additive differences (Eq. 3) (Table 4). Notably, for same molecule predictions (Eq. 1) and additive differences (Eq. 3), the average MAE was over 4 times lower than cross-validation MAE—indicating that DeepDelta can learn these invariants more effectively than it can learn property differences between molecules (FIG. 9). Taken together, DeepDelta was able to accurately capture all three properties indicating it was able to learn basic principles of molecular changes.

### Model Performance

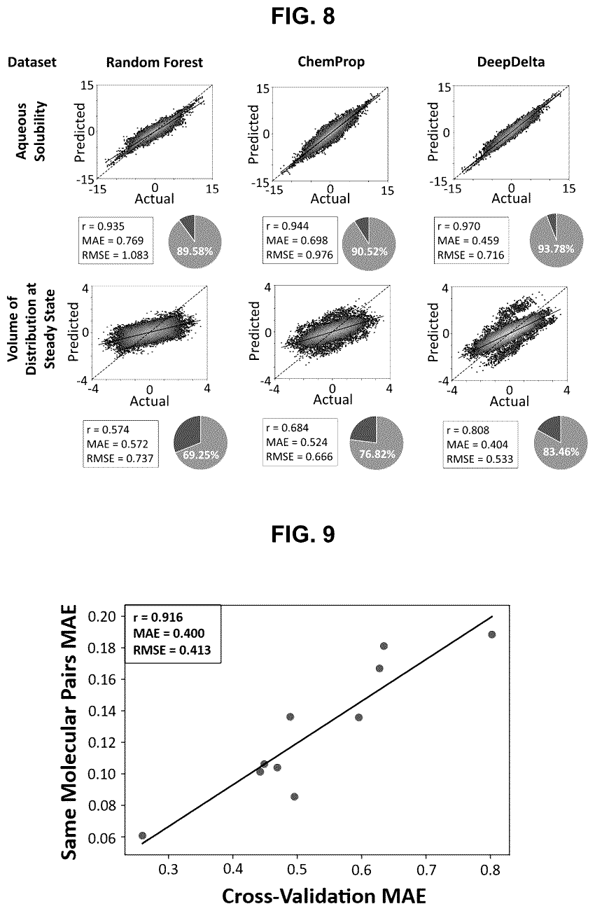

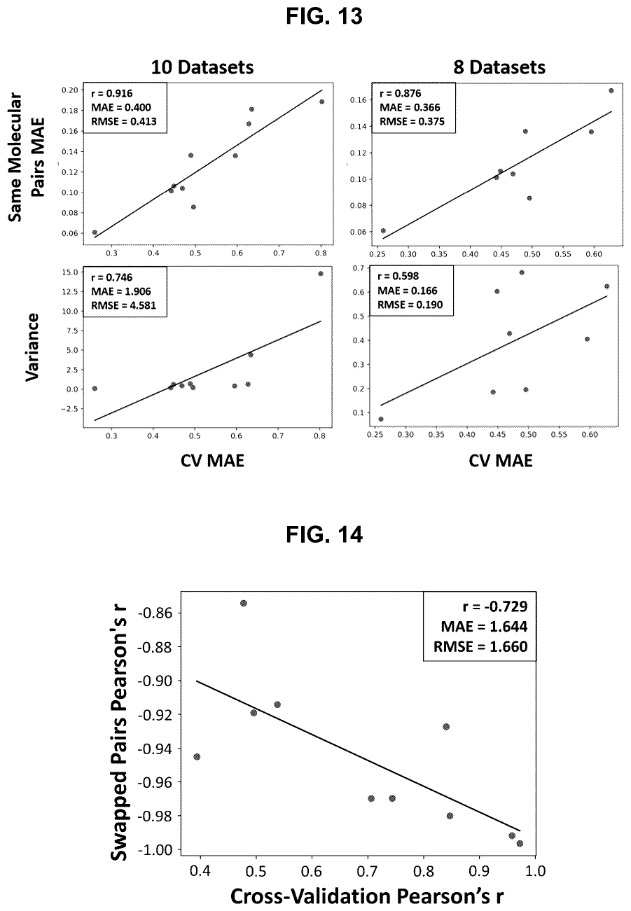

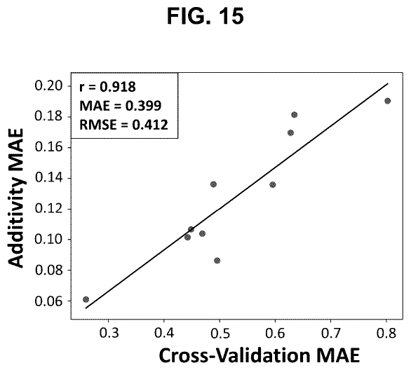

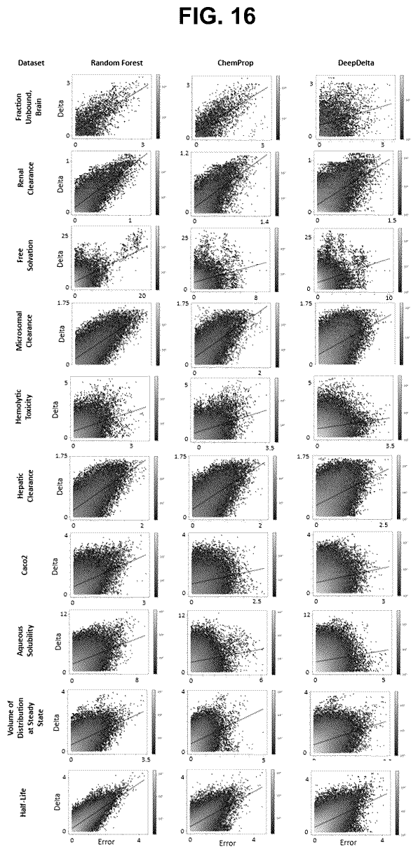

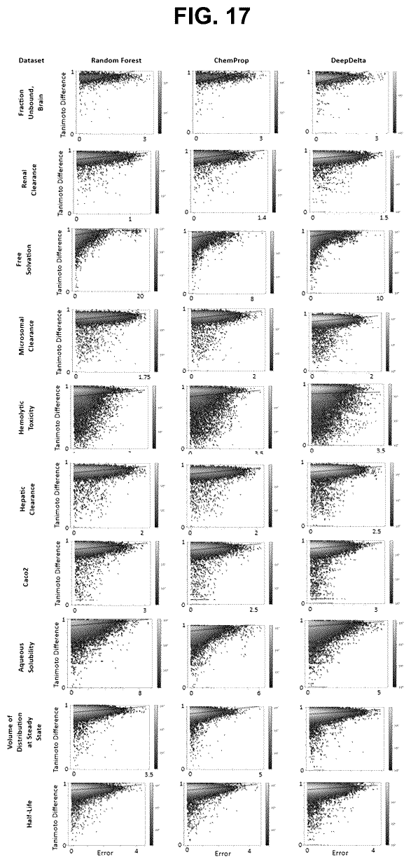

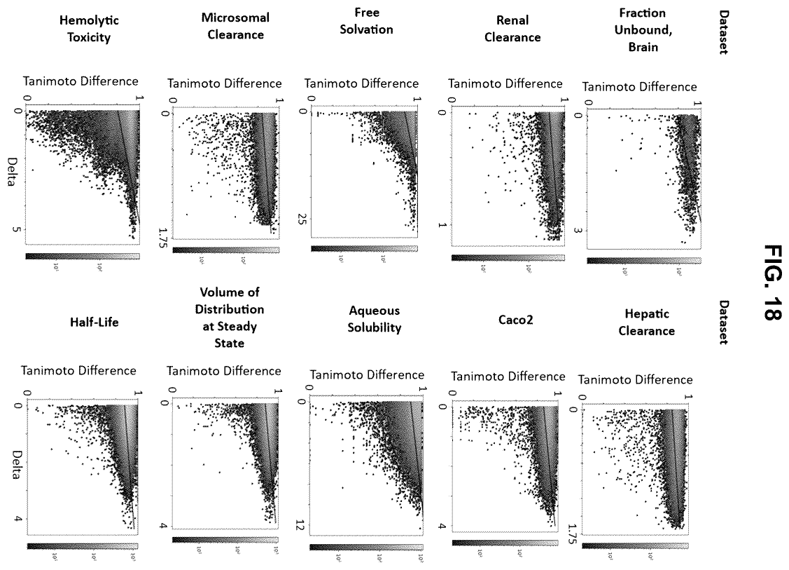

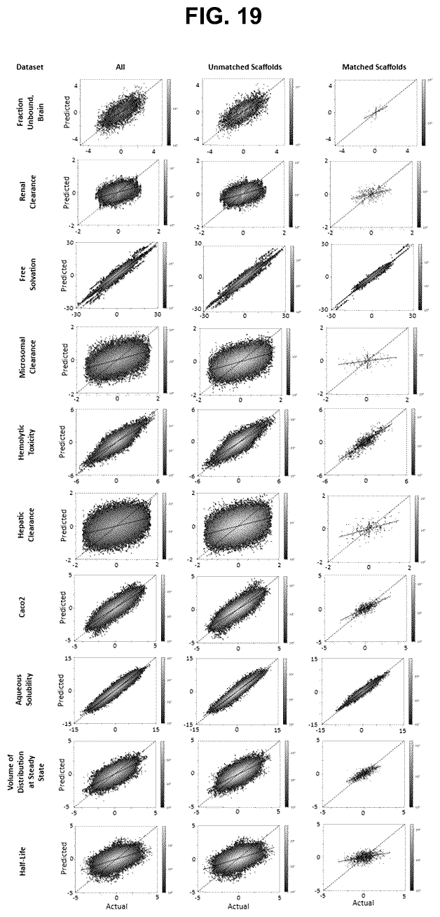

Although DeepDelta models trained on different datasets were overall compliant with the three properties of interest (i.e., equations 1-3), the performance of specific DeepDelta models on these mathematically fundamental tasks varied between datasets. We hypothesized that stronger performance on these tasks might correlate with overall performance of the DeepDelta models and thereby provide a measure of model convergence and applicability to a specific dataset. We evaluated whether (1) the MAE of same molecule predictions could predict the MAE of cross-validation performance, (2) the Pearson's r of the swapped inputs would be inversely related to the Pearson's r of the cross-validation, and (3) the MAE of additive differences would correlate with the MAE of the cross-validations. We found that a model's ability to correctly predict no change in property between the same molecules correlated strongly (r=0.916) with overall cross-validation performance (FIG. 8) and that this correlation was consistently stronger than that caused simply by the magnitude of variance found in the values across the datasets (r=0.746) and was maintained when outlier datasets with variance greater than 1 were removed (FIG. 13). Additionally, we observed that r values of the swapped inputs were inversely correlated with the r values from cross-validation (r=−0.729, FIG. 14) and the MAE values of additive differences were strongly correlated with the MAE from cross-validation (r=0.918, FIG. 15). Therefore, these mathematically fundamental calculations are indicative of the stability of the models and their overall performance. As these calculations can be performed on unlabeled data, this approach could serve as an indicator of how well a model will extrapolate to new chemical spaces.

### Predicting Large Property Differences

To further characterize the performance of our DeepDelta models, we next investigated whether the performance on individual predictions correlates with the magnitude of the observed property difference between the two molecules (FIG. 16, Table 5); i.e., whether it is easier for our models to correctly predict small property changes and harder for the models to accurately predict more drastic property differences. Across all datasets, DeepDelta predictions showed weak correlation between predictive error on individual datapoints and the absolute difference of properties between the two paired molecules (median Pearson's r of 0.3), but this correlation was stronger for the established ChemProp (median Pearson's r of 0.5) and Random Forest (median Pearson's r of 0.6) models. On the level of individual datasets, the correlation for DeepDelta was smaller in 9/10 datasets compared to ChemProp (p=0.01) and in 10/10 datasets compared to Random Forest (p=0.002), indicating that DeepDelta is capable of more accurately predicting larger property changes between two molecules compared to established models in all but one case. To further support this claim, we analyzed the error when predicting only the highest 10% of delta values (i.e., we evaluated only the molecular pairs with the largest difference in property values of the test fold) and observed that DeepDelta exhibited the lowest error for 10/10 datasets compared to Random Forest (p=0.002) and ChemProp (p=0.002). However, DeepDelta did exhibit the highest error rates for predicting the lowest 10% of delta values (p=0.002). This might potentially be driven by the loss function being less affected by errors on small property differences during model training, which could be improved in future model architectures specifically designed to predict small property differences. It is important to also note that these small property value differences lie well within experimental noise and variance and might therefore not be as reliable. Improved experimental resolution and automation should reduce noise and experimental error that may be common within the smallest molecular property deviations. At the same time, we did not observe a strong correlation between the chemical similarity of the molecules and the predictive error (FIG. 17), and this trend mimicked the distribution between chemical similarity and the ground-truth difference between the paired molecules for the property of interest (FIG. 18). This data highlights that DeepDelta outperforms established approaches particularly when predicting large property differences between distinct molecules, positioning it for challenging molecular optimization where large property changes are necessary.

### Scaffold-Hopping Potential

We next tested whether our DeepDelta model could more accurately predict pairs with the same or with different molecular scaffolds. To this end, we separated molecular pairs in the test-fold into two groups (pairs with the same scaffold or pairs with different scaffolds) and evaluated the performance of the model trained on the training-fold on both groups. DeepDelta predicted properties for pairs with differing Murcko scaffolds with similar accuracy (p=0.11) compared to pairs with the same scaffold (FIG. 19, Table 6), indicating this method is robust to major structural alterations. Although ChemProp and Random Forest also showed good performance for molecules with differing scaffolds, DeepDelta outperformed both models when predicting molecular pairs with distinct scaffolds with a moderate but significant average improvement of 0.04 in terms of Pearson's r (p=0.004, Table 6) and a small, but significant average improvement of 0.04 in terms of MAE (p=0.01, Table 6). On the level of individual datasets, DeepDelta shows improvement over ChemProp in 8/10 datasets and Random Forest in 9/10 datasets in terms of Pearson's r, altogether indicating that DeepDelta has potential to guide molecular optimizations that involve scaffold hopping. This better performance at scaffold hopping does not make DeepDelta worse at predicting changes between molecules sharing the same scaffold compared to Random Forest or ChemProp, as DeepDelta showed statistically indistinguishable performance to these models both in terms Pearson's r (p>0.3) and MAE (p>0.1), meaning DeepDelta presents itself as the model of choice to enable optimization of compounds within the same scaffold as well as to perform scaffold hoping.

We here conceived, implemented, validated, and characterized DeepDelta, a novel deep machine learning approach that allows for direct training on and prediction of property differences between two molecules. Given the importance of ADMET property optimization for drug development, we here specifically tested our method for 10 established ADMET property benchmarking datasets. These are challenging tasks for molecular machine learning given the complexity of the modeled processes, which often involves intricate tissue interactions of molecules, and the small dataset sizes, commonly derived from low-throughput in vivo experiments. Our approach, DeepDelta, outperforms the established, state-of-the-art molecular machine learning models ChemProp and Random Forest for predicting property differences between molecules in the majority of our benchmarks (82% for Pearson's r and 73% for MAE), including all external test datasets. DeepDelta represents, to the best of our knowledge, the first attempt to directly train machine learning models to predict molecular property differences.

DeepDelta appears particularly powerful when predicting larger property changes (FIG. 17) and can also predict differences between molecules with different scaffolds more effectively (FIG. 19), indicating that DeepDelta might be particularly suitable to optimize compounds with drastic ADMET liabilities that might benefit from scaffold hopping into new compound classes. Competitive performance within the same scaffold class indicates that DeepDelta is equally applicable for more fine-grained optimization. DeepDelta benefits from directly learning property difference and data augmentation that increases training datapoints for deep neural networks while also cancelling systematic errors within datasets through pairing. However, pairwise methods like DeepDelta have increased computational costs for model training given the combinatorial expansion of training data sets. As such, we believe these methods are optimally suited for smaller datasets (<1500 datapoints) and provide the benefit of allowing these smaller datasets to be appropriately applied to data-hungry deep learning models.

Several other molecular pairing approaches have been deployed for various purposes. For example, the pairwise difference regression (PADRE) approach trains machine learning models on pairs of feature vectors to improve the predictions of absolute property values and their uncertainty estimation. PADRE similarly benefits for combinatorial expansion of data; however, PADRE predicts absolute values of unseen molecules like traditional methods instead of being tailored for prediction of property differences. Similarly, Lee and colleagues have used pairwise comparisons to allow for use of qualitative measurements with quantitative ones and AstraZeneca has created workflows that utilize compound pairs to train Siamese neural networks to classify the bioactivity of small molecules. These classification-based methods can allow for direct handling of truncated values through Boolean comparisons. In contrast, the regression-based DeepDelta provides a means of quantifying molecular differences. In computational chemistry, A-Machine Learning approaches aim to accelerate and improve quantum property computations by using machine learning to anticipate property differences to a baseline. We believe that existing molecular pairing approaches deployed for other purposes will be synergistic with our DeepDelta approach and have the potential to augment or replace standard molecular machine learning approaches for intricate optimization and discovery tasks, especially for complex properties and small datasets.

An intriguing property of DeepDelta is its ability to adhere to mathematical invariants, such as the prediction of zero changes when inputting the same molecule (Eq. 1), the expected inverse relationships when molecule order was inverted (Eq. 2), and the additivity of the predicted differences (Eq. 3)—all of which indicate the models were able to learn basic principles of molecular changes. Interestingly, the performance of the models on these tasks correlated strongly with overall cross-validation performance (FIG. 8), suggesting that such unsupervised calculations could be indicative of model performance and convergence and thereby allow for increased transparency and determination of model applicability to specific datasets. For example, one could evaluate DeepDelta performance on the invariant calculations across a number of new datasets as a predictor of how the DeepDelta approach would likely perform on these datasets to prioritize the datasets on which to apply DeepDelta.

Taken together, we believe that DeepDelta and extensions thereof will provide accurate and easily deployable predictions to steer molecular optimization and compound prioritization. We have here shown its applicability to ADMET property comparison, which is of particular importance to drug development to ensure safety and efficacy of medications but notoriously difficult to predict given the complexity of the involved biological processes and the small datasets resulting from complex in vivo experiments. DeepDelta may effectively guide molecular optimization by informing a project team on the most promising candidates to evaluate next or could be directly integrated into automated, robotic optimization platforms to create safer and more effective drug leads through iterative design. Beyond drug development, we expect DeepDelta to also benefit other tasks in biological and chemical sciences to de-risk material optimization and selection.

TABLE 3
Parameter Optimizations during 5 × 10-Fold Cross-Validation of LGBM Traditional,
and LightGBM Delta. Average and standard deviation of Pearson's r, mean absolute
error (MAE), and root mean squared error (RMSE) are presented for all 3 models.

| | Pearson's r | | | MAE | | | RMSE | | |
|---|---|---|---|---|---|---|---|---|---|
| | LGBM | LGBM | LGBM | LGBM | LGBM | LGBM | LGBM | LGBM | LGBM |
| Dataset | Trad | Trad | Delta | Trad | Trad | Delta | Trad | Trad | Delta |
| Subsample | 0.1 | 1 | 0.1 | 0.1 | 1 | 0.1 | 0.1 | 1 | 0.1 |
| Frequency | | | | | | | | | |
| Fraction | 0.47 ± | 0.553 ± | 0.53 ± | 1.003 ± | 0.941 ± | 0.726 ± | 1.311 ± | 1.231 ± | 0.939 ± |
| Unbound, | 0.041 | 0.02 | 0.015 | 0.008 | 0.016 | 0.011 | 0.014 | 0.024 | 0.014 |
| Brain | | | | | | | | | |
| Renal | 0.283 ± | 0.453 ± | 0.443 ± | 0.307 ± | 0.36 ± | 0.265 ± | 0.396 ± | 0.458 ± | 0.344 ± |
| Clearance | 0.016 | 0.011 | 0.007 | 0.003 | 0.004 | 0.002 | 0.004 | 0.005 | 0.002 |
| Free | 0.589 ± | 0.83 ± | 0.898 ± | 4.23 ± | 4.283 ± | 1.656 ± | 5.472 ± | 5.506 ± | 2.415 ± |
| Solvation | 0.007 | 0.005 | 0.003 | 0.058 | 0.034 | 0.027 | 0.064 | 0.067 | 0.037 |
| Microsomal | 0.273 ± | 0.409 ± | 0.42 ± | 0.509 ± | 0.581 ± | 0.443 ± | 0.638 ± | 0.732 ± | 0.556 ± |
| Clearance | 0.011 | 0.007 | 0.01 | 0.004 | 0.004 | 0.002 | 0.005 | 0.004 | 0.003 |
| Hemolytic | 0.706 ± | 0.832 ± | 0.816 ± | 0.897 ± | 0.956 ± | 0.516 ± | 1.127 ± | 1.231 ± | 0.672 ± |
| Toxicity | 0.007 | 0.003 | 0.003 | 0.008 | 0.004 | 0.004 | 0.01 | 0.003 | 0.005 |
| Hepatic | 0.28 ± | 0.418 ± | 0.424 ± | 0.529 ± | 0.625 ± | 0.46 ± | 0.663 ± | 0.786 ± | 0.575 ± |
| Clearance | 0.013 | 0.005 | 0.013 | 0.006 | 0.004 | 0.005 | 0.005 | 0.005 | 0.005 |
| Caco2 | 0.565 ± | 0.827 ± | 0.829 ± | 0.82 ± | 0.881 ± | 0.473 ± | 1.036 ± | 1.118 ± | 0.61 ± |
| | 0.013 | 0.006 | 0.004 | 0.012 | 0.009 | 0.005 | 0.014 | 0.013 | 0.007 |
| Aqueous | 0.596 ± | 0.85 ± | 0.852 ± | 2.184 ± | 2.397 ± | 1.197 ± | 2.783 ± | 3.02 ± | 1.562 ± |
| Solubility | 0.006 | 0.002 | 0.003 | 0.029 | 0.019 | 0.004 | 0.038 | 0.022 | 0.011 |
| Volume of | 0.578 ± | 0.73 ± | 0.719 ± | 0.772 ± | 0.778 ± | 0.493 ± | 0.969 ± | 0.984 ± | 0.64 ± |
| Distribution at | 0.006 | 0.004 | 0.003 | 0.006 | 0.004 | 0.003 | 0.008 | 0.006 | 0.003 |
| Steady State | | | | | | | | | |
| Half-life | 0.330 ± | 0.516 ± | 0.514 ± | 0.662 ± | 0.598 ± | 0.594 ± | 0.849 ± | 0.776 ± | 0.771 ± |
| | 0.007 | 0.011 | 0.003 | 0.003 | 0.006 | 0.005 | 0.003 | 0.006 | 0.002 |

TABLE 4
Evaluations of DeepDelta Models on Mathematical Invariants.
Mathematical invariants are compared against cross-
validation results (with random state = 1) and
analyzed with Pearson's r (r), mean absolute error
(MAE), and root mean squared error (RMSE).

| | Cross-Validation | | | Eq. 1 | Eq. 2 | Eq. 3 |
|---|---|---|---|---|---|---|
| Dataset | r | MAE | RMSE | MAE | r | MAE |
| Fraction | 0.7061 | 0.6278 | 0.812 | 0.1669 | −0.9699 | 0.1696 |
| Unbound, | | | | | | |
| Brain | | | | | | |
| Renal | 0.4955 | 0.2595 | 0.339 | 0.0608 | −0.9192 | 0.0609 |
| Clearance | | | | | | |
| Free Solvation | 0.9724 | 0.8024 | 1.271 | 0.1884 | −0.9966 | 0.1904 |
| Microsomal | 0.4774 | 0.4424 | 0.5544 | 0.1013 | −0.8543 | 0.1013 |
| Clearance | | | | | | |
| Hemolytic | 0.8404 | 0.4891 | 0.6411 | 0.1362 | −0.9274 | 0.1361 |
| Toxicity | | | | | | |
| Hepatic | 0.3938 | 0.4956 | 0.6234 | 0.0855 | −0.9452 | 0.0862 |
| Clearance | | | | | | |
| Caco2 | 0.8471 | 0.4487 | 0.5797 | 0.1062 | −0.9802 | 0.1065 |
| Solubility | 0.9585 | 0.6345 | 0.843 | 0.1811 | −0.9919 | 0.1814 |
| Volume of | 0.7438 | 0.469 | 0.6207 | 0.104 | −0.9698 | 0.1038 |
| Distribution at | | | | | | |
| Steady State | | | | | | |
| Half-life | 0.5378 | 0.5956 | 0.7726 | 0.1358 | −0.9143 | 0.1358 |

TABLE 5
Correlation (Pearson's r) of error and Property Differences between Paired
Datapoints following 5 × 10-Fold Cross-Validation Analysis

| Dataset | Random Forest | ChemProp | DeepDelta |
|---|---|---|---|
| Fraction Unbound, Brain | 0.698 | 0.707 | 0.274 |
| Renal Clearance | 0.731 | 0.634 | 0.544 |
| Free Solvation | 0.505 | 0.285 | 0.314 |
| Microsomal Clearance | 0.699 | 0.617 | 0.456 |
| Hemolytic Toxicity | 0.343 | 0.367 | 0.156 |
| Hepatic Clearance | 0.642 | 0.601 | 0.411 |
| Caco2 | 0.419 | 0.207 | 0.185 |
| Aqueous Solubility | 0.388 | 0.154 | 0.146 |
| Volume of Distribution at Steady State | 0.480 | 0.368 | 0.330 |
| Half-life | 0.736 | 0.591 | 0.516 |

TABLE 6
Evaluations of 10-Fold Cross-Validation of all Models for Matched
and Unmatched Scaffold Pairs. Pearson's r, mean absolute error
(MAE), and root mean squared error (RMSE) are listed as the 1st,
2nd, and 3rd number in each cell, respectively (random state = 1).

| | DeepDelta | | ChemProp | | Random Forest | |
|---|---|---|---|---|---|---|
| | Matched | Unmatched | Matched | Unmatched | Matched | Unmatched |
| Dataset | Scaffolds | Scaffolds | Scaffolds | Scaffolds | Scaffolds | Scaffolds |
| Fraction | 0.455 | 0.707 | 0.142 | 0.488 | 0.110 | 0.533 |
| Unbound, | 0.228 | 0.648 | 0.095 | 0.770 | 0.092 | 0.744 |
| Brain | 0.339 | 0.828 | 0.294 | 0.977 | 0.294 | 0.946 |
| | 0.388 | 0.497 | 0.434 | 0.504 | 0.485 | 0.466 |
| Renal | 0.142 | 0.262 | 0.098 | 0.259 | 0.093 | 0.257 |
| Clearance | 0.227 | 0.341 | 0.209 | 0.333 | 0.203 | 0.338 |
| | 0.979 | 0.971 | 0.971 | 0.967 | 0.792 | 0.852 |
| Free | 0.566 | 0.912 | 0.676 | 1.051 | 1.698 | 1.974 |
| Solvation | 0.902 | 1.409 | 1.057 | 1.487 | 2.719 | 3.061 |
| | 0.201 | 0.478 | 0.319 | 0.440 | 0.195 | 0.444 |
| Microsomal | 0.142 | 0.447 | 0.054 | 0.446 | 0.058 | 0.442 |
| Clearance | 0.232 | 0.558 | 0.195 | 0.556 | 0.201 | 0.549 |
| | 0.848 | 0.840 | 0.666 | 0.776 | 0.763 | 0.818 |
| Hemolytic | 0.326 | 0.494 | 0.379 | 0.580 | 0.320 | 0.513 |
| Toxicity | 0.482 | 0.645 | 0.657 | 0.741 | 0.570 | 0.673 |
| | 0.424 | 0.394 | 0.057 | 0.438 | 0.455 | 0.430 |
| Hepatic | 0.130 | 0.501 | 0.076 | 0.459 | 0.065 | 0.459 |
| Clearance | 0.223 | 0.627 | 0.237 | 0.571 | 0.202 | 0.571 |
| | 0.693 | 0.848 | 0.773 | 0.844 | 0.696 | 0.824 |
| Caco2 | 0.222 | 0.453 | 0.149 | 0.465 | 0.155 | 0.480 |
| | 0.339 | 0.583 | 0.290 | 0.588 | 0.329 | 0.622 |
| | 0.961 | 0.958 | 0.950 | 0.950 | 0.792 | 0.841 |
| Aqueous | 0.471 | 0.661 | 0.511 | 0.721 | 1.036 | 1.269 |
| Solubility | 0.644 | 0.871 | 0.730 | 0.954 | 1.421 | 1.656 |
| Volume of | 0.721 | 0.744 | 0.749 | 0.699 | 0.763 | 0.733 |
| Distribution | 0.230 | 0.473 | 0.174 | 0.507 | 0.170 | 0.485 |
| at Steady | 0.350 | 0.624 | 0.334 | 0.671 | 0.326 | 0.631 |
| State | 0.386 | 0.533 | 0.368 | 0.503 | 0.474 | 0.528 |
| Half-life | 0.361 | 0.598 | 0.281 | 0.609 | 0.264 | 0.590 |
| | 0.569 | 0.782 | 0.555 | 0.792 | 0.515 | 0.765 |

### Example 2

Active learning is a powerful concept in molecular machine learning that allows algorithms to guide iterative experiments to improve model performance and identify the most optimal molecular solutions. Many prominent studies have shown the potential for active learning to accelerate and de-risk the identification of optimal chemical reaction conditions and steer molecular optimization for drug discovery. Active learning is particularly powerful during early project stages. However, one major downside is that only a very small amount of training data is available to learn from which can be insufficient to support the accurate training of data-hungry machine learning models.

We previously showed that leveraging pairwise molecular representations as training data can support molecular optimization by directly training on and predicting property differences between molecules. Compared to classic molecular machine learning algorithms, which are trained to predict absolute property values, such paired approaches are more well-equipped to guide molecular optimization by directly learning from and predicting molecular property differences and by cancelling systematic assay errors. Beyond superior performance in anticipating property improvements between molecules, the molecular pairing approach shows particularly strong performance on very small datasets by benefiting from combinatorial data expansion through the pairing of molecules. Based on these findings, we hypothesized that we could implement exploitative active learning campaigns based on a molecular pairing approach (‘ActiveDelta’) to support rapid identification of the most potent inhibitors across a wide range of benchmark drug targets.

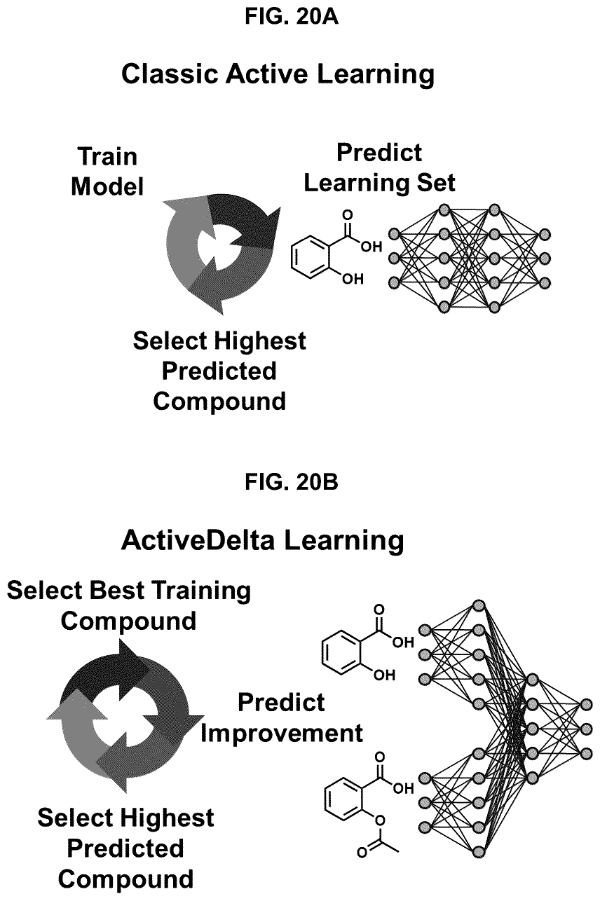

Classically during exploitative active learning, the machine learning model is trained on the available training data and the next compound to be added to the training dataset is selected based on which compound from the learning set has the highest predicted value (FIG. 20A). For ActiveDelta learning, training data is paired to learn property differences between molecules. Then, the next compound is selected based on which compound has the greatest predicted improvement from the most promising compound currently in the training dataset (FIG. 20B).

Described herein is the ActiveDelta concept and evaluate the Chemprop-based and XGBoost-based implementations of this learning strategy against standard exploitative active learning implementations of Chemprop, XGBoost, and Random Forest across 99 Ki datasets with simulated time splits. Across these benchmarks, the ActiveDelta approach quickly outcompeted standard active learning implementations, possibly by benefiting from the combinatorial expansion of data during pairing which enables the more accurate training of machine learning algorithms. The ActiveDelta implementations also enabled the discovery of more diverse molecules based on their Murcko scaffolds. Finally, the acquired data enabled the algorithms to predict the most promising compounds more accurately in time-split test datasets. Taken together, we believe that the ActiveDelta concept and extensions thereof hold large potential to further improve popular active learning campaigns by more directly training machine learning algorithms to guide molecular optimization and by combinatorically expanding small datasets to improve learning.

### Datasets

Datasets were obtained from Landrum et al., Cheminform 15:119 (2023) which utilized their simulated medicinal chemistry project data (SIMPD) algorithm to curate and split 99 ChEMBL Ki datasets with consistent values for target id, assay organism, assay category, and BioAssay Ontology (BAO) format into training and testing sets to simulate time-based splits. Duplicate molecules were removed. For initial active learning training dataset formation, two random datapoints were selected from each original training dataset and the remaining training datapoints were kept in the learning datasets. Exploitative active learning was repeated three times with unique starting datapoint pairs. Test sets were not used during active learning but were used in the test-set evaluation of all algorithms.

### Model Architecture and Implementation

To evaluate ActiveDelta with a deep machine learning model, we used the previously established, two-molecule version of the directed Message Passing Neural Network (D-MPNN) Chemprop. For our evaluation with tree-based models, we selected XGBoost with readily available GPU acceleration. Standard, single-molecule machine learning models were implemented using the single molecule-mode of Chemprop as well as XGBoost and Random Forest models as implemented in scikit-learn.

The Chemprop-based models were implemented for regression with num_folds=1, split_sizes=(1, 0, 0), ensemble_size=1, and aggregation=‘sum’ using the PyTorch deep learning framework. For the single-molecule Chemprop implementation, number_of_molecules=1 while for the ActiveDelta implementation number_of_molecules=2 to allow for processing of multiple inputs. We previously optimized the number of epochs for single and paired implementations of Chemprop and set epochs=5 for the ActiveDelta approach and epochs=50 for the single molecule active learning implementation of Chemprop. XGBoost and Random Forest regression machine learning models were implemented with default parameters and molecules were described using radial chemical fingerprints (Morgan Fingerprint, radius 2, 2048 bits, rdkit.org) when used as inputs for these models. For the ActiveDelta implementation of XGBoost, we still used default parameters and concatenated the fingerprints of each molecule in the molecular pairs were concatenated to create paired molecular representations.

During active learning, standard approaches were trained on the training set and used to predict the absolute value of each molecule in the learning dataset. The datapoint with highest predicted potency was then added to the training set for the next iteration of active learning (FIG. 20A). Conversely, during ActiveDelta learning, training was performed on the cross-merged training dataset to learn potency differences between molecular pairs. Then, the most potent molecule in the training set was paired with every molecule in the learning set to create new pairs for predictions on the learning data (FIG. 20B). The second molecule from the molecular pair with highest predicted potency improvement was added to the training set for the next iteration of active learning. For all active learning runs, analysis was repeated three times, each with a random pair of starting molecules for statistical analysis.

### Evaluation of Model Performance

To measure model performance during exploitative active learning, we analyzed model ability to correctly identify the top ten percentile of most potent compounds in the learning set. The non-parametric Wilcoxon signed-rank tests were performed for all statistical comparisons following three repeats of active learning. For plotting of chemical space, molecules were represented by radial chemical fingerprints (Morgan Fingerprint, radius 2, 2048 bits, rdkit.org). Principle Component Analysis (PCA) was first performed to reduce the 2048 input dimensions to 50 dimensions before t-distributed Stochastic Neighbor Embedding (t-SNE) was applied to further reduce these 50 dimensions to 2 dimensions. PCA and t-SNE were performed with scikit-learn and plotted with matplotlib. Bar plots were made in GraphPad Prism 10.2.0. Code and data for all these calculations can be found at github.com/RekerLab/ActiveDelta.

### Identifying the Most Potent Leads During Active Learning

First, we evaluated how directly learning from and predicting potency differences of molecular pairs affects adaptive learning by directly comparing the performance of specific machine learning algorithms when either applied to molecular pairs or in a classic single-molecule mode. Specifically, we evaluated the ability of the D-MPNN Chemprop and the gradient boosting tree model XGBoost to adaptively learn on molecular pairs using the ActiveDelta approach compared to their standard active learning implementations in single-molecule mode (FIG. 20A). As our measure of success, we analyzed all the model's ability to identify the most potent compounds (top ten percentile) during exploitative active learning. We cold-started active learning by selecting only two random datapoints as initial training data and allowed the models to iteratively select the next molecule from the learning set they predicted as the most potent compound to add to their training data. To improve readability, we refer to our predictive pipeline consisting of our molecular pair pre-processing approach and the established two-molecule version of Chemprop as “ActiveDelta Chemprop” (AD-CP) and the standard active learning implementation of single-molecule Chemprop as “Chemprop”. Similarly, we refer to our pairing approach applied to XGBoost as “ActiveDelta XGBoost” (AD-XGB) and the standard single-molecule active learning implementation of XGBoost as “XGBoost.”

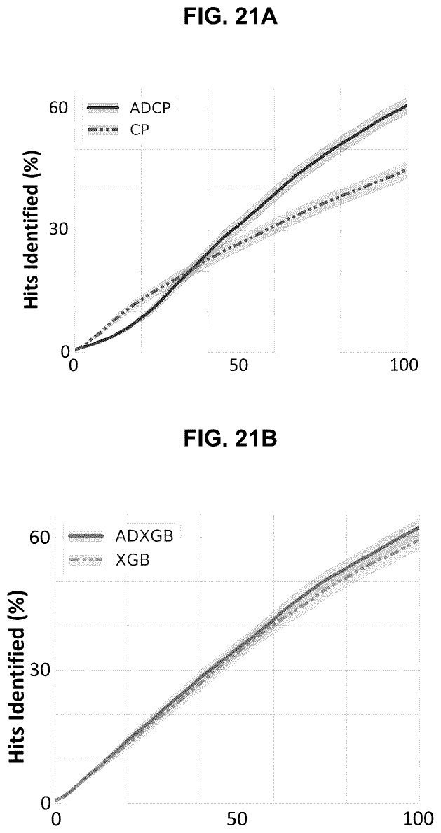

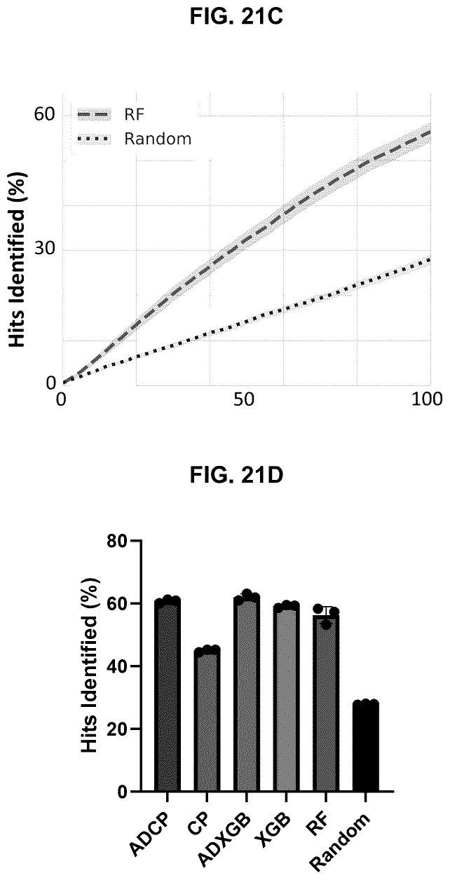

When comparing the deep machine learning implementations, we observed interesting patterns. AD-CP initially underperformed compared to the single-molecule implementation of Chemprop, potentially due to the increased complexity of learning and predicting potency improvements between molecular pairs compared to simply identifying analogs of the most promising compound identified so far. However, AD-CP quickly caught up and rapidly outcompeted the single-molecule active learning implementation of CP. We noted that AD-CP identified a statistically significantly larger fraction of the top ten percentile of most potent compounds compared to single-molecule CP after 100 iterations of active learning (45% vs. 61%, p=2×10⁻³³, FIG. 21A, Table 7). This improved performance extended out to 200 iterations where AD-CP had identified almost 90% of the most potent inhibitors (79% vs. 88%, p=4×10⁻¹⁹, Table 7). This data overall suggests that, while the learning from and predicting of molecular pairs might be more challenging with very limited data (<35 datapoints), the pairing rapidly enables combinatorial training data expansion that allows the more effective usage of deep neural networks for the identification of the most potent compounds from limited training data until almost all hits are selected.

A slightly different pattern emerged when comparing the tree-based implementations. AD-XGB and XGBoost initially selected similar numbers of the most potent molecules, potentially attesting to the more robust training of tree-based models on very small datasets. After 13 iterations, AD-XGB started consistently outperforming XGBoost. We noted that AD-XGB was selecting a larger fraction of the most potent molecules at 100 iterations (62% vs. 59%, p=0.001, FIG. 21 B, Table 7) and at 200 iterations (88% vs. 86%, p=0.02, Table 7). This further attests to the power of our pairing approach and shows that tree-based machine learning models can also benefit from the pairing to identify the most potent inhibitors in adaptive learning campaigns.

When comparing the performance of the tree-based and the deep neural network-based ActiveDelta approaches, we observed that AD-CP and AD-XGB showed no statistically significant difference at 100 iterations (p=0.2, FIG. 21A-B, Table 7) or 200 iterations (p=0.7, Table 7). This suggests that the improved performance of the active learning campaigns is largely driven by the pairing and can be implemented with various underlying, established machine learning algorithms.

We next evaluated how the paired approaches were performing overall compared to standard, single molecule active learning implementations. AD-XGB outcompeted all standard implementations at 100 iterations (p<0.001, FIG. 21A-D, Table 7) while AD-CP outcompeted all standard implementations at 100 iterations (p<0.002, FIG. 21A-D, Table 7) except for XGBoost over which it showed a nonsignificant improvement (p=0.3, FIG. 21A-D, Table 7). By 200 iterations, both models using the ActiveDelta approach selected more of the most potent leads than any standard single-molecule active learning approach (p<0.04, Table 7). These results highlight how a paired approach can allow models to rapidly learn in low data regimes to outcompete standard active learning implementations in identifying the most potent compounds. It also suggests that the Chemprop-based implementation requires more data than the tree-based implementation to outcompete standard approaches, potentially hinting at the larger data requirements for deep neural networks even when combinatorically expanding datasets through pairing.

TABLE 7
Percent of the top ten percentile most potent leads identified by random selection (Random),
Random Forest (RF), Chemprop (CP), AD-CP (ADCP), XGBoost (XGB), and AD-XGB (ADXGB) during
active learning across 99 Ki datasets for 200 iterations after starting from two random
datapoints. Average and standard error of the mean (SEM) shown for three replicates.

| Iterat. | Random | RF | CP | ADCP | XGB | ADXGB |
|---|---|---|---|---|---|---|
| 1 | 0.919 ± 0.165 | 0.982 ± 0.19 | 0.933 ± 0.162 | 0.922 ± 0.163 | 1.004 ± 0.175 | 1.03 ± 0.177 |
| 2 | 1.217 ± 0.197 | 1.529 ± 0.262 | 1.297 ± 0.22 | 1.135 ± 0.183 | 1.421 ± 0.217 | 1.283 ± 0.197 |
| 3 | 1.489 ± 0.218 | 2.053 ± 0.339 | 1.828 ± 0.28 | 1.408 ± 0.209 | 1.93 ± 0.273 | 1.882 ± 0.266 |
| 4 | 1.763 ± 0.23 | 2.585 ± 0.395 | 2.55 ± 0.357 | 1.63 ± 0.237 | 2.591 ± 0.335 | 2.41 ± 0.312 |
| 5 | 2.021 ± 0.251 | 3.13 ± 0.452 | 3.2 ± 0.423 | 1.868 ± 0.266 | 3.222 ± 0.397 | 3.17 ± 0.381 |
| 6 | 2.372 ± 0.267 | 3.823 ± 0.527 | 3.788 ± 0.48 | 2.125 ± 0.306 | 3.864 ± 0.453 | 3.914 ± 0.443 |
| 7 | 2.667 ± 0.28 | 4.423 ± 0.575 | 4.395 ± 0.544 | 2.501 ± 0.327 | 4.659 ± 0.521 | 4.63 ± 0.496 |
| 8 | 2.971 ± 0.289 | 5.123 ± 0.645 | 5.027 ± 0.613 | 2.804 ± 0.352 | 5.38 ± 0.575 | 5.377 ± 0.552 |
| 9 | 3.243 ± 0.316 | 5.766 ± 0.689 | 5.802 ± 0.682 | 3.1 ± 0.377 | 6.088 ± 0.644 | 6.093 ± 0.617 |
| 10 | 3.575 ± 0.328 | 6.463 ± 0.746 | 6.542 ± 0.762 | 3.372 ± 0.398 | 6.714 ± 0.69 | 6.832 ± 0.673 |
| 11 | 3.95 ± 0.354 | 7.155 ± 0.794 | 7.274 ± 0.837 | 3.753 ± 0.416 | 7.406 ± 0.741 | 7.382 ± 0.704 |
| 12 | 4.293 ± 0.371 | 7.816 ± 0.849 | 7.965 ± 0.895 | 4.169 ± 0.448 | 8.096 ± 0.77 | 8.046 ± 0.741 |
| 13 | 4.528 ± 0.381 | 8.581 ± 0.91 | 8.601 ± 0.954 | 4.593 ± 0.498 | 8.675 ± 0.796 | 8.777 ± 0.784 |
| 14 | 4.711 ± 0.383 | 9.319 ± 0.958 | 9.304 ± 1.005 | 5.07 ± 0.54 | 9.373 ± 0.83 | 9.581 ± 0.83 |
| 15 | 4.975 ± 0.394 | 9.976 ± 1.013 | 10.025 ± 1.058 | 5.507 ± 0.58 | 9.925 ± 0.865 | 10.287 ± 0.88 |
| 16 | 5.186 ± 0.401 | 10.58 ± 1.053 | 10.6 ± 1.093 | 5.993 ± 0.612 | 10.656 ± 0.901 | 10.996 ± 0.919 |
| 17 | 5.467 ± 0.419 | 11.363 ± 1.086 | 11.206 ± 1.125 | 6.554 ± 0.646 | 11.23 ± 0.946 | 11.795 ± 0.96 |
| 18 | 5.696 ± 0.427 | 12.034 ± 1.13 | 11.811 ± 1.175 | 7.165 ± 0.704 | 11.962 ± 0.983 | 12.525 ± 1.008 |
| 19 | 6.125 ± 0.446 | 12.776 ± 1.176 | 12.311 ± 1.212 | 7.81 ± 0.758 | 12.593 ± 1.031 | 13.305 ± 1.037 |
| 20 | 6.335 ± 0.456 | 13.431 ± 1.216 | 12.788 ± 1.236 | 8.379 ± 0.817 | 13.265 ± 1.072 | 14.158 ± 1.087 |
| 21 | 6.613 ± 0.46 | 14.226 ± 1.267 | 13.364 ± 1.268 | 9.046 ± 0.882 | 13.835 ± 1.113 | 14.879 ± 1.122 |
| 22 | 6.836 ± 0.472 | 14.877 ± 1.308 | 13.976 ± 1.298 | 9.67 ± 0.925 | 14.617 ± 1.157 | 15.614 ± 1.159 |
| 23 | 7.065 ± 0.481 | 15.486 ± 1.334 | 14.503 ± 1.328 | 10.403 ± 0.987 | 15.348 ± 1.186 | 16.231 ± 1.186 |
| 24 | 7.339 ± 0.488 | 16.179 ± 1.374 | 14.97 ± 1.343 | 11.067 ± 1.033 | 15.999 ± 1.224 | 16.895 ± 1.218 |
| 25 | 7.655 ± 0.508 | 16.884 ± 1.408 | 15.412 ± 1.368 | 11.836 ± 1.08 | 16.686 ± 1.256 | 17.595 ± 1.245 |
| 26 | 7.94 ± 0.522 | 17.532 ± 1.437 | 15.92 ± 1.398 | 12.661 ± 1.132 | 17.461 ± 1.294 | 18.152 ± 1.267 |
| 27 | 8.157 ± 0.53 | 18.184 ± 1.462 | 16.434 ± 1.43 | 13.59 ± 1.191 | 18.13 ± 1.322 | 18.894 ± 1.293 |
| 28 | 8.384 ± 0.537 | 18.873 ± 1.48 | 16.895 ± 1.452 | 14.398 ± 1.244 | 18.893 ± 1.353 | 19.72 ± 1.338 |
| 29 | 8.631 ± 0.553 | 19.627 ± 1.509 | 17.371 ± 1.475 | 15.327 ± 1.296 | 19.553 ± 1.382 | 20.398 ± 1.362 |
| 30 | 8.846 ± 0.559 | 20.285 ± 1.529 | 17.813 ± 1.5 | 16.199 ± 1.341 | 20.192 ± 1.412 | 21.186 ± 1.389 |
| 31 | 9.091 ± 0.561 | 20.915 ± 1.552 | 18.217 ± 1.526 | 17.002 ± 1.39 | 20.838 ± 1.447 | 21.893 ± 1.414 |
| 32 | 9.364 ± 0.562 | 21.542 ± 1.58 | 18.719 ± 1.548 | 17.822 ± 1.436 | 21.416 ± 1.474 | 22.581 ± 1.437 |
| 33 | 9.678 ± 0.575 | 22.11 ± 1.594 | 19.179 ± 1.586 | 18.597 ± 1.463 | 22.098 ± 1.502 | 23.337 ± 1.466 |
| 34 | 9.995 ± 0.582 | 22.737 ± 1.616 | 19.679 ± 1.6 | 19.426 ± 1.5 | 22.875 ± 1.534 | 23.91 ± 1.491 |
| 35 | 10.231 ± 0.585 | 23.326 ± 1.644 | 20.174 ± 1.618 | 20.257 ± 1.535 | 23.554 ± 1.553 | 24.588 ± 1.516 |
| 36 | 10.504 ± 0.599 | 24.019 ± 1.666 | 20.681 ± 1.649 | 21.017 ± 1.564 | 24.24 ± 1.568 | 25.326 ± 1.543 |
| 37 | 10.813 ± 0.613 | 24.601 ± 1.697 | 21.246 ± 1.68 | 21.858 ± 1.598 | 24.955 ± 1.593 | 26.033 ± 1.571 |
| 38 | 11.076 ± 0.631 | 25.133 ± 1.708 | 21.773 ± 1.701 | 22.706 ± 1.625 | 25.636 ± 1.609 | 26.705 ± 1.599 |
| 39 | 11.385 ± 0.643 | 25.689 ± 1.72 | 22.261 ± 1.711 | 23.532 ± 1.648 | 26.322 ± 1.627 | 27.392 ± 1.623 |
| 40 | 11.644 ± 0.645 | 26.374 ± 1.734 | 22.761 ± 1.721 | 24.342 ± 1.668 | 27.0 ± 1.65 | 28.332 ± 1.648 |
| 41 | 11.882 ± 0.659 | 27.016 ± 1.76 | 23.095 ± 1.732 | 25.169 ± 1.696 | 27.712 ± 1.672 | 28.881 ± 1.666 |
| 42 | 12.084 ± 0.668 | 27.637 ± 1.783 | 23.568 ± 1.751 | 26.014 ± 1.72 | 28.374 ± 1.687 | 29.494 ± 1.676 |
| 43 | 12.272 ± 0.673 | 28.226 ± 1.801 | 24.044 ± 1.765 | 26.785 ± 1.741 | 29.153 ± 1.712 | 30.165 ± 1.687 |
| 44 | 12.528 ± 0.684 | 28.897 ± 1.814 | 24.568 ± 1.794 | 27.622 ± 1.771 | 29.874 ± 1.732 | 30.781 ± 1.706 |
| 45 | 12.74 ± 0.688 | 29.404 ± 1.831 | 24.931 ± 1.809 | 28.432 ± 1.796 | 30.534 ± 1.758 | 31.462 ± 1.727 |
| 46 | 13.018 ± 0.695 | 29.881 ± 1.837 | 25.372 ± 1.827 | 29.035 ± 1.814 | 31.31 ± 1.786 | 32.029 ± 1.738 |
| 47 | 13.339 ± 0.712 | 30.561 ± 1.852 | 25.758 ± 1.832 | 29.741 ± 1.825 | 31.917 ± 1.792 | 32.653 ± 1.755 |
| 48 | 13.637 ± 0.717 | 31.157 ± 1.874 | 26.118 ± 1.847 | 30.395 ± 1.84 | 32.512 ± 1.797 | 33.363 ± 1.779 |
| 49 | 13.877 ± 0.723 | 31.793 ± 1.886 | 26.472 ± 1.847 | 30.989 ± 1.852 | 33.082 ± 1.809 | 34.121 ± 1.819 |
| 50 | 14.161 ± 0.73 | 32.399 ± 1.902 | 26.93 ± 1.855 | 31.669 ± 1.864 | 33.821 ± 1.825 | 34.739 ± 1.831 |
| 51 | 14.497 ± 0.735 | 32.904 ± 1.916 | 27.194 ± 1.858 | 32.366 ± 1.895 | 34.554 ± 1.843 | 35.363 ± 1.853 |
| 52 | 14.786 ± 0.752 | 33.471 ± 1.934 | 27.658 ± 1.862 | 33.124 ± 1.913 | 35.167 ± 1.852 | 35.922 ± 1.872 |
| 53 | 15.145 ± 0.769 | 33.977 ± 1.945 | 28.108 ± 1.858 | 33.895 ± 1.931 | 35.795 ± 1.869 | 36.566 ± 1.896 |
| 54 | 15.468 ± 0.776 | 34.531 ± 1.959 | 28.521 ± 1.864 | 34.565 ± 1.948 | 36.475 ± 1.878 | 37.149 ± 1.91 |
| 55 | 15.662 ± 0.779 | 35.025 ± 1.963 | 28.953 ± 1.869 | 35.126 ± 1.951 | 37.118 ± 1.902 | 37.78 ± 1.929 |
| 56 | 15.925 ± 0.79 | 35.631 ± 1.976 | 29.361 ± 1.87 | 35.897 ± 1.968 | 37.784 ± 1.918 | 38.549 ± 1.941 |
| 57 | 16.151 ± 0.792 | 36.187 ± 1.981 | 29.789 ± 1.88 | 36.698 ± 1.968 | 38.406 ± 1.926 | 39.226 ± 1.948 |
| 58 | 16.372 ± 0.806 | 36.862 ± 1.99 | 30.251 ± 1.886 | 37.456 ± 1.986 | 38.934 ± 1.935 | 39.904 ± 1.962 |
| 59 | 16.583 ± 0.807 | 37.512 ± 2.009 | 30.658 ± 1.889 | 38.05 ± 1.989 | 39.603 ± 1.952 | 40.543 ± 1.977 |
| 60 | 16.792 ± 0.802 | 37.991 ± 2.017 | 31.024 ± 1.891 | 38.821 ± 2.003 | 40.241 ± 1.976 | 41.291 ± 1.998 |
| 61 | 17.077 ± 0.803 | 38.513 ± 2.026 | 31.42 ± 1.905 | 39.593 ± 2.015 | 40.759 ± 1.996 | 42.067 ± 2.023 |
| 62 | 17.381 ± 0.808 | 39.085 ± 2.044 | 31.776 ± 1.905 | 40.23 ± 2.018 | 41.366 ± 2.012 | 42.823 ± 2.033 |
| 63 | 17.657 ± 0.817 | 39.736 ± 2.056 | 32.271 ± 1.91 | 41.053 ± 2.025 | 41.821 ± 2.026 | 43.48 ± 2.041 |
| 64 | 17.952 ± 0.825 | 40.311 ± 2.073 | 32.703 ± 1.911 | 41.708 ± 2.042 | 42.376 ± 2.035 | 44.101 ± 2.04 |
| 65 | 18.131 ± 0.82 | 40.831 ± 2.074 | 33.058 ± 1.921 | 42.364 ± 2.049 | 42.814 ± 2.041 | 44.783 ± 2.065 |
| 66 | 18.299 ± 0.831 | 41.368 ± 2.083 | 33.404 ± 1.929 | 43.142 ± 2.056 | 43.466 ± 2.044 | 45.373 ± 2.077 |
| 67 | 18.504 ± 0.834 | 41.977 ± 2.097 | 33.72 ± 1.931 | 43.861 ± 2.075 | 43.973 ± 2.04 | 45.985 ± 2.081 |
| 68 | 18.865 ± 0.843 | 42.455 ± 2.11 | 34.113 ± 1.932 | 44.361 ± 2.09 | 44.467 ± 2.046 | 46.592 ± 2.089 |
| 69 | 19.107 ± 0.847 | 42.936 ± 2.115 | 34.497 ± 1.945 | 44.936 ± 2.092 | 45.002 ± 2.054 | 47.147 ± 2.094 |
| 70 | 19.324 ± 0.847 | 43.468 ± 2.127 | 34.857 ± 1.964 | 45.626 ± 2.107 | 45.512 ± 2.062 | 47.761 ± 2.102 |
| 71 | 19.597 ± 0.857 | 43.92 ± 2.13 | 35.271 ± 1.968 | 46.179 ± 2.115 | 46.082 ± 2.071 | 48.348 ± 2.104 |
| 72 | 19.832 ± 0.871 | 44.413 ± 2.144 | 35.622 ± 1.979 | 46.723 ± 2.111 | 46.752 ± 2.085 | 48.938 ± 2.104 |
| 73 | 20.138 ± 0.881 | 44.955 ± 2.16 | 35.902 ± 1.977 | 47.293 ± 2.108 | 47.348 ± 2.093 | 49.497 ± 2.105 |
| 74 | 20.41 ± 0.888 | 45.504 ± 2.176 | 36.314 ± 1.996 | 47.858 ± 2.106 | 47.99 ± 2.11 | 50.087 ± 2.109 |
| 75 | 20.692 ± 0.902 | 45.991 ± 2.181 | 36.709 ± 2.003 | 48.537 ± 2.12 | 48.481 ± 2.128 | 50.598 ± 2.106 |
| 76 | 21.09 ± 0.92 | 46.437 ± 2.185 | 37.061 ± 2.006 | 49.038 ± 2.111 | 48.962 ± 2.135 | 51.029 ± 2.1 |
| 77 | 21.364 ± 0.926 | 46.829 ± 2.191 | 37.404 ± 2.012 | 49.606 ± 2.105 | 49.441 ± 2.154 | 51.522 ± 2.1 |
| 78 | 21.645 ± 0.92 | 47.323 ± 2.192 | 37.725 ± 2.025 | 50.206 ± 2.104 | 49.916 ± 2.164 | 51.897 ± 2.105 |
| 79 | 21.977 ± 0.924 | 47.734 ± 2.192 | 38.002 ± 2.033 | 50.723 ± 2.113 | 50.308 ± 2.168 | 52.346 ± 2.106 |
| 80 | 22.299 ± 0.936 | 48.254 ± 2.199 | 38.354 ± 2.04 | 51.216 ± 2.111 | 50.773 ± 2.182 | 52.863 ± 2.112 |
| 81 | 22.521 ± 0.94 | 48.742 ± 2.201 | 38.735 ± 2.055 | 51.738 ± 2.108 | 51.198 ± 2.183 | 53.394 ± 2.119 |
| 82 | 22.803 ± 0.964 | 49.237 ± 2.212 | 39.04 ± 2.066 | 52.196 ± 2.1 | 51.754 ± 2.184 | 53.905 ± 2.121 |
| 83 | 23.067 ± 0.984 | 49.678 ± 2.224 | 39.333 ± 2.071 | 52.68 ± 2.097 | 52.239 ± 2.19 | 54.32 ± 2.117 |
| 84 | 23.343 ± 0.991 | 50.151 ± 2.22 | 39.699 ± 2.086 | 53.131 ± 2.096 | 52.658 ± 2.189 | 54.814 ± 2.111 |
| 85 | 23.673 ± 1.011 | 50.491 ± 2.222 | 39.943 ± 2.089 | 53.641 ± 2.097 | 53.061 ± 2.191 | 55.226 ± 2.112 |
| 86 | 23.874 ± 1.017 | 50.88 ± 2.22 | 40.356 ± 2.101 | 54.244 ± 2.108 | 53.596 ± 2.192 | 55.602 ± 2.115 |
| 87 | 24.216 ± 1.034 | 51.267 ± 2.218 | 40.646 ± 2.114 | 54.752 ± 2.109 | 53.988 ± 2.193 | 56.08 ± 2.121 |
| 88 | 24.474 ± 1.039 | 51.601 ± 2.22 | 40.972 ± 2.121 | 55.187 ± 2.104 | 54.45 ± 2.195 | 56.557 ± 2.114 |
| 89 | 24.753 ± 1.045 | 51.962 ± 2.21 | 41.339 ± 2.135 | 55.731 ± 2.097 | 54.858 ± 2.201 | 57.081 ± 2.111 |
| 90 | 25.014 ± 1.047 | 52.349 ± 2.214 | 41.611 ± 2.135 | 56.322 ± 2.1 | 55.207 ± 2.2 | 57.577 ± 2.107 |
| 91 | 25.36 ± 1.061 | 52.758 ± 2.203 | 41.935 ± 2.14 | 56.799 ± 2.089 | 55.499 ± 2.202 | 58.011 ± 2.098 |
| 92 | 25.597 ± 1.063 | 53.259 ± 2.199 | 42.206 ± 2.149 | 57.241 ± 2.081 | 55.91 ± 2.194 | 58.521 ± 2.092 |
| 93 | 25.782 ± 1.064 | 53.64 ± 2.195 | 42.582 ± 2.152 | 57.788 ± 2.075 | 56.243 ± 2.193 | 58.952 ± 2.086 |
| 94 | 26.134 ± 1.073 | 54.042 ± 2.199 | 42.887 ± 2.159 | 58.217 ± 2.066 | 56.661 ± 2.179 | 59.484 ± 2.08 |
| 95 | 26.394 ± 1.078 | 54.372 ± 2.201 | 43.219 ± 2.162 | 58.588 ± 2.062 | 57.19 ± 2.175 | 59.905 ± 2.071 |
| 96 | 26.663 ± 1.088 | 54.725 ± 2.203 | 43.501 ± 2.165 | 59.044 ± 2.06 | 57.535 ± 2.169 | 60.353 ± 2.064 |
| 97 | 27.007 ± 1.103 | 55.133 ± 2.204 | 43.875 ± 2.184 | 59.467 ± 2.057 | 58.009 ± 2.168 | 60.773 ± 2.064 |
| 98 | 27.381 ± 1.127 | 55.594 ± 2.207 | 44.098 ± 2.189 | 59.841 ± 2.049 | 58.43 ± 2.167 | 61.16 ± 2.052 |
| 99 | 27.629 ± 1.131 | 55.986 ± 2.208 | 44.662 ± 2.202 | 60.342 ± 2.043 | 58.803 ± 2.151 | 61.621 ± 2.04 |
| 100 | 27.939 ± 1.141 | 56.329 ± 2.205 | 45.031 ± 2.207 | 60.812 ± 2.041 | 59.185 ± 2.147 | 62.055 ± 2.037 |
| 101 | 28.168 ± 1.143 | 56.648 ± 2.204 | 45.475 ± 2.202 | 61.307 ± 2.037 | 59.492 ± 2.144 | 62.494 ± 2.035 |
| 102 | 28.475 ± 1.144 | 57.06 ± 2.204 | 45.905 ± 2.199 | 61.776 ± 2.036 | 59.895 ± 2.136 | 62.885 ± 2.024 |
| 103 | 28.738 ± 1.144 | 57.438 ± 2.213 | 46.474 ± 2.211 | 62.171 ± 2.029 | 60.258 ± 2.133 | 63.261 ± 2.015 |
| 104 | 29.052 ± 1.16 | 57.791 ± 2.222 | 47.063 ± 2.224 | 62.542 ± 2.02 | 60.65 ± 2.133 | 63.681 ± 2.001 |
| 105 | 29.295 ± 1.173 | 58.106 ± 2.228 | 47.518 ± 2.228 | 63.007 ± 2.017 | 61.087 ± 2.131 | 64.088 ± 1.997 |
| 106 | 29.494 ± 1.178 | 58.47 ± 2.239 | 48.043 ± 2.235 | 63.471 ± 2.007 | 61.487 ± 2.133 | 64.461 ± 1.998 |
| 107 | 29.655 ± 1.179 | 58.904 ± 2.241 | 48.509 ± 2.227 | 63.858 ± 2.003 | 61.801 ± 2.121 | 64.866 ± 1.994 |
| 108 | 29.875 ± 1.183 | 59.182 ± 2.237 | 48.892 ± 2.219 | 64.359 ± 1.989 | 62.173 ± 2.115 | 65.233 ± 1.993 |
| 109 | 30.128 ± 1.183 | 59.56 ± 2.243 | 49.257 ± 2.227 | 64.807 ± 1.975 | 62.573 ± 2.108 | 65.539 ± 1.992 |
| 110 | 30.478 ± 1.193 | 59.838 ± 2.241 | 49.657 ± 2.232 | 65.172 ± 1.964 | 62.926 ± 2.099 | 65.895 ± 1.998 |
| 111 | 30.734 ± 1.187 | 60.162 ± 2.236 | 50.007 ± 2.234 | 65.468 ± 1.947 | 63.332 ± 2.099 | 66.148 ± 1.994 |
| 112 | 31.035 ± 1.193 | 60.539 ± 2.232 | 50.422 ± 2.235 | 65.909 ± 1.942 | 63.652 ± 2.084 | 66.492 ± 1.98 |
| 113 | 31.265 ± 1.201 | 60.879 ± 2.228 | 50.83 ± 2.235 | 66.39 ± 1.942 | 63.941 ± 2.077 | 66.796 ± 1.978 |
| 114 | 31.541 ± 1.21 | 61.207 ± 2.227 | 51.333 ± 2.248 | 66.845 ± 1.935 | 64.35 ± 2.074 | 67.126 ± 1.972 |
| 115 | 31.876 ± 1.214 | 61.54 ± 2.225 | 51.86 ± 2.247 | 67.286 ± 1.921 | 64.668 ± 2.069 | 67.444 ± 1.971 |
| 116 | 32.212 ± 1.222 | 61.915 ± 2.232 | 52.219 ± 2.254 | 67.726 ± 1.91 | 65.031 ± 2.062 | 67.741 ± 1.962 |
| 117 | 32.464 ± 1.236 | 62.331 ± 2.228 | 52.682 ± 2.252 | 68.151 ± 1.911 | 65.397 ± 2.054 | 68.068 ± 1.961 |
| 118 | 32.659 ± 1.243 | 62.66 ± 2.228 | 53.082 ± 2.252 | 68.464 ± 1.903 | 65.67 ± 2.048 | 68.38 ± 1.955 |
| 119 | 32.964 ± 1.257 | 62.978 ± 2.23 | 53.477 ± 2.261 | 68.786 ± 1.897 | 66.008 ± 2.039 | 68.682 ± 1.949 |
| 120 | 33.214 ± 1.259 | 63.405 ± 2.237 | 53.814 ± 2.253 | 69.093 ± 1.884 | 66.354 ± 2.037 | 69.105 ± 1.954 |
| 121 | 33.48 ± 1.262 | 63.71 ± 2.238 | 54.224 ± 2.244 | 69.34 ± 1.879 | 66.77 ± 2.032 | 69.422 ± 1.95 |
| 122 | 33.72 ± 1.272 | 64.115 ± 2.233 | 54.708 ± 2.244 | 69.588 ± 1.875 | 67.032 ± 2.024 | 69.756 ± 1.941 |
| 123 | 33.986 ± 1.277 | 64.402 ± 2.228 | 55.02 ± 2.25 | 69.901 ± 1.867 | 67.308 ± 2.015 | 70.085 ± 1.939 |
| 124 | 34.235 ± 1.276 | 64.777 ± 2.234 | 55.355 ± 2.261 | 70.144 ± 1.857 | 67.668 ± 2.004 | 70.515 ± 1.938 |
| 125 | 34.49 ± 1.276 | 65.202 ± 2.234 | 55.813 ± 2.264 | 70.384 ± 1.841 | 68.023 ± 1.995 | 70.913 ± 1.935 |
| 126 | 34.78 ± 1.283 | 65.606 ± 2.234 | 56.188 ± 2.266 | 70.718 ± 1.827 | 68.335 ± 1.985 | 71.298 ± 1.932 |
| 127 | 35.072 ± 1.291 | 65.993 ± 2.236 | 56.499 ± 2.261 | 71.035 ± 1.812 | 68.661 ± 1.977 | 71.671 ± 1.924 |
| 128 | 35.254 ± 1.286 | 66.399 ± 2.23 | 56.901 ± 2.259 | 71.369 ± 1.807 | 69.047 ± 1.965 | 71.971 ± 1.919 |
| 129 | 35.621 ± 1.296 | 66.881 ± 2.225 | 57.223 ± 2.263 | 71.715 ± 1.788 | 69.402 ± 1.961 | 72.381 ± 1.912 |
| 130 | 35.855 ± 1.304 | 67.311 ± 2.22 | 57.54 ± 2.256 | 72.113 ± 1.771 | 69.821 ± 1.955 | 72.722 ± 1.907 |
| 131 | 36.192 ± 1.315 | 67.601 ± 2.219 | 57.868 ± 2.259 | 72.442 ± 1.757 | 70.052 ± 1.944 | 73.101 ± 1.904 |
| 132 | 36.41 ± 1.32 | 68.003 ± 2.214 | 58.193 ± 2.252 | 72.781 ± 1.746 | 70.494 ± 1.938 | 73.436 ± 1.897 |
| 133 | 36.688 ± 1.335 | 68.329 ± 2.214 | 58.556 ± 2.256 | 73.033 ± 1.733 | 70.897 ± 1.939 | 73.838 ± 1.888 |
| 134 | 36.943 ± 1.334 | 68.662 ± 2.209 | 59.006 ± 2.263 | 73.375 ± 1.721 | 71.25 ± 1.933 | 74.197 ± 1.882 |
| 135 | 37.2 ± 1.345 | 69.026 ± 2.207 | 59.356 ± 2.256 | 73.675 ± 1.71 | 71.551 ± 1.928 | 74.513 ± 1.877 |
| 136 | 37.48 ± 1.354 | 69.322 ± 2.204 | 59.684 ± 2.248 | 74.104 ± 1.7 | 71.822 ± 1.922 | 74.79 ± 1.871 |
| 137 | 37.756 ± 1.364 | 69.687 ± 2.202 | 60.09 ± 2.249 | 74.397 ± 1.687 | 72.11 ± 1.921 | 75.062 ± 1.872 |
| 138 | 37.934 ± 1.364 | 69.974 ± 2.202 | 60.453 ± 2.24 | 74.695 ± 1.673 | 72.449 ± 1.92 | 75.348 ± 1.867 |
| 139 | 38.184 ± 1.369 | 70.292 ± 2.195 | 60.789 ± 2.253 | 74.954 ± 1.661 | 72.743 ± 1.92 | 75.647 ± 1.859 |
| 140 | 38.496 ± 1.376 | 70.638 ± 2.186 | 61.141 ± 2.255 | 75.256 ± 1.653 | 73.053 ± 1.916 | 75.987 ± 1.85 |
| 141 | 38.768 ± 1.387 | 70.923 ± 2.184 | 61.453 ± 2.255 | 75.515 ± 1.647 | 73.389 ± 1.912 | 76.197 ± 1.844 |
| 142 | 39.122 ± 1.404 | 71.163 ± 2.176 | 61.747 ± 2.26 | 75.775 ± 1.645 | 73.621 ± 1.9 | 76.452 ± 1.839 |
| 143 | 39.381 ± 1.411 | 71.424 ± 2.174 | 62.101 ± 2.256 | 76.085 ± 1.641 | 73.904 ± 1.892 | 76.739 ± 1.83 |
| 144 | 39.623 ± 1.417 | 71.682 ± 2.166 | 62.396 ± 2.255 | 76.385 ± 1.636 | 74.139 ± 1.884 | 77.095 ± 1.823 |
| 145 | 39.832 ± 1.42 | 71.914 ± 2.163 | 62.766 ± 2.257 | 76.689 ± 1.63 | 74.406 ± 1.882 | 77.399 ± 1.81 |
| 146 | 40.126 ± 1.433 | 72.169 ± 2.159 | 62.994 ± 2.261 | 76.992 ± 1.625 | 74.632 ± 1.88 | 77.666 ± 1.802 |
| 147 | 40.419 ± 1.442 | 72.536 ± 2.153 | 63.315 ± 2.266 | 77.261 ± 1.624 | 74.928 ± 1.875 | 77.932 ± 1.793 |
| 148 | 40.678 ± 1.453 | 72.813 ± 2.152 | 63.625 ± 2.265 | 77.519 ± 1.617 | 75.221 ± 1.871 | 78.184 ± 1.787 |
| 149 | 40.952 ± 1.465 | 72.985 ± 2.144 | 64.026 ± 2.267 | 77.775 ± 1.611 | 75.506 ± 1.87 | 78.484 ± 1.779 |
| 150 | 41.258 ± 1.474 | 73.35 ± 2.137 | 64.395 ± 2.268 | 78.054 ± 1.614 | 75.732 ± 1.868 | 78.746 ± 1.778 |
| 151 | 41.6 ± 1.485 | 73.627 ± 2.124 | 64.766 ± 2.266 | 78.272 ± 1.606 | 75.93 ± 1.859 | 78.956 ± 1.771 |
| 152 | 41.887 ± 1.497 | 73.951 ± 2.115 | 65.1 ± 2.267 | 78.531 ± 1.601 | 76.163 ± 1.853 | 79.133 ± 1.761 |
| 153 | 42.151 ± 1.498 | 74.283 ± 2.103 | 65.494 ± 2.26 | 78.81 ± 1.6 | 76.384 ± 1.853 | 79.503 ± 1.759 |
| 154 | 42.347 ± 1.502 | 74.567 ± 2.099 | 65.838 ± 2.261 | 79.045 ± 1.597 | 76.627 ± 1.851 | 79.788 ± 1.752 |
| 155 | 42.629 ± 1.512 | 74.879 ± 2.092 | 66.324 ± 2.257 | 79.238 ± 1.593 | 76.83 ± 1.847 | 80.014 ± 1.746 |
| 156 | 42.842 ± 1.522 | 75.092 ± 2.085 | 66.821 ± 2.249 | 79.505 ± 1.584 | 77.083 ± 1.848 | 80.182 ± 1.74 |
| 157 | 43.093 ± 1.531 | 75.405 ± 2.081 | 67.248 ± 2.252 | 79.721 ± 1.575 | 77.397 ± 1.846 | 80.402 ± 1.736 |
| 158 | 43.298 ± 1.536 | 75.677 ± 2.077 | 67.61 ± 2.246 | 80.074 ± 1.572 | 77.647 ± 1.841 | 80.701 ± 1.728 |
| 159 | 43.577 ± 1.547 | 75.978 ± 2.075 | 68.006 ± 2.245 | 80.381 ± 1.567 | 78.008 ± 1.841 | 80.963 ± 1.72 |
| 160 | 43.759 ± 1.547 | 76.275 ± 2.07 | 68.381 ± 2.238 | 80.635 ± 1.563 | 78.321 ± 1.839 | 81.199 ± 1.709 |
| 161 | 44.126 ± 1.564 | 76.536 ± 2.067 | 68.792 ± 2.238 | 80.778 ± 1.558 | 78.64 ± 1.836 | 81.467 ± 1.699 |
| 162 | 44.339 ± 1.573 | 76.79 ± 2.066 | 69.1 ± 2.234 | 81.005 ± 1.55 | 78.879 ± 1.83 | 81.668 ± 1.689 |
| 163 | 44.618 ± 1.585 | 77.064 ± 2.058 | 69.46 ± 2.232 | 81.198 ± 1.542 | 79.09 ± 1.827 | 81.921 ± 1.682 |
| 164 | 44.861 ± 1.58 | 77.302 ± 2.052 | 69.848 ± 2.229 | 81.463 ± 1.542 | 79.341 ± 1.824 | 82.23 ± 1.674 |
| 165 | 45.117 ± 1.592 | 77.604 ± 2.049 | 70.223 ± 2.228 | 81.689 ± 1.534 | 79.524 ± 1.818 | 82.486 ± 1.668 |
| 166 | 45.34 ± 1.594 | 77.927 ± 2.047 | 70.517 ± 2.233 | 81.872 ± 1.529 | 79.761 ± 1.812 | 82.687 ± 1.662 |
| 167 | 45.637 ± 1.602 | 78.147 ± 2.045 | 70.846 ± 2.235 | 82.105 ± 1.519 | 79.975 ± 1.81 | 82.869 ± 1.659 |
| 168 | 45.839 ± 1.606 | 78.367 ± 2.034 | 71.101 ± 2.228 | 82.266 ± 1.514 | 80.232 ± 1.803 | 83.122 ± 1.648 |
| 169 | 46.13 ± 1.615 | 78.535 ± 2.03 | 71.388 ± 2.226 | 82.422 ± 1.509 | 80.403 ± 1.793 | 83.311 ± 1.643 |
| 170 | 46.392 ± 1.62 | 78.714 ± 2.02 | 71.616 ± 2.217 | 82.67 ± 1.504 | 80.628 ± 1.78 | 83.464 ± 1.634 |
| 171 | 46.758 ± 1.627 | 78.973 ± 2.013 | 71.917 ± 2.217 | 82.903 ± 1.501 | 80.851 ± 1.776 | 83.674 ± 1.628 |
| 172 | 47.062 ± 1.633 | 79.263 ± 2.009 | 72.212 ± 2.219 | 83.144 ± 1.493 | 81.022 ± 1.77 | 83.865 ± 1.624 |
| 173 | 47.274 ± 1.635 | 79.462 ± 2.003 | 72.474 ± 2.21 | 83.341 ± 1.49 | 81.284 ± 1.761 | 84.062 ± 1.614 |
| 174 | 47.588 ± 1.641 | 79.672 ± 1.992 | 72.805 ± 2.207 | 83.516 ± 1.483 | 81.497 ± 1.753 | 84.196 ± 1.613 |
| 175 | 47.877 ± 1.649 | 79.824 ± 1.99 | 73.123 ± 2.211 | 83.746 ± 1.474 | 81.733 ± 1.742 | 84.406 ± 1.605 |
| 176 | 48.177 ± 1.654 | 80.068 ± 1.987 | 73.393 ± 2.201 | 84.048 ± 1.469 | 81.916 ± 1.738 | 84.56 ± 1.599 |
| 177 | 48.479 ± 1.663 | 80.277 ± 1.979 | 73.678 ± 2.189 | 84.261 ± 1.467 | 82.193 ± 1.731 | 84.724 ± 1.594 |
| 178 | 48.766 ± 1.674 | 80.423 ± 1.972 | 73.995 ± 2.185 | 84.484 ± 1.455 | 82.38 ± 1.728 | 84.838 ± 1.589 |
| 179 | 49.032 ± 1.676 | 80.624 ± 1.967 | 74.241 ± 2.188 | 84.665 ± 1.447 | 82.569 ± 1.715 | 85.016 ± 1.585 |
| 180 | 49.264 ± 1.685 | 80.841 ± 1.96 | 74.524 ± 2.19 | 84.932 ± 1.452 | 82.755 ± 1.713 | 85.133 ± 1.576 |
| 181 | 49.422 ± 1.684 | 81.042 ± 1.956 | 74.79 ± 2.192 | 85.088 ± 1.447 | 82.977 ± 1.707 | 85.286 ± 1.563 |
| 182 | 49.623 ± 1.694 | 81.266 ± 1.948 | 75.006 ± 2.19 | 85.259 ± 1.439 | 83.157 ± 1.701 | 85.419 ± 1.555 |
| 183 | 49.963 ± 1.703 | 81.569 ± 1.95 | 75.293 ± 2.179 | 85.446 ± 1.429 | 83.461 ± 1.697 | 85.663 ± 1.548 |
| 184 | 50.159 ± 1.707 | 81.792 ± 1.944 | 75.521 ± 2.167 | 85.585 ± 1.425 | 83.614 ± 1.69 | 85.834 ± 1.543 |
| 185 | 50.507 ± 1.712 | 81.978 ± 1.941 | 75.785 ± 2.159 | 85.74 ± 1.421 | 83.801 ± 1.686 | 86.001 ± 1.542 |
| 186 | 50.765 ± 1.728 | 82.178 ± 1.94 | 76.032 ± 2.155 | 85.859 ± 1.421 | 84.059 ± 1.68 | 86.163 ± 1.537 |
| 187 | 51.009 ± 1.733 | 82.438 ± 1.942 | 76.287 ± 2.147 | 85.968 ± 1.418 | 84.301 ± 1.672 | 86.313 ± 1.534 |
| 188 | 51.263 ± 1.735 | 82.62 ± 1.933 | 76.58 ± 2.141 | 86.081 ± 1.41 | 84.509 ± 1.665 | 86.425 ± 1.531 |
| 189 | 51.608 ± 1.749 | 82.837 ± 1.923 | 76.845 ± 2.136 | 86.231 ± 1.408 | 84.704 ± 1.654 | 86.537 ± 1.52 |
| 190 | 51.868 ± 1.749 | 83.005 ± 1.918 | 77.115 ± 2.134 | 86.347 ± 1.406 | 84.853 ± 1.648 | 86.702 ± 1.516 |
| 191 | 52.143 ± 1.752 | 83.152 ± 1.916 | 77.393 ± 2.131 | 86.489 ± 1.399 | 84.977 ± 1.644 | 86.851 ± 1.51 |
| 192 | 52.479 ± 1.763 | 83.352 ± 1.903 | 77.602 ± 2.128 | 86.624 ± 1.397 | 85.137 ± 1.638 | 87.013 ± 1.497 |
| 193 | 52.681 ± 1.763 | 83.493 ± 1.893 | 77.807 ± 2.123 | 86.78 ± 1.39 | 85.312 ± 1.623 | 87.144 ± 1.49 |
| 194 | 52.969 ± 1.77 | 83.663 ± 1.887 | 77.964 ± 2.118 | 86.909 ± 1.387 | 85.464 ± 1.614 | 87.308 ± 1.483 |
| 195 | 53.323 ± 1.784 | 83.791 ± 1.879 | 78.196 ± 2.108 | 87.062 ± 1.383 | 85.57 ± 1.605 | 87.492 ± 1.476 |
| 196 | 53.589 ± 1.792 | 83.984 ± 1.868 | 78.429 ± 2.098 | 87.174 ± 1.376 | 85.707 ± 1.592 | 87.663 ± 1.466 |
| 197 | 53.825 ± 1.796 | 84.111 ± 1.861 | 78.607 ± 2.089 | 87.32 ± 1.368 | 85.841 ± 1.588 | 87.85 ± 1.459 |
| 198 | 54.096 ± 1.806 | 84.319 ± 1.855 | 78.818 ± 2.08 | 87.534 ± 1.364 | 85.978 ± 1.585 | 87.988 ± 1.447 |
| 199 | 54.362 ± 1.81 | 84.497 ± 1.85 | 79.067 ± 2.074 | 87.69 ± 1.359 | 86.221 ± 1.582 | 88.172 ± 1.44 |
| 200 | 54.532 ± 1.811 | 84.622 ± 1.839 | 79.273 ± 2.072 | 87.881 ± 1.356 | 86.347 ± 1.575 | 88.38 ± 1.434 |

### Chemical Diversity in Molecular Selection

Beyond their ability to identify the most potent inhibitors, we sought to determine how these approaches sampled chemical space. When analyzing the scaffold diversity of hits (i.e., the number of unique Murcko scaffolds in the set of molecules selected by the different approaches whose Ki values are within the top ten percentile of the most potent compounds in the complete learning set), AD-CP selected more distinct scaffolds than Chemprop (p=5×10⁻²⁵ at 100 iterations) but AD-XGB's increase in distinct scaffolds selected was not statistically significantly compared to XGBoost (p=0.1 at 100 iterations). Considering all approaches, AD-CP selected the largest number of distinct scaffolds in hits by 100 iterations (14.0±5.6 scaffolds on average) followed by AD-XGB (13.8±5.4), XGBoost (13.4±5.9), Random Forest (12.5±6.1), Chemprop (10.9±5.2), and then random selection (8.1±2.4). AD-CP, AD-XGB, and XGBoost showed no statistically significant differences, but all three approaches outperformed all other approaches at 100 iterations.

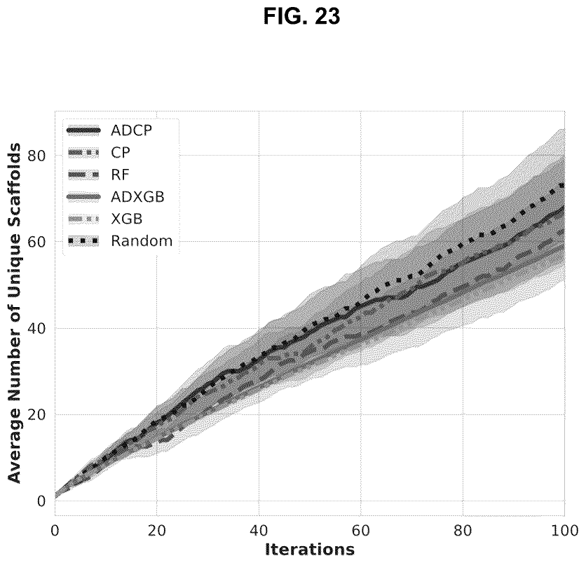

When analyzing the scaffold diversity of all selected compounds to understand the chemical diversity of the complete training data and not just the hits, random selection had the highest scaffold diversity of all selection strategies, while AD-CP had the most diverse scaffold selection of all active learning approaches, followed by Chemprop, Random Forest, AD-XGB, and XGBoost (p<0.0001 at 100 iterations, FIG. 23). As such, AD-CP not only finds the most chemically diverse hits, with potential to create multiple lead series to enable further development of distinct scaffolds, but this approach also enriches the scaffold diversity of “negative” training data to improve future compound selection. Although the deep learning-based models were not able to identify more potent inhibitors than the tree-based implementations here, a deep learning approach might be advantageous to identify more diverse hits.

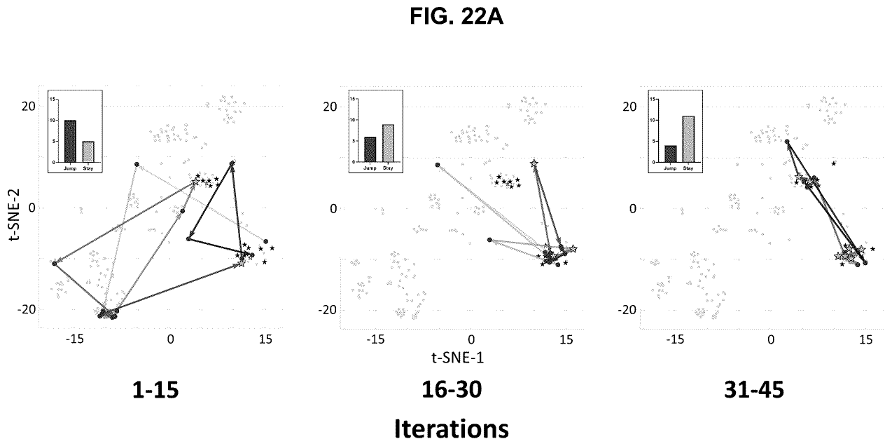

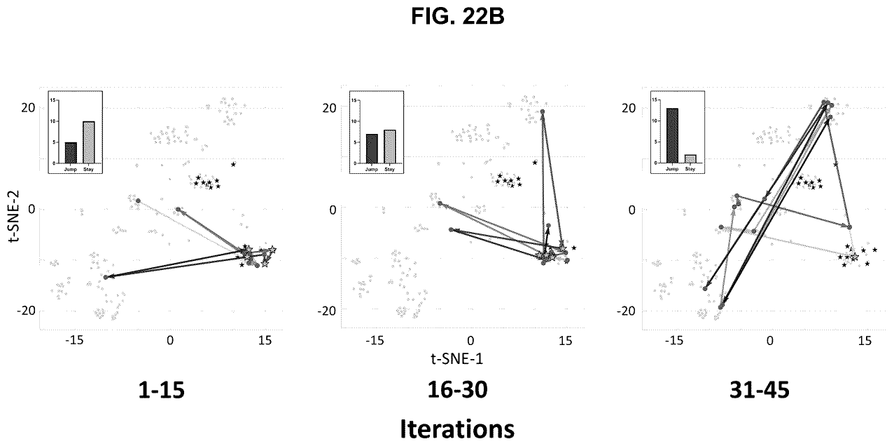

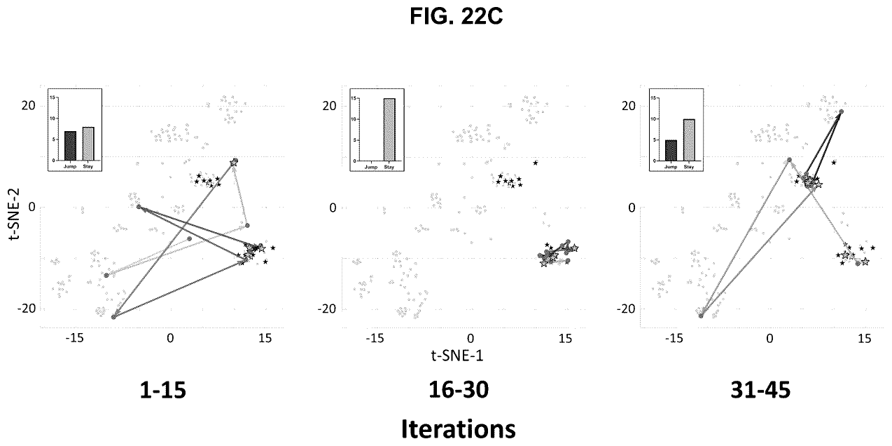

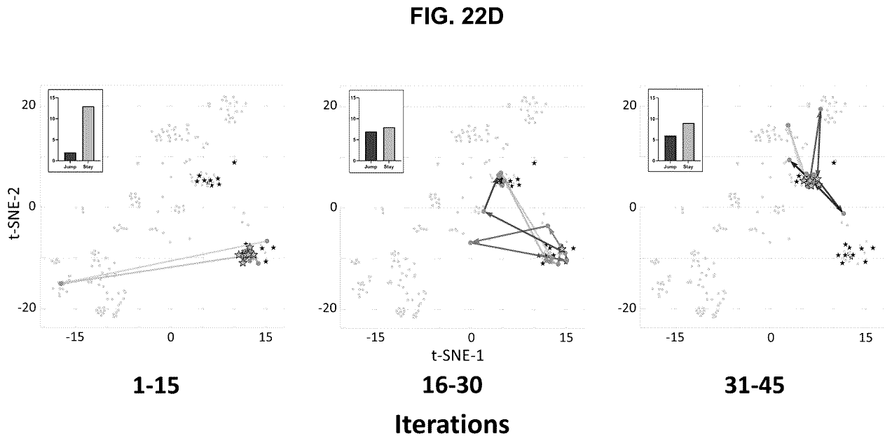

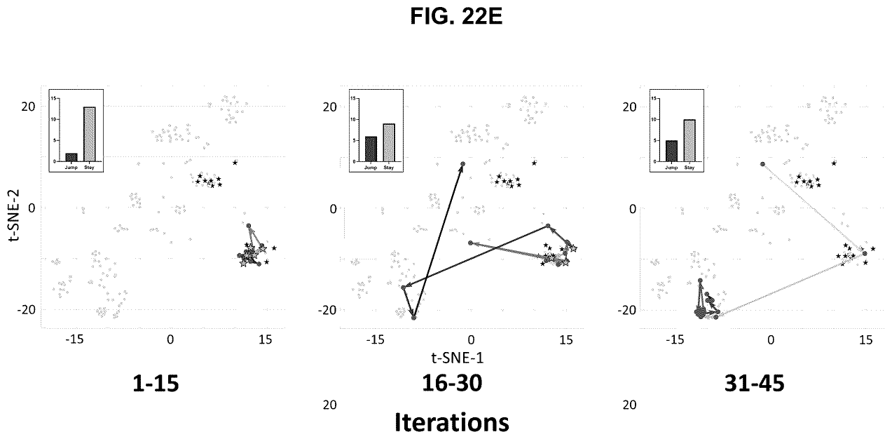

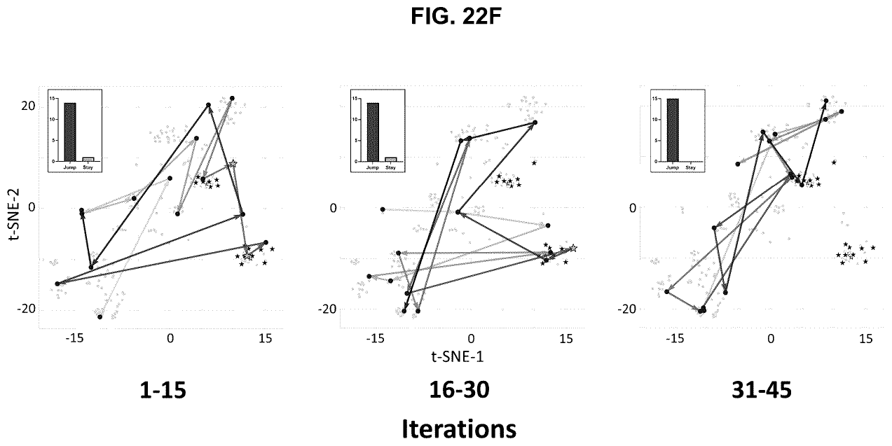

Analyzing Chemical Trajectories We next investigated how these models explored chemical space using t-SNE analysis based on radial chemical fingerprints of molecules selected during active learning. In the first learning iterations, AD-CP explored chemical space broadly and jumped between clusters (FIG. 22A). During 16-30 iterations, AD-CP showed a balanced behavior with equal numbers of jumps and staying within a cluster. After 30 iterations, AD-CP had identified all the relevant clusters of active compounds and largely stayed within these clusters to rapidly identify potent inhibitors. In contrast, Chemprop was more targeted at the beginning and exploited the one cluster where it could find potent inhibitors (FIG. 22B). After that, Chemprop started to become more explorative and was not able to identify all clusters of potent inhibitors even after 45 iterations of learning.

Similar to AD-CP, AD-XGB exhibited broad exploration jumping between clusters during the first learning iterations and identified a relevant cluster of potent compounds (FIG. 22C). During 16-30 iterations, AD-XGB stayed within this relevant cluster until after 30 iterations where it explored again to quickly identify another relevant cluster that it stayed within. XGBoost initially showed more targeted behavior where it exploited one cluster and then explored more during 16-30 iterations and discovered another relevant cluster it exploited (FIG. 22D). Random Forest immediately exploited the one cluster where it could find potent inhibitors, but after becoming more explorative it did not identify all clusters of potent inhibitors by 45 iterations of learning and instead focused on a cluster that did not contain any of the most potent molecules (FIG. 22E). As expected, random selection consistently showed broader exploration of chemical space than any active learning approach with consistent jumping between clusters for all iterations (FIG. 22F). Altogether, these results highlight how the ActiveDelta approach can guide models to select diverse chemistries while learning from initial exploration to effectively traverse chemical space (FIG. 22) to identify the most potent leads (FIG. 21A-D).

### Extrapolation to External Data

Motivated by the strong ability of ActiveDelta models to effectively navigate the learning spaces, we next sought to see how readily models trained on the selected molecules by active learning could generalize to new data. Using splits generated to mimic real-world medicinal chemistry project data sets (i.e., simulating learning from historic data to predict undiscovered “future” compounds), we evaluated all the models' performances after training on the 100 molecules they each selected from the learning set during exploitative active learning on the task of identifying novel hits (i.e., correctly predict the top ten percentile of the most potent compounds in the test sets). Across three repeats, AD-CP correctly identified the largest number of novel hit compounds (41.3%±18.5 on average), followed by AD-XGB (40.0%±18.9) and XGBoost (40.0%±20.4). Random Forest (37.9%±20.4) and single-molecule Chemprop (27.9%±18.7) had a weaker ability to identify potent inhibitors in the test set. AD-CP showed a significant improvement over Chemprop (p=2×10⁻²¹) but AD-XGB showed no statistically significant difference compared to XGBoost (p=0.9), possibly driven by the good performance of XGBoost alone. AD-CP was the only approach to correctly identify 100% of the hits within a test dataset while Random Forest peaked at 89%, AD-XGB and XGBoost peaked 88%, and Chemprop peaked at 83% of correctly identified hits.

In terms of chemical diversity of the novel hits identified in the test set, AD-CP also identified the most distinct novel hit scaffolds (3.3±1.7 scaffolds on average) followed by XGBoost (3.2±1.7), AD-XGB (3.1±1.6), Random Forest (2.9±1.7), and Chemprop (2.2±1.5). Similar to hit identification, AD-CP showed a significant improvement over Chemprop (p=8×10⁻²⁴) but AD-XGB showed no statistically significant difference compared to XGBoost (p=0.7).

Taken together, this data suggests that the Chemprop-based AD-CP is particularly powerful at building models that can generalize to new datasets and thereby will provide medicinal chemists with options to change utilized chemistries later in the project while utilizing knowledge generated from other molecules. Its ability to identify the most chemically-diverse hits will also make it a most useful tool to provide medicinal chemists with various lead series for further optimization.

Coinciding with increased enthusiasm for machine learning methods to support drug discovery, expanded use of adaptable laboratory automation will help support adaptive learning methods like active machine learning to become a cornerstone technology to guide molecular optimizations and discovery. The ActiveDelta approach for active learning may efficiently lead optimization pursuits by prioritizing the most promising candidates for subsequent evaluation and could be directly integrated into robotic chemical systems to generate more potent leads through iterative design. Beyond pharmaceutical design, we expect these methods to be easily deployable for other chemical endeavors to support material design and prioritization.

Although pairwise methods like ActiveDelta exhibit increased computational costs during active learning given their combinatorial expansion of training data, these extra datapoints benefit deep models' abilities to learn the underlying structure-activity-relationships more accurately and readily identify the most potent compounds of interest with varying scaffolds. Furthermore, as real-world experimentation often provides a larger bottleneck than computation, the use of more complex computational architectures with improved hit retrieval rates in place of faster, but less effective architectures should be more efficient overall for most projects.

Given the general notion of tree-based models' robustness to training on smaller datasets, AD-CP's ability to outcompete multiple tree-based models by only 100 iterations shows particular promise for the application of deep models for low data active learning that are typically particularly troublesome for data-hungry deep learning models. This improved performance translated to external datasets generated by mimicking the differences between early and late compounds from true pharmaceutical optimization projects, indicating the generalizability of this approach.

Applied to exploitative active learning, the ActiveDelta approach leverages paired molecular representations to predict molecular improvements from the best current training compound to prioritize molecules for training set expansion. Here, we have shown this approach allows both tree-based and deep learning-based models to rapidly learn from pairwise data augmentation in low data regimes to outcompete standard active learning implementations of state-of-the-art methods in identifying the most potent compounds during exploitative active learning (FIG. 21A-D) while selecting more diverse compounds and effectively navigating chemical space (FIG. 22). The deep models using this approach also more accurately identified hits in external test sets generated through simulated temporal splits, indicating its applicability and generalizability to novel chemical structures that would likely be encountered during medicinal chemistry projects. We believe that ActiveDelta and other pairwise approaches show particular promise for adaptive machine learning when training data hungry neural networks on limited data and can serve as accurate platforms to guide lead optimization and prioritization during drug development.

### Example 3

Major efforts are invested to optimize molecular potency during drug design. However, bottlenecks due to comparatively slow chemical syntheses during optimization often limit broader exploration of various chemical structures. To streamline synthesis and testing, molecular machine learning methods are increasingly employed to learn from historic data to prioritize the acquisition and characterization of new molecules.

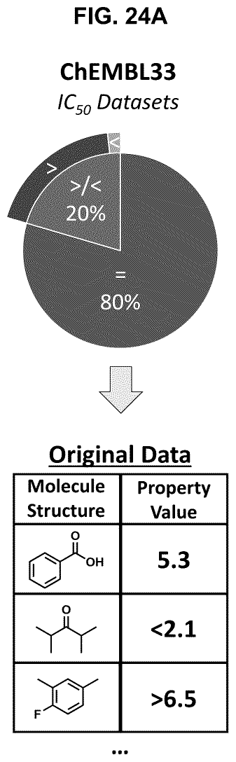

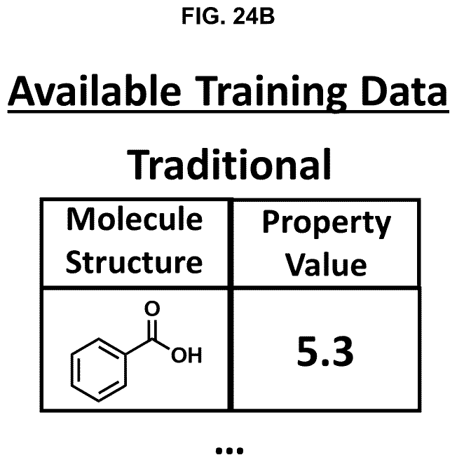

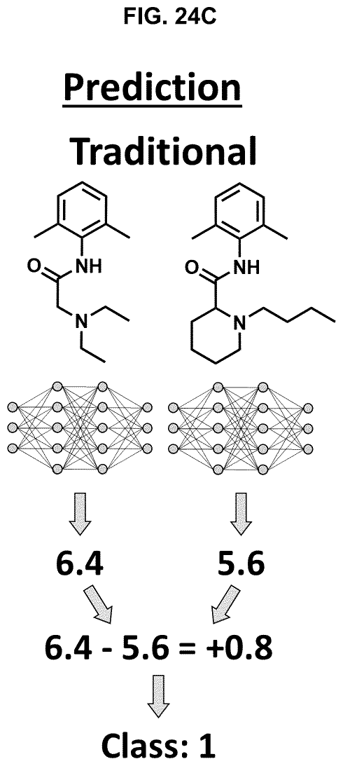

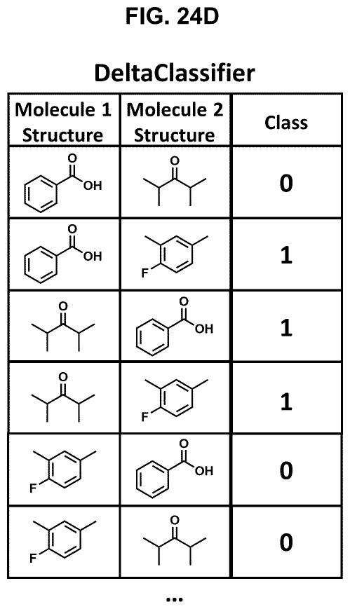

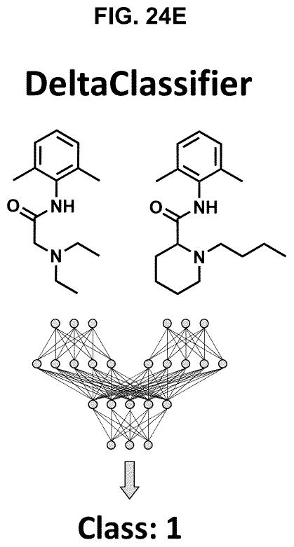

However, during data generation, a substantial fraction of molecules is still incompletely characterized, leading to reporting of bounded values in place of exact ones. Specifically, compound screening is often performed in a two-step process, where a large set of compounds is tested at a single concentration and only the most promising hits are further evaluated in full dose-response curves to determine IC₅₀ values. This results in a substantial fraction of datapoints not being annotated with their exact IC₅₀ values but instead with lower bounds. Conversely, upper bounds might be created through insufficient experimental resolution or solubility limits. In total, one fifth of the IC₅₀ datapoints in ChEMBL datasets are bounded values (FIG. 24A). This bounded data remains inaccessible to even the most powerful molecular machine learning algorithms, preventing machines from harnessing a substantial amount of available knowledge (FIG. 24B).

Furthermore, as the positive reporting bias imbalances available regression data towards the most potent compounds, incorporation of these compounds with more mild activity could help counteract skewed class proportions and provide valuable chemical diversity during training (Table 8).

Regression methods can be used to steer molecular optimization by predicting the potency of two molecules and comparing these predictions to select the molecule with higher predicted potency (FIG. 24C). However, as regression algorithms cannot train on bounded data, they only use a subset of the available training data with limited diversity (FIG. 24B, Table 8).

We previously showed that leveraging pairwise deep learning to simultaneously process two molecules and directly predict their absorption, distribution, metabolism, excretion, and toxicity (ADMET) property differences can improve predictive performance. We hypothesized that we could transform this pairing approach into a novel classification problem where the algorithm is tasked to predict which of the two paired molecules is more potent. This pairing would enable us to access bounded datapoints by pairing them with other molecules that are known to be more or less potent (FIG. 24D). Providing this data to a classification algorithm can create a predictive tool that directly contrasts molecules to guide molecular optimization while incorporating all of the available training data (FIG. 24E).

Here, we evaluate this paired machine learning approach, deemed DeltaClassifier, against the tree-based Random Forest, the gradient boosting method XGBoost, and the directed message passing neural network (D-MPNN) ChemProp, on predicting molecular potency improvements. Across 230 ChEMBL IC₅₀ datasets, both tree-based and neural network-based implementations of the DeltaClassifier concept exhibit improved performance over traditional regression approaches when predicting molecular improvements between molecule pairs. We believe that the DeltaClassifier approach and further extensions thereof will be able to access greater ranges of data to support drug design more accurately.

### Datasets

ChEMBL3322 was filtered for single organism/protein IC₅₀ of small molecules with molecular weights <1000 Da. We focused on datasets containing 300-900 datapoints to ensure sufficient data while preventing combinatorial explosion. Additionally, datasets were filtered to ensure no single IC₅₀ value (e.g., “>10,000 nM”, e.g., ChEMBL target ID 4879459) accounted for more than half of all datapoints which occurred in 9 datasets. Any invalid SMILES, duplicate molecule, or molecule labelled with an IC₅₀ value of 0 or N/A were removed. All IC₅₀ values were converted to pIC₅₀ from nanomolar concentrations. This data curation workflow resulted in 230 benchmarking datasets.

### Model Architecture and Implementation

For D×C, we built upon the same directed D-MPNN architecture as ChemProp given its efficient computation and competitive performance for molecular data. By building on this architecture, results were easily comparable to ChemProp and allowed for direct quantification of the benefits of our molecular pairing approach and the integration of bounded data. Two molecules formed an input pair for D×C, while ChemProp processed a single molecule to predict absolute potency that were then subtracted to calculate potency differences between two molecules and used classify IC₅₀ improvements using normalized predicted differences as model confidence in the classification problem (FIG. 24C). In contrast, D×C directly learned and classified IC₅₀ improvements by training on input pairs and their potency differences (FIG. 24E). For ChemProp and D×C, molecules were described using atom and bond features as previously implemented. For D×C, molecular graphs were first individually converted into a latent representation by passing through a directed D-MPNN and then the latent representations of the two molecules within a pair were subsequently concatenated to generate a paired representation. This concatenated embedding was then passed through a second neural network of linear feed forward layers for potency prediction. ChemProp was set as ‘regression’ while D×C was set as ‘classification.’ As ChemProp was set for regression, it could only be trained on exact values within the training set. Both deep learning models were implemented with default parameters and aggregation=‘sum’ using the PyTorch deep learning framework. For the traditional ChemProp implementation, “number of molecules” is set to 1 while for D×C it is set to 2 to process molecular pairs23. We previously optimized the number of epochs for molecular paired data2 and accordingly set epochs=5 for D×C and epochs=50 for ChemProp.

For Random Forest and XGBoost models, molecules were described using radial chemical fingerprints (Morgan circular fingerprint, radius 2, 2048 bits, rdkit.org). Random Forest regression models were set with 500 trees. Both Random Forest and XGBoost were implemented with default parameters in scikit-learn. For Random Forest and XGBoost, each molecule was processed individually such that predictions were made solely based on the fingerprint of a single molecule. Regression models were only able to be trained on exact values within the training set. For developing ΔCL, XGBoost models were chosen due to XGBoost's established GPU-accelerated implementation. For the ΔCL, fingerprints for paired molecules were concatenated to form paired molecular representations to directly train on and classify potency improvements using the classification implementation of XGBoost.

For all standard regression algorithms (ChemProp, Random Forest, XGBoost), predicted potency differences were calculated by subtracting predictions for the two molecules within a pair and using normalized predicted differences as model confidence for classification. Each predicted difference was normalized as xnorm=[x−xmin]/[xmax−xmin], where x is the difference in predicted potency between two molecule pairs, xmax is the maximum predicted potency differences between all pairs of the test dataset, and xmin is the minimum predicted potency difference between all pairs of the test dataset. This normalization creates a normalized xnormϵ[0, 1] that is larger for molecule pairs with larger potency differences and therefore serves as a surrogate predictive confidence measure to enable ROCAUC calculations.

### Model Evaluation

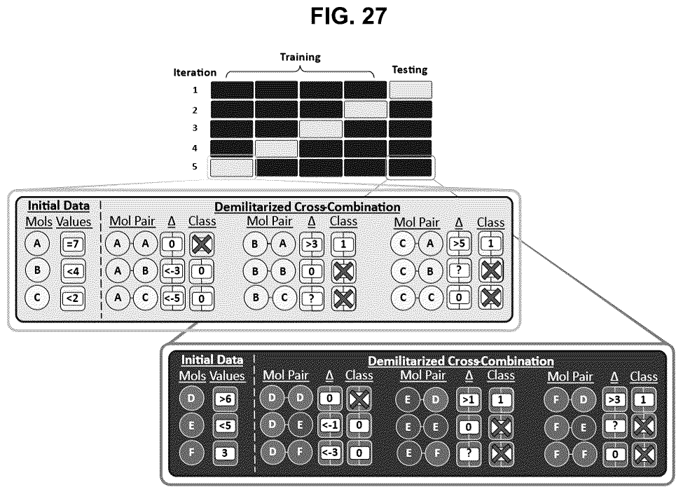

For evaluating the impact of demilitarization on the training of DeltaClassifiers and evaluating with modified test sets, models were evaluated using 1×10-fold (sklearn). For comparisons with traditional approaches, models were evaluated using 3×10-fold cross-validation. In all cross validations, models were evaluated with accuracy, F1 score, and Area Under the Receiver Operating Characteristic Curve (ROCAUC). To prevent data leakage, data was first split into train and test sets during cross-validation prior to cross-merging to create molecule pairings (FIG. 27). This ensured each molecule was only present in pairs made in the training or the test set but never both. If it was unknown if the potency was improved for a molecular pair (e.g., both molecules' potencies are denoted as upper bounds), the pair was removed. Additionally for demilitarized assessments and training, if the difference was less than 0.1 pIC₅₀, the pair was removed to account for experimental noise and non-statistically significant potency differences. For assessments of training on only exact values, any datapoint denoted as ‘>’ or ‘<’ was removed. Scaffold analysis, analysis of the influence of bounded datapoints on model performance, and additional test sets (without filtering of low pIC₅₀ differences or without filtering but removal of same molecule pairs) were made using cross-validation splits with a random state=1. Z-scores were calculated using scipy and modified z-scores (Mi) were calculated using the following equation:

$${M}_{i} = \frac{0.6745 ( {x}_{i} - {\tilde{x}}_{i} )}{M A D}$$

wherein \tilde{x} i is the median and MAD is the median absolute deviation24.

Statistical comparisons were performed using the non-parametric Wilcoxon signed-rank test (p<0.05) when comparing across the 230 datasets or across models and performed as paired t-tests (p<0.05) for cross-validation repeats of a single dataset. Violin plots were made in GraphPad Prism 10.2.0 while scatterplots were made using matplotlib.

### Influence of Bounded Data on Performance

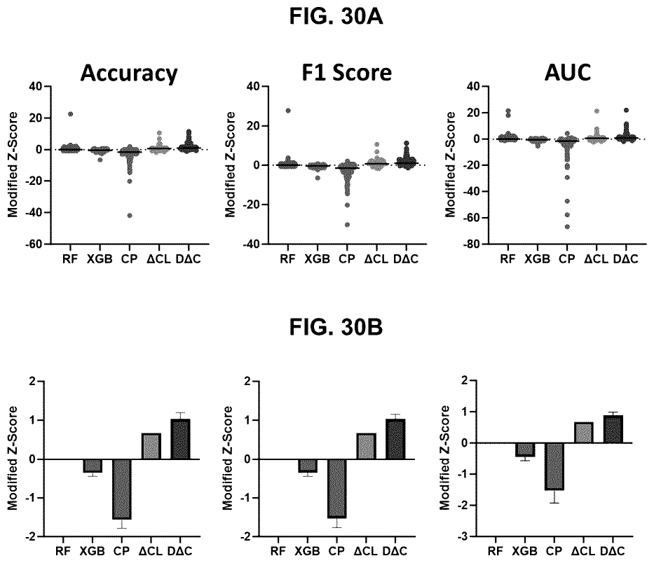

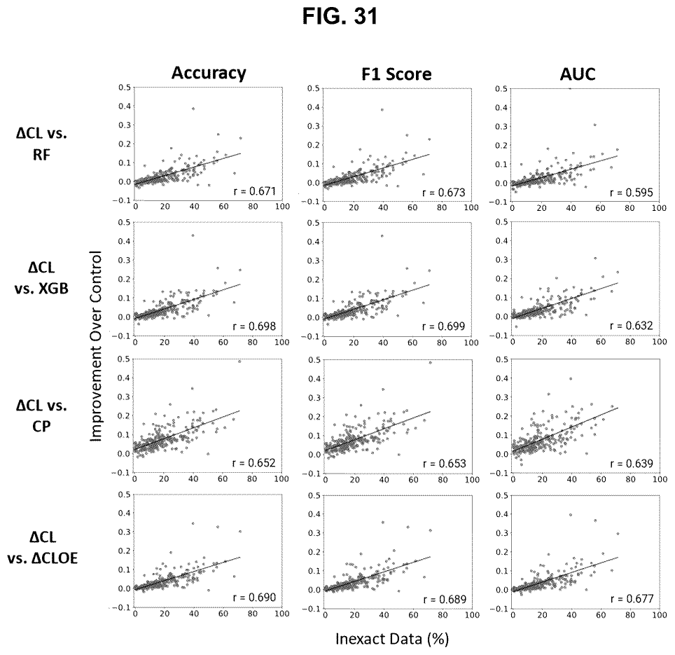

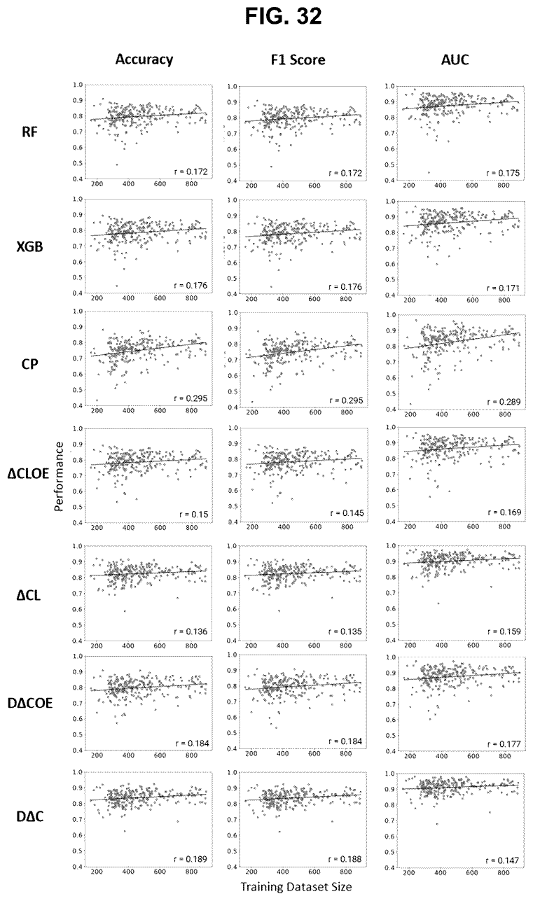

Next, we sought to determine how the number of bounded datapoints in training data affects the improvement of DeltaClassifiers over traditional methods. The number of bounded datapoints within the training datasets correlated with the improvement of D×C (Pearson's r=0.58-0.75, FIG. 30) and ΔCL (Pearson's r=0.56-0.70, FIG. 31) over traditional regression models and training with only exact values. Therefore, our approach is most powerful if large amounts of bounded data is available that is not normally available to regression approaches. Importantly, these correlations are stronger (p=0.0005) than the weaker correlations seen between dataset size and model performance (Pearson's r=0.14-0.30, FIG. 32), indicating that the benefit of the larger amounts of bounded data are not simply driven by larger dataset sizes. This evaluation suggests that the DeltaClassifier methods can be particularly helpful for datasets with large amounts of bounded datapoints.

### Scaffold Analysis

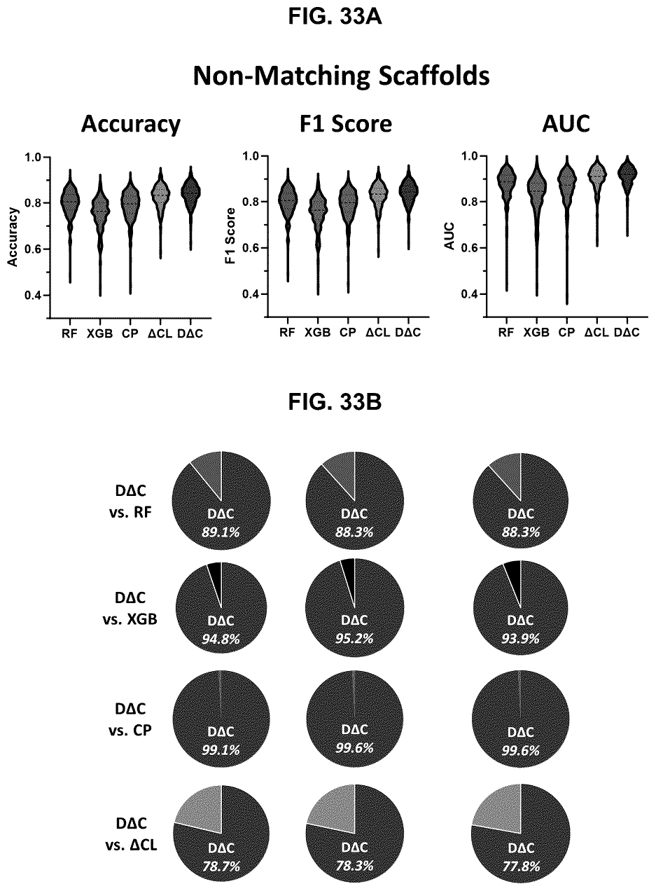

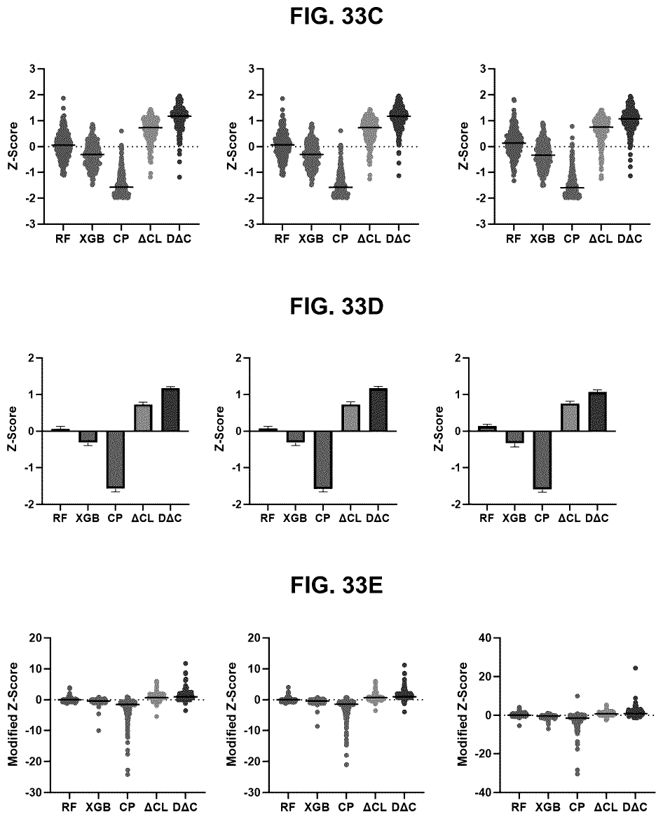

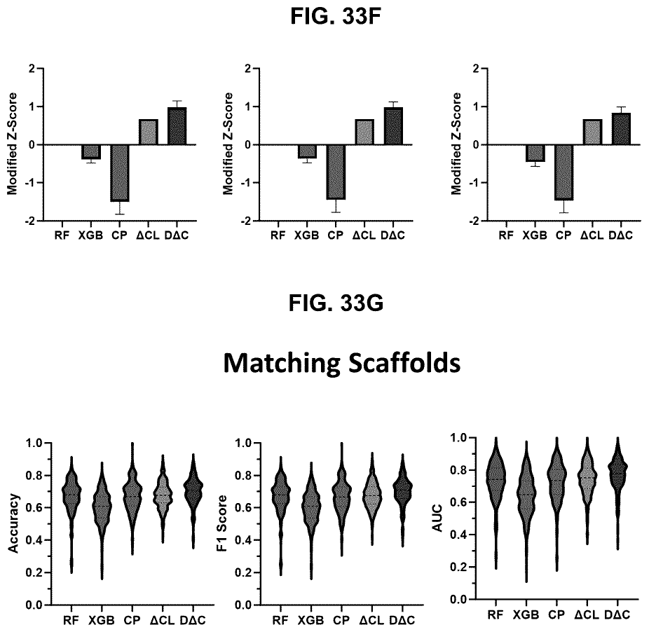

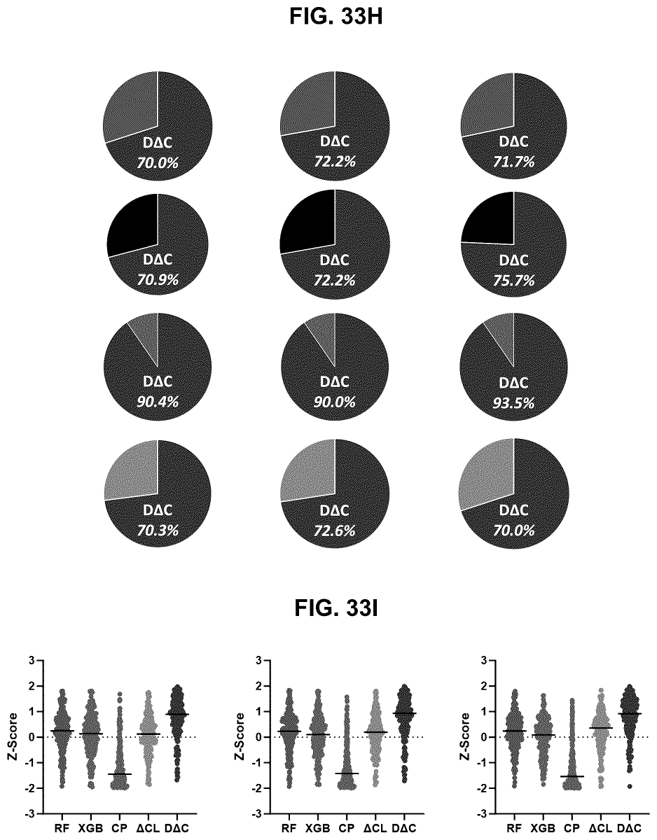

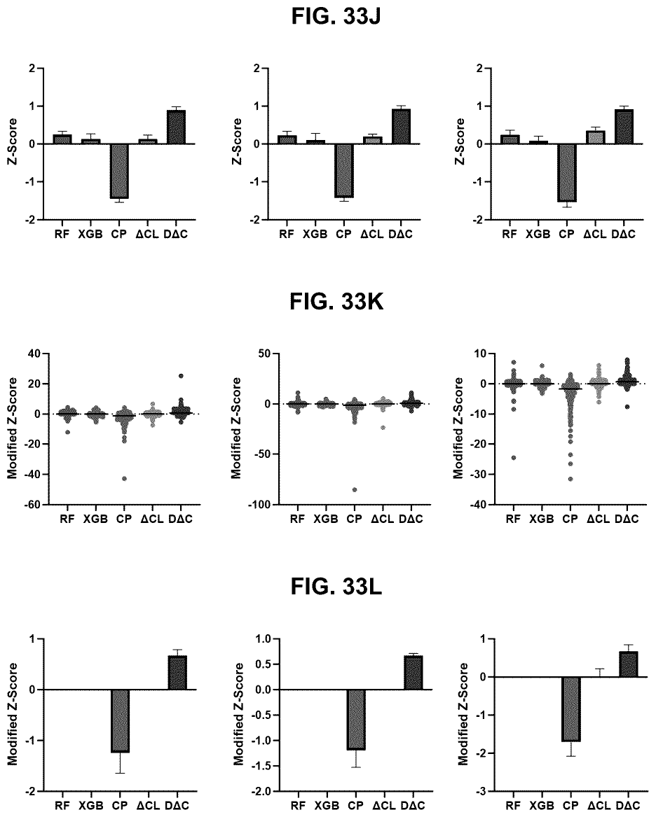

Next, we evaluated which model could most accurately predict potency improvements for pairs with either the same or different scaffolds, thereby evaluating the ability of the DeltaClassifier approach to support focused structure optimization or scaffold-hopping. After splitting test fold pairs into two separate groupings (shared or differing Murcko scaffolds, respectively), we evaluated model performance on both test sets after training the algorithms on the complete training folds containing pairs of both groupings. Gratifyingly, D×C outperformed traditional approaches both on predicting potency differences between molecules with different scaffolds (p<0.0001, Tables 17-18, FIG. 33A-F) and between molecules with same scaffolds (p<0.0001, Tables S19-20, FIG. 33G-L), outperforming classical approaches on both use-cases. D×C achieved highest median Z scores across all datasets (FIG. 33C/I) and showed highest average rank compared to all other investigated methods (Table 18/20). This implies that D×C could potentially be used both for fine-tuned compound optimization on the same scaffold while also enabling more drastic scaffold-hopping into new compound classes while optimizing potency.

Training Deep Models with Bounded Data

We hypothesized that using a paired approach to directly train on and classify molecular potency improvements would not only allow for the incorporation of bounded datapoints into training, but also improve overall model performance. To evaluate this hypothesis, we created a novel machine learning task wherein molecular pairs function as the datapoints instead of individual molecules (FIG. 24D). The target variable is created by comparing the potency of the paired molecules and assigning class “1” when the second molecule is more potent and “0” otherwise (i.e., the first molecule is more potent or both molecules have equal potency). In other words, our classification tool answers the question “Is the second molecule more potent than the first molecule?” (FIG. 24D). If it is unknown if the potency is improved (e.g., both IC₅₀ values in the pair are upper bounds), the pair is removed. Further, to account for experimental noise, we used a ‘demilitarized’ training approach where only molecular pairs with differences greater than 0.1 pIC₅₀ were used for training and testing. This is to avoid training the model on potentially statistically insignificant potency differences as well as excluding data where the label could easily “flip” due to experimental uncertainty. Following filtering, a machine learning model capable of accepting two molecular inputs can be trained on this data to classify if the second molecule exhibits an improvement in potency over the first molecule.

To evaluate these models, we used cross-validation to randomly split our ChEMBL benchmarking datasets into training and testing sets (FIG. 27). Within each split, molecules were cross-merged to form all possible pairs and classified according to their potency difference while filtering inconclusive and uncertain pairs as described above.

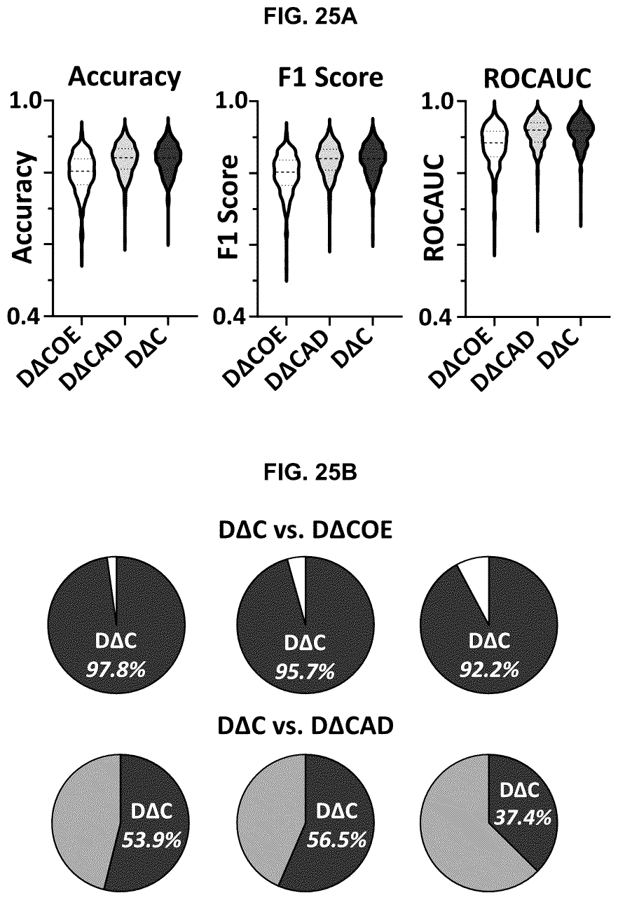

First, we tested the performance of a D-MPNN-based version of the DeltaClassifier (DeepDeltaClassifier, D×C) across 230 IC₅₀ datasets from ChEMBL by building on ChemProp 5 to evaluate if a state-of-the-art molecular machine learning approach could accurately solve this task. Across 230 IC₅₀ datasets, we found promising performance of this new approach for classifying molecular potency improvements with an average ROCAUC of 0.91±0.04, ranging from 0.68-0.98, and average accuracy of 0.84±0.04, ranging from 0.62-0.92, (FIG. 25, Table 8, Table 10). This encouraging performance highlighted the ability of the D-MPNN machine learning model to accurately solve our novel task, with high potential to serve as a guiding tool for molecular optimization tasks.

To assess the impact of our demilitarization, we analogously implemented D×C but trained on all data without filtering pairs with potency differences smaller than 0.1 pIC₅₀ (D×CAD). D×C and D×CAD exhibited overall comparable performance with no significant difference between D×C and D×CAD for accuracy (p=0.054), slight improvement for D×C for F1 (p=0.002), and slight improvement for D×CAD for AUC (p=0.003, FIG. 25, Table 10,) when evaluating on demilitarized test sets. Similar trends were observed for test sets with all pairs included (Table 11) and all pairs except the same molecule pairs that are always classified as “0” (Table 12). As D×C accounts for experimental noise by training only on pairs with larger differences and shows no drop in performance with fewer training datapoints, we believe that our demilitarized approach is an appropriate method for training to classify molecular potency improvements.

Since it is known that IC₅₀ data has substantial variability, we also assessed whether stricter (i.e., larger) thresholds would provide further benefits to the model. To this end, we created additional DeltaClassifier models that were trained on potency differences larger than 0.5 pIC₅₀ and 1.0 pIC₅₀. When evaluated on a test set that included all data to provide a uniform evaluation, these larger buffer zones led to a decrease in performance (p<0.0001, Table 13) compared to D×C. This continued to be true when trivial same molecule pairs that are always classified as “0” were removed from this test set (p<0.0001, Table 14). This data suggests that our demilitarization of 0.1 pIC₅₀ is sufficient to account for experimental error and potentially benefits from more data compared to stricter thresholds.

Finally, to determine if training on bounded datapoints improved performance compared to just training on the exact IC₅₀ values, we analogously implemented the D×C but trained only on molecular pairs with exact values (D×COE). D×C significantly outperformed D×COE (p<0.0001) across all metrics (FIG. 25, Table 10). Similar trends were observed for test sets without filtering of low pIC₅₀ differences (Table 11) and without filtering but removal of same molecule pairs (Table 12) highlighting how training on bounded datapoints can improve overall model performance. This suggests that our novel machine learning task can enable models to incorporate additional data into the model that significantly boosts performance. We next set out to evaluate whether other algorithms beyond D-MPNN can solve this new DeltaClassifier task and how our models performed compared to state-of-the-art molecular machine learning models.

### Tree-Based DeltaClassifiers

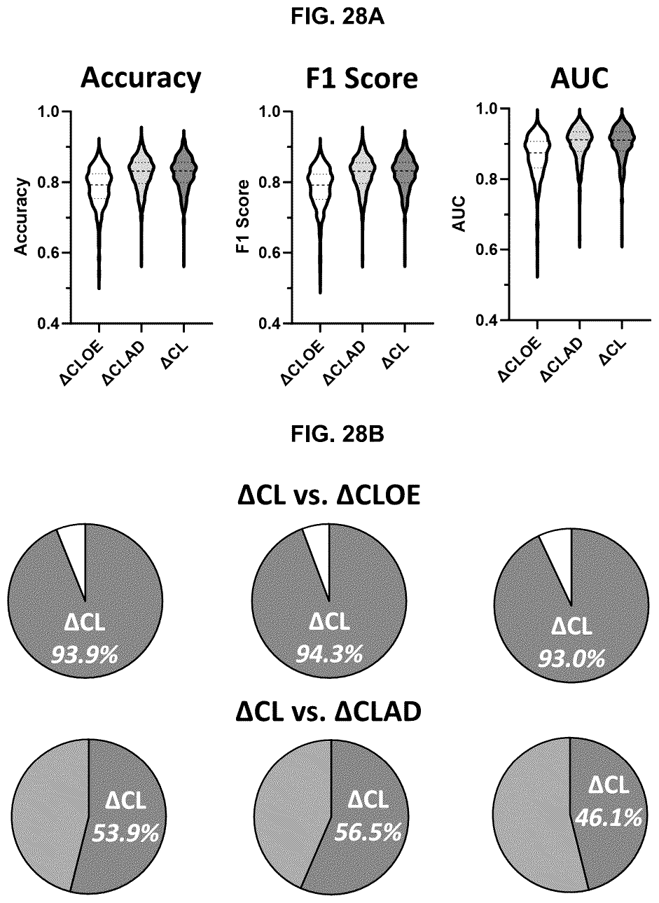

In addition to implementing MPNN-based DeltaClassifiers, we also implemented XGBoost-based classifiers to evaluate how tree-based models would perform on this approach. XGBoost was selected due to its readily available GPU acceleration4, which can speed up calculation on large datasets created through our pairing. Due to their increased computational efficiency, we further refer to these XGBoost-based DeltaClassifiers as DeltaClassifierLite. Like the deep models, DeltaClassifierLite trained on demilitarized data (ΔCL) significantly outperformed training on only exact values (ΔCLOE, p<0.0001, FIG. 28, Table 10 Supplementary File 2). Overall comparable performance was observed between ΔCL and an approach that used all data without filtering for small property differences (ΔCLAD) with no statistically significant difference for accuracy (p=0.3), slight improvement for ΔCL for F1 (p=0.002), and no significant difference for AUC (p=0.7, FIG. 28, Table 10). Analogously to the D-MPNN-based implementations, similar trends were observed for a test set with all pairs included (Table 11) and all pairs except same molecule pairs (Table 12). Altogether, these results support that our new classification task can be solved by both deep D-MPNNs and tree-based XGBoost algorithms, although overall superior performance by the D-MPNN-based implementation compared to the tree-based version suggests the utility of deep neural networks to predict potency improvements between molecular derivatives.

TABLE 8
Results for 3 × 10-Fold Cross-Validation Tested on Demilitarized Data.
Average and standard deviation of accuracy, F1 score, and AUC are presented
for five models following 3 × 10-fold cross-validation for 230 datasets.
Highest statistically significant performances across all models are bolded.

| Model Type | Model | Accuracy | F1 Score | ROCAUC |
|---|---|---|---|---|
| Traditional | Random Forest | 0.80 ± 0.06 | 0.80 ± 0.06 | 0.87 ± 0.06 |
| | XGBoost | 0.79 ± 0.06 | 0.79 ± 0.06 | 0.86 ± 0.07 |
| | Chem-Prop | 0.75 ± 0.07 | 0.75 ± 0.07 | 0.82 ± 0.08 |
| DeltaClassifier | ΔCL | 0.82 ± 0.05 | 0.82 ± 0.05 | 0.90 ± 0.05 |
| | DΔC | 0.84 ± 0.04 | 0.84 ± 0.04 | 0.91 ± 0.04 |

Comparisons with Traditional Approaches

Next, we investigated if DeltaClassifiers would exhibit improved performance over using traditional regression approaches when predicting potency improvements (FIG. 27C). We compared our D×C and ΔCL approach against two state-of-the-art tree-based machine learning algorithms, Random Forest and XGBoost, and a D-MPNN graph-based deep learning algorithm (ChemProp). For these three state-of-the-art regression models, models were trained on the training molecules with exact values and then used to predict absolute potency values of each test set molecule. Afterwards, potency improvements of pairs were inferred by subtracting the potency predictions to determine which compound was expected to be more potent. Instead of simply designating a positive difference as a positive class and a negative difference as a negative class, we normalized the predicted differences between molecules to create a proxy for model confidence.

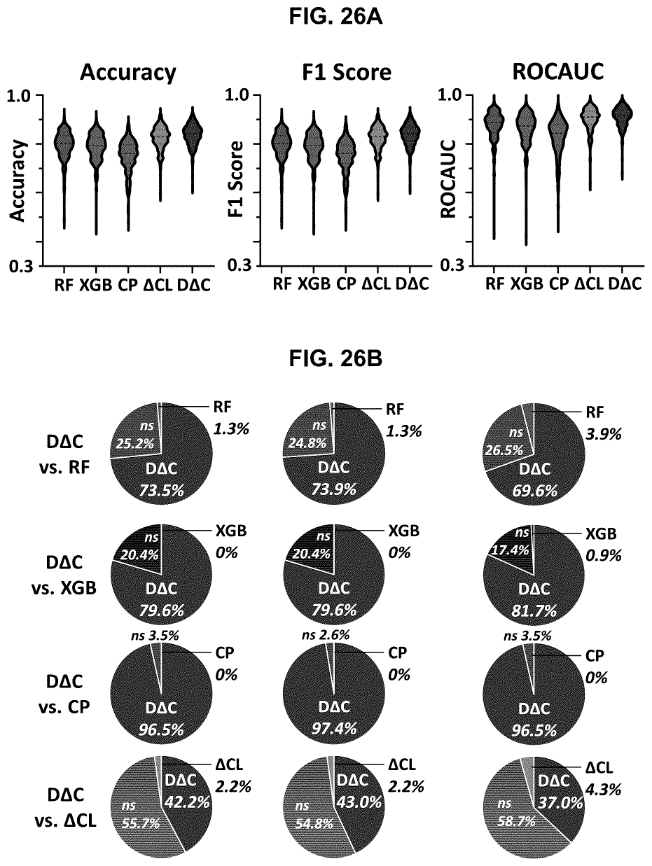

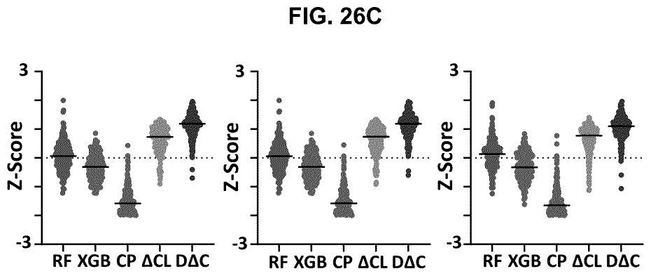

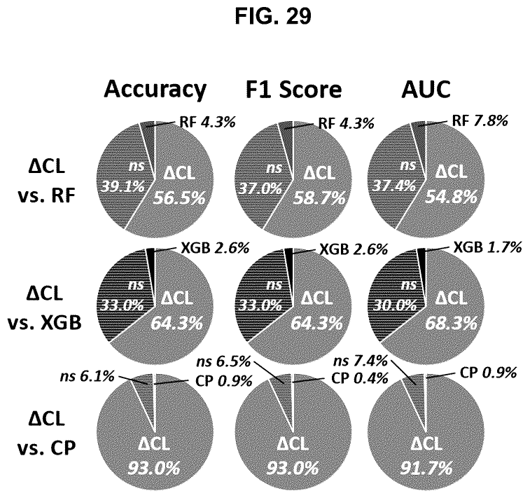

In terms of accuracy, F1 Score, and ROCAUC, D×C showed a statistically significant improvement over all other methods (p<0.0001, Table 8, FIG. 26A). At the level of individual datasets, D×C outcompeted all traditional methods in at least 69% of datasets and was competitive in at least 96% of datasets for all metrics (p<0.05, FIG. 26B). The largest benefit was seen over the regression version of ChemProp, wherein D×C outcompeted at least in 222/230 datasets for all metrics and was not statistically significantly worse on any dataset, highlighting the particular benefit of combinatorial data expansion from molecular pairing for data-hungry deep models. ΔCL also outcompeted the traditional methods in most datasets across all metrics, but at a consistently slightly lower percentage than D×C (p<0.05, FIG. 29) across all metrics. When evaluating all five models on the level of each dataset D×C showed the highest median Z-score followed by ΔCL across all metrics (FIG. 26C) and the same trends were observed for modified Z-scores (FIG. 30). In terms of rank, D×C showed the highest average rank for accuracy (1.29±0.65), followed by ΔCL (2.13±0.72), Random Forest (2.91±0.80), XGBoost (3.84±0.60), and ChemProp (4.84±0.56) with similar trends for F1 score and AUC (Table 15). These results attest to the superior performance of DeltaClassifiers compared to traditional regression methods when predicting potency improvements between molecules.

D×C also outcompeted all other approaches for test sets without filtering of low pIC₅₀ differences (p<0.0001, Table 11) and without filtering but removal of same molecule pairs (p<0.0001, Table 12). When evaluated on a test set only containing data with exact values and no same molecular pairs, D×C still outcompeted ChemProp, XGBoost, and ΔCL (p<0.0001, Table 16), but exhibited similar performance compared to Random Forest in terms of accuracy (p=0.3) and F1 score (p=0.07), and lower performance in ROCAUC (p<0.0001, Table 16). This further attests to the strength of the DeltaClassifier approach to benefit from incorporating bounded potency values while the pairing alone might not inherently benefit performance compared to robust tree-based models. This motivated us to investigate the impact of the amount of bounded data on DeltaClassifier performance.

Here, we developed, validated, and characterized a molecular learning approach, DeltaClassifier, that directly trains on and classifies potency improvements of molecular pairs. Across 230 datasets from ChEMBL, tree-based and deep DeltaClassifiers significantly improve performance over traditional regression approaches to classify IC₅₀ improvements between molecules. This method benefits deep models even more than tree-based models, highlighting the particular advantage of combinatorial data expansion for data hungry deep models. DeltaClassifiers showed even greater improvements for datasets with more bounded data, suggesting that this method could be particularly beneficial for datasets with greater uncertainty for example during early stages of drug discovery campaigns. Our D-MPNN-based DeltaClassifier outperformed all other methods for molecular pairs with shared and differing scaffolds, highlighting the utility of this approach for both precise compound optimization and more drastic chemical derivatizations.

DeltaClassifiers can benefit from increased training datapoints and cancellation of systematic errors within datasets through pairing while directly learning potency differences. This data augmentation also allows for expedited model convergence2, leading to improved performance for DeltaClassifiers after only 5 epochs compared to standard ChemProp trained for 50 epochs (Table 8). Admittingly, paired methods are most efficiently applied to small or medium-sized datasets (<1000 datapoints) as their combinatorial expansion of training data leads to increased computational costs for each epoch. Altogether, the improved performance exhibited by DeltaClassifier over established methods across these benchmarks showcase its potential for potency classification with clear prospects for further improvements.

There are several related, powerful approaches to classify and compare molecular pairs. Siamese neural networks consider two inputs and tandemly use the same weights to compare inputs through contrastive learning. They have been applied within the field of drug discovery to predict molecular similarity, bioactivity, toxicity, drug-drug interactions, relative free energy of binding, and transcriptional response similarity. These models have shown particular promise when trained on compounds with high similarity. Although these models are similarly tailored to directly compare molecular pairs, they are not inherently constructed to utilize bounded data and typically rely upon similarity metrics, such as cosine similarity, to determine distance between classes. There is also precedence of using bipartite ranking of chemical structures to incorporate qualitative data with quantitative data when predicting molecular properties. For example, kernel-based ranking algorithms that minimize a ranking loss function rather than a classification or regression loss have been used for molecular ranking. More recently, classifiers have been trained upon molecular improvements to rank candidates for SARS-COV-2 inhibition. Instead of incorporating bounded values as we do for DeltaClassifiers, these approaches added labelled data (i.e., ‘inactive’) to regression data by considering all compounds with no measurable IC₅₀ as less active than active compounds. Ranking compounds from the same assay has also been implemented to counteract inter-assay variability. These existing classification approaches for molecular improvements should be synergistic with our DeltaClassifier approach. Together, we believe that these methods show great promise to supplement or replace machine learning methods currently implemented for intricate molecular optimizations, chiefly when relying upon smaller datasets with bounded or noisy data.

As generating valuable biological data is expensive, there is a clear need for novel methods to integrate all available data into machine learning training. We present DeltaClassifier, a novel classification approach that accesses traditionally inaccessible bounded datapoints to guide potency optimizations through directly contrasting molecular pairs. Given DeltaClassifiers' significant improvement in identifying potency improvements compared to traditional regression approaches, we believe that DeltaClassifier and subsequent extensions stand to accurately guide potency optimizations in the future. This method is poised to prioritize the most promising next pharmaceutical candidates and could be directly incorporated into adaptive robotic platforms for automated discovery campaigns. Beyond its utility in drug development, we believe DeltaClassifier can be implemented for material selection and optimization, thereby improving efficiency and quality for many important biological and chemical optimization tasks.

TABLE 9
Potency Distribution of Available IC₅₀ Data. Percentages
of exact, bounded, and all datapoints that are above
or below 1 μM in potency and average number of unique scaffolds
in each dataset for our 230 IC₅₀ datasets.

| | Exact | Bounded | All |
|---|---|---|---|
| >1 μM | 63.4% | 12.8% | 54.4% |
| <1 μM | 36.6% | 87.2% | 45.6% |
| Average Unique Scaffolds | 167 | 52 | 208 |

TABLE 10
Results for 1 × 10-Fold Cross-Validation Tested on Demilitarized Data. Average
and standard deviation of accuracy, F1 score, and AUC are presented for all models following
removal of molecular pairs with differences greater than 0.1 plC₅₀ in the test set.
Highest statistically significant performances across all models are underlined.
Highest statistically significant performances within each model family (traditional
models, tree-based Δ classifiers, and deep Δ classifiers) are bolded.

| | Traditional Methods | | | Tree-Based Δ Classifiers | | | Deep Δ Classifiers | | |
|---|---|---|---|---|---|---|---|---|---|
| Metric | RF | XGB | CP | ΔCLOE | ΔCLAD | ΔCL | DΔCOE | DΔCAD | DΔC |
| Accuracy | 0.795 ± | 0.785 ± | 0.748 ± | 0.785 ± | 0.824 ± | 0.824 ± | 0.797± | 0.836 ± | 0.836 ± |
| | 0.057 | 0.061 | 0.069 | 0.059 | 0.048 | 0.048 | 0.056 | 0.045 | 0.045 |
| F1 Score | 0.795 ± | 0.785 ± | 0.748 ± | 0.783 ± | 0.823 ± | 0.824 ± | 0.796 ± | 0.835 ± | 0.836 ± |
| | 0.057 | 0.061 | 0.069 | 0.062 | 0.048 | 0.048 | 0.059 | 0.045 | 0.045 |
| ROCAUC | 0.874 ± | 0.861 ± | 0.825 ± | 0.863 ± | 0.901 ± | 0.901 ± | 0.874 ± | 0.910 ± | 0.910 ± |
| | 0.063 | 0.069 | 0.081 | 0.065 | 0.048 | 0.047 | 0.060 | 0.042 | 0.042 |

TABLE 11
Results for 1 × 10-Fold Cross-Validation Tested on All Datapoints. Average and
standard deviation of accuracy, F1 score, and ROCAUC are presented for all models.
Highest statistically significant performances across all models are underlined. Highest
statistically significant performances within each model family (traditional models,
tree-based Δ classifiers, and deep Δ Traditional Methods classifiers) are bolded.

| | Traditional Methods | | | Tree-Based Δ Classifiers | | | Deep Δ Classifiers | | |
|---|---|---|---|---|---|---|---|---|---|
| Metric | RF | XGB | CP | ΔCLOE | ΔCLAD | ΔCL | DΔCOE | DΔCAD | DΔC |
| Accuracy | 0.785 ± | 0.776 ± | 0.742 ± | 0.770 ± | 0.807 ± | 0.804 ± | 0.780 ± | 0.817 ± | 0.815 ± |
| | 0.055 | 0.058 | 0.065 | 0.056 | 0.048 | 0.048 | 0.054 | 0.045 | 0.045 |
| F1 Score | 0.780 ± | 0.770 ± | 0.736 ± | 0.764 ± | 0.803 ± | 0.801 ± | 0.775 ± | 0.813 ± | 0.812 ± |
| | 0.057 | 0.060 | 0.068 | 0.060 | 0.050 | 0.049 | 0.058 | 0.047 | 0.047 |
| ROCAUC | 0.857 ± | 0.845 ± | 0.810 ± | 0.848 ± | 0.886 ± | 0.885 ± | 0.859 ± | 0.896 ± | 0.895 ± |
| | 0.064 | 0.069 | 0.080 | 0.065 | 0.050 | 0.050 | 0.060 | 0.046 | 0.045 |

TABLE 12
Results for 1 × 10-Fold Cross-Validation Tested Without Same Molecule Pairs.
Average and standard deviation of accuracy, F1 score, and ROCAUC are presented
for all models following removal of molecular pairs of the same molecule in the
test set. Highest statistically significant performances across all models are
underlined. Highest statistically significant performances within each model family
(traditional models, Δ classifiers, and deep Δ classifiers) are bolded.

| | Traditional Methods | | | Tree-Based Δ Classifiers | | | Deep Δ Classifiers | | |
|---|---|---|---|---|---|---|---|---|---|
| Metric | RF | XGB | CP | ΔCLOE | ΔCLAD | ΔCL | DΔCOE | DΔCAD | DΔC |
| Accuracy | 0.781 ± | 0.771 ± | 0.736 ± | 0.772 ± | 0.811 ± | 0.81 ± | 0.784 ± | 0.822 ± | 0.822 ± |
| | 0.057 | 0.060 | 0.067 | 0.058 | 0.049 | 0.049 | 0.055 | 0.046 | 0.046 |
| F1 Score | 0.780 ± | 0.770 ± | 0.736 ± | 0.769 ± | 0.809 ± | 0.81 ± | 0.782 ± | 0.821 ± | 0.821 ± |
| | 0.057 | 0.060 | 0.068 | 0.061 | 0.050 | 0.049 | 0.058 | 0.047 | 0.047 |
| ROCAUC | 0.861 ± | 0.848 ± | 0.813 ± | 0.850 ± | 0.889 ± | 0.889 ± | 0.862 ± | 0.899 ± | 0.898 ± |
| | 0.064 | 0.070 | 0.081 | 0.066 | 0.050 | 0.050 | 0.061 | 0.045 | 0.045 |

TABLE 13
Demilitarization Parameter Optimization for 1 × 10-
Fold Cross-Validation Tested. Average and standard deviation
of rankings of z-scores for accuracy, F1 score, and ROCAUC
are presented for all models. Highest statistically significant
performances across all models are bolded.

Δ Classifiers

| Metric | 0.1 pIC50 | 0.5 pIC50 | 1.0 pIC50 |
|---|---|---|---|
| Accuracy | 0.815 ± 0.045 | 0.812 ± 0.046 | 0.807 ± 0.049 |
| F1 Score | 0.812 ± 0.047 | 0.81 ± 0.048 | 0.804 ± 0.05 |
| ROCAUC | 0.895 ± 0.045 | 0.893 ± 0.047 | 0.89 ± 0.051 |

TABLE 14
Demilitarization Parameter Optimization for 1 × 10-Fold
Cross-Validation Tested Without Same Molecule Pairs. Average
and standard deviation of rankings of z-scores for accuracy,
F1 score, and ROCAUC are presented for all models. Highest statistically
significant performances across all models are bolded.

Δ Classifiers

| Metric | 0.1 pIC50 | 0.5 pIC50 | 1.0 pIC50 |
|---|---|---|---|
| Accuracy | 0.822 ± 0.046 | 0.819 ± 0.047 | 0.814 ± 0.05 |
| F1 Score | 0.821 ± 0.046 | 0.819 ± 0.047 | 0.813 ± 0.05 |
| ROCAUC | 0.898 ± 0.045 | 0.897 ± 0.047 | 0.893 ± 0.051 |

TABLE 15
Ranking of Model Performance for 3 × 10-Fold Cross-Validation Tested
on Demilitarized Data. Average and standard deviation of rankings of
z-scores for accuracy, F1 score, and ROCAUC are presented for all models.

| | Traditional Methods | | | Δ Classifiers | |
|---|---|---|---|---|---|
| Metric | RF | XGB | CP | ΔCL | DΔC |
| Accuracy | 2.907 ± 0.799 | 3.837 ± 0.595 | 4.835 ± 0.560 | 2.130 ± 0.724 | 1.291 ± 0.652 |
| F1 Score | 2.913 ± 0.801 | 3.841 ± 0.593 | 4.830 ± 0.578 | 2.128 ± 0.716 | 1.287 ± 0.637 |
| ROCAUC | 2.857 ± 0.788 | 3.865 ± 0.587 | 4.813 ± 0.564 | 2.091 ± 0.839 | 1.374 ± 0.705 |

TABLE 16
Results for 1 × 10-Fold Cross-Validation Tested Without Same Molecule Pairs
and Bounded Data. Average and standard deviation of accuracy, F1 score, and ROCAUC
are presented for all models following removal of molecular pairs of the same molecule
and molecular pairs incorporating a molecule with a bounded IC₅₀ value in the test
set. Highest statistically significant performances across all models are underlined.
Highest statistically significant performances within each model family (traditional
models, tree-based Δ classifiers, and deep Δ classifiers) are bolded.

| | Traditional Methods | | | Tree-Based Δ Classifiers | | | Deep Δ Classifiers | | |
|---|---|---|---|---|---|---|---|---|---|
| Metric | RF | XGB | CP | ΔCLOE | ΔCLAD | ΔCL | DΔCOE | DΔCAD | DΔC |
| Accuracy | 0.791 ± | 0.784 ± | 0.747 ± | 0.784 ± | 0.779 ± | 0.779 ± | 0.792 ± | 0.790 ± | 0.791 ± |
| | 0.047 | 0.049 | 0.052 | 0.048 | 0.052 | 0.052 | 0.048 | 0.049 | 0.049 |
| F1 Score | 0.790 ± | 0.782 ± | 0.746 ± | 0.781 ± | 0.777 ± | 0.778 ± | 0.790 ± | 0.788 ± | 0.790 ± |
| | 0.048 | 0.050 | 0.053 | 0.050 | 0.054 | 0.053 | 0.050 | 0.052 | 0.051 |
| ROCAUC | 0.872 ± | 0.863 ± | 0.827 ± | 0.863 ± | 0.857 ± | 0.858 ± | 0.871 ± | 0.867 ± | 0.867 ± |
| | 0.049 | 0.052 | 0.062 | 0.052 | 0.058 | 0.057 | 0.051 | 0.053 | 0.052 |

TABLE 17
Results for 1 × 10-Fold Cross-Validation Tested on Demilitarized Non-Matching Scaffold
Pairs. Average and standard deviation of accuracy, F1 score, and ROCAUC are presented
for all models. Highest statistically significant performances across all models are
underlined. Highest statistically significant performances within each model family
(traditional models, tree-based Δ classifiers, and deep Δ classifiers) are bolded.

| | Traditional Methods | | | Tree-Based Δ Classifiers | | | Deep Δ Classifiers | | |
|---|---|---|---|---|---|---|---|---|---|
| Metric | RF | XGB | CP | ΔCLOE | ΔCLAD | ΔCL | DΔCOE | DΔCAD | DΔC |
| Accuracy | 0.797 ± | 0.787 ± | 0.751 ± | 0.787 ± | 0.826 ± | 0.827 ± | 0.800 ± | 0.838 ± | 0.839 ± |
| | 0.058 | 0.062 | 0.070 | 0.060 | 0.049 | 0.049 | 0.057 | 0.045 | 0.045 |
| F1 Score | 0.797 ± | 0.787 ± | 0.751 ± | 0.786 ± | 0.826 ± | 0.827 ± | 0.798 ± | 0.838 ± | 0.838 ± |
| | 0.058 | 0.062 | 0.070 | 0.062 | 0.049 | 0.049 | 0.059 | 0.046 | 0.045 |
| ROCAUC | 0.875 ± | 0.863 ± | 0.828 ± | 0.865 ± | 0.903 ± | 0.902 ± | 0.876 ± | 0.912 ± | 0.912 ± |
| | 0.063 | 0.069 | 0.082 | 0.065 | 0.048 | 0.047 | 0.060 | 0.042 | 0.042 |

TABLE 18
Ranking of Model Performance for 1 × 10-Fold Cross-Validation Tested on Demilitarized
Data for Non-Matching Scaffold Pairs. Average and standard deviation of rankings
of z-scores for accuracy, F1 score, and ROCAUC are presented for all models.

| | Traditional Methods | | | Δ Classifiers | |
|---|---|---|---|---|---|
| Metric | RF | XGB | CP | ΔCL | DΔC |
| Accuracy | 2.961 ± 0.820 | 3.778 ± 0.709 | 4.796 ± 0.596 | 2.089 ± 0.766 | 1.376 ± 0.766 |
| F1 Score | 2.941 ± 0.834 | 3.774 ± 0.711 | 4.8 ± 0.594 | 2.098 ± 0.785 | 1.387 ± 0.761 |
| ROCAUC | 2.863 ± 0.830 | 3.82 ± 0.702 | 4.785 ± 0.593 | 2.133 ± 0.839 | 1.400 ± 0.761 |

TABLE 19
Results for 1 × 10-Fold Cross-Validation Tested on Demilitarized Matching Scaffold
Pairs. Average and standard deviation of accuracy, F1 score, and ROCAUC are presented
for all models. Highest statistically significant performances across all models are
underlined. Highest statistically significant performances within each model family
(traditional models, tree-based Δ classifiers, and deep Δ classifiers) are bolded.

| | Traditional Methods | | | Tree-Based Δ Classifiers | | | Deep Δ Classifiers | | |
|---|---|---|---|---|---|---|---|---|---|
| Metric | RF | XGB | CP | ΔCLOE | ΔCLAD | ΔCL | DΔCOE | DΔCAD | DΔC |
| Accuracy | 0.670 ± | 0.670 ± | 0.601 ± | 0.657 ± | 0.674 ± | 0.675 ± | 0.678 ± | 0.701 ± | 0.699 ± |
| | 0.099 | 0.093 | 0.097 | 0.087 | 0.084 | 0.080 | 0.092 | 0.085 | 0.083 |
| F1 Score | 0.668 ± | 0.667 ± | 0.601 ± | 0.635 ± | 0.660 ± | 0.676 ± | 0.669 ± | 0.695 ± | 0.699 ± |
| | 0.101 | 0.095 | 0.097 | 0.107 | 0.092 | 0.079 | 0.102 | 0.088 | 0.083 |
| ROCAUC | 0.733 ± | 0.723 ± | 0.638 ± | 0.720 ± | 0.744 ± | 0.744 ± | 0.740 ± | 0.768 ± | 0.767 ± |
| | 0.114 | 0.116 | 0.124 | 0.108 | 0.100 | 0.099 | 0.114 | 0.099 | 0.098 |

TABLE 20
Ranking of Model Performance for 1 × 10-Fold Cross-Validation Tested on Demilitarized
Data for Matching Scaffold Pairs. Average and standard deviation of rankings of
z-scores for accuracy, F1 score, and ROCAUC are presented for all models.

| | Traditional Methods | | | Δ Classifiers | |
|---|---|---|---|---|---|
| Metric | RF | XGB | CP | ΔCL | DΔC |
| Accuracy | 2.748 ± 1.149 | 2.941 ± 1.222 | 4.398 ± 1.046 | 2.989 ± 1.183 | 1.924 ± 1.190 |
| F1 Score | 2.772 ± 1.145 | 2.983 ± 1.236 | 4.370 ± 1.078 | 2.952 ± 1.192 | 1.924 ± 1.208 |
| ROCAUC | 2.757 ± 1.122 | 3.100 ± 1.173 | 4.498 ± 0.981 | 2.759 ± 1.216 | 1.887 ± 1.155 |

What is claimed:

1. A computer-implemented method for training a machine learning model for predicting molecular property differences, the method comprising:
receiving a set of data including molecules, wherein each molecule of the set of data includes a molecular representation and a known absolute property value of each molecule in the set of data;
creating a set of training data with the set of data;
creating a set of molecule pairs using each molecule of the set of training data;
generating a shared molecular representation of each pair of molecules in the set of training data;
training a machine learning model of an artificial intelligence (AI) system using the set of training data, wherein the set of training data includes the shared molecular representation and property difference of each pair of molecules in the set of training data; and
for two molecules forming a molecule pair, predicting a property difference of molecular derivatization using the machine learning model as trained based on property differences of each pair of molecules.

2. The method of claim 1, wherein creating the set of molecule pairs using each molecule of the set of training data, further comprises:
splitting the set of training data into a training set and a test set.

3. The method of claim 2, wherein creating the set of molecule pairs using each molecule of the set of training data, further comprises:
generating a pair of molecules for at least one of the training set and the test set by cross merging a first molecule of the training set or the test set and a second molecule of the training set or the test set, wherein all possible molecule pairs of the training set and the test set are generated, wherein cross merging of the training set is limited to molecules of the training set, and wherein cross merging of the test set is limited to molecules of the test set.

4. The method of claim 1, wherein generating a shared molecular representation of each pair of molecules in the set of training data, further comprises:
concatenating a first molecular representation of a first molecule and a second molecular representation of a second molecule of each pair of molecules in the set of training data.

5. A computer program product comprising program instructions stored on a machine-readable storage medium, wherein when the program instructions are executed by a computer processor, the program instructions cause the computer processor to execute the method as claimed in claim 1.

6. A computer-implemented method for training a machine learning model for retrieving a compound with a desired characteristic from a set of data, the method comprising:
receiving a set of data including molecules, wherein each molecule of the set of data includes a molecular representation and a known absolute property;
creating a set of training data based on the set of data;
creating a set of molecule pairs using each molecule of the set of training data;
generating a shared molecular representation of each pair of molecules in the set of molecule pairs;
training a machine learning model of an AI system using the set of training data, wherein the set of training data includes the shared molecular representation and respective property differences of each pair of molecules of the set of training data;
identifying a first compound of the set of training data based on a property of the identified compound;
pairing the identified compound with each compound of a learning dataset, wherein the learning data set is based on the set of data;
for a pair of molecules from the learning dataset, predicting a property difference of the pair of molecules from the learning dataset using the machine learning model as trained based on property differences of each pair of molecules of the set of training data, wherein the pair of molecules include the identified compound; and
adding a compound paired with the identified compound from the learning dataset to the training data set based on a property increase of the compound and the identified compound.

7. The method of claim 6, further comprising:
creating a second set of molecule pairs using each molecule of the set of training data, wherein the set of training data includes the added compound; and
retraining the machine learning model using the set of training data, wherein the set of training data includes shared molecular representations and respective property differences of each pair of molecules of the second set of molecule pairs of the set of training data.

8. The method of claim 6, wherein the compound paired with the identified compound has a property improvement greater than other compounds paired with the identified compound in the learning dataset.

9. The method of claim 6, wherein creating the set of molecule pairs using each molecule of the set of training data, further comprises:
splitting the set of training data into one or more sets selected from the group consisting of: a training set, a test set, and the learning dataset.

10. The method of claim 9, wherein creating the set of molecule pairs using each molecule of the set of training data, further comprises:
generating a pair of molecules for at least one of the training set, the test set, and the learning dataset by cross merging a first molecule and a second molecule of the training set, the test set, or the learning dataset, wherein all possible molecule pairs of the training set, the test set, or the learning dataset are generated and the learning set is cross merged with one molecule of the training set, wherein cross merging of the training set is limited to molecules of the training set, wherein cross merging of the test set is limited to molecules of the test set, and wherein cross merging of the learning set is limited to the one molecule of the training set and molecules of the learning set, wherein the one molecule of the training set includes a desired property value.

11. The method of claim 10, further comprising:
for a pair of molecules from an external dataset, predicting a property difference of the pair of molecules from the external dataset using the machine learning model as trained based on property differences of each pair of molecules of the set of training data.

12. A computer program product comprising program instructions stored on a machine-readable storage medium, wherein when the program instructions are executed by a computer processor, the program instructions cause the computer processor to execute the method as claimed in claim 6.

13. A computer-implemented method for training a machine learning model for predicting which of a pair of molecules has an improved property value, the method comprising:
receiving a set of data including molecules, wherein each molecule of the set of data includes a molecular representation and a value selected from a group consisting of: a known exact absolute property value and a known bound absolute property value, wherein the known exact absolute property value and the known bound absolute property value are related to a property of each molecule of the set of data;
creating a set of training data with the set of data;
creating a set of molecule pairs using each molecule of the set of training data;
filtering the set of training data based on a set of rules, wherein the filtered set of training data includes molecule pairs of the set of molecule pairs having at least one molecule with a property value improved compared to the other molecule;
training a machine learning model of an AI system using datapoints of the filtered set of training data, wherein the datapoints include molecular pairs of the filtered set of training data with shared representations, and wherein the datapoints include at least one selected from the group consisting of: bounded datapoints and exact regression datapoints; and
for datapoints of a pair of molecules, predicting a property value improvement of molecular derivatization using the machine learning model as trained based on property differences of the datapoints, wherein the property value improvement indicates at least one molecule of the pair of molecules includes a property value greater than the other molecule.

14. The method of claim 13, wherein filtering the set of training data based on the set of rules, further comprises:
removing, from the set of training data, molecular pairs of the set of training data with a property difference below a property difference threshold value.

15. The method of claim 13, wherein filtering the set of training data based on the set of rules, further comprises:
removing, from the set of training data, molecular pairs of the set of training data having a first molecule and a second molecule with equal property values.

16. The method of claim 13, wherein filtering the set of training data based on the set of rules, further comprises:
removing, from the set of training data, molecular pairs of the set of training data when an improved property is unknown.

17. The method of claim 13, wherein creating the set of molecule pairs using each molecule of the set of training data, further comprises:
splitting the set of training data into a training set and a test set.

18. The method of claim 17, wherein creating the set of molecule pairs using each molecule of the set of training data, further comprises:
generating a pair of molecules for at least one of the training set and the test set by cross merging a first molecule and a second molecule of the training set and the test set, wherein all possible molecule pairs of the training set and the test set are generated, wherein cross merging of the training set is limited to molecules of the training set, and wherein cross merging of the test set is limited to molecules of the test set.

19. A computer program product comprising program instructions stored on a machine-readable storage medium, wherein when the program instructions are executed by a computer processor, the program instructions cause the computer processor to execute the method as claimed in claim 13.
